(19) United States
(12) Patent Application Publication
(10) Pub. No.: US 2024/0167993 A1
(43) Pub. Date: May 23, 2024

(54) PREDICTION OF INDOOR BIOAEROSOL CONCENTRATIONS FROM INDOOR AIR QUALITY SENSOR DATA BY ARTIFICIAL INTELLIGENCE MODELS

(71) Applicant: City University of Hong Kong, Kowloon (HK)

(72) Inventors: Yik Yeung Lee, Kowloon (HK); Lik Tak Ricky Chau, Lam Tin (HK); Yanhao Miao, Wong Tai Sin District (HK); Patrick Kwan Hon Lee, Shatin (HK)

(21) Appl. No.: 17/992,232

(22) Filed: Nov. 22, 2022

(51) Int. Cl. G01N 33/00

(52) U.S. Cl. CPC G01N 33/0075; G01N 33/0016; G01N 33/0034

(57) ABSTRACT

A method for predicting concentration of indoor bioaerosols. The method contains the steps of providing a plurality of AI models, evaluating a prediction accuracy of each of the plurality of AI models for a venue; choosing a best model from the plurality of AI models for the venue; inputting measured data at the venue into the best model; and generating a prediction of concentration of indoor bioaerosols by the best model for the venue. To accurately monitor and predict the indoor concentration of bioaerosols, a novel methodology for predicting real-time and near-future concentration of indoor bioaerosols with artificial intelligence (AI) models is thus presented.

### FIELD OF INVENTION

This invention relates to air quality monitoring, and in particular to the monitoring and prediction of indoor bioaerosol concentrations.

### BACKGROUND OF INVENTION

People spend more than 85% of their time indoors,¹⁻³ which means that indoor air quality (IAQ) significantly affects human health.³ As such, poor IAQ causes building-associated illness.⁴ Given that ˜5%-34% of particulate matter (PM) in indoor air is in the form of bioaerosols (i.e., bacteria, fungi and pollen),⁵ these particles are gaining increasing research attention,⁵⁻⁷ especially as the coronavirus disease 2019 (COVID-19) pandemic continues.^{8,9}

Culturing-based methods have traditionally been used to determine the concentration of bioaerosols,^{10,11} but as these require offline processing and a long incubation time they cannot supply real-time information. Furthermore, because many microorganisms are known to be unculturable under standard laboratory conditions,¹² bioaerosol concentrations are typically underestimated.¹³ Alternatively, ultraviolet light/laser-induced fluorescence techniques can be used to determine the concentrations and identities of bioaerosols in real time.¹⁴⁻¹⁸ However, the instruments required for these analyses are large and expensive, which makes them impractical for widespread deployment in indoor environments.

Artificial intelligence (AI)-based methods, such as machine learning and deep learning models, have been developed for the prediction of IAQ and applied to predict the trends in the values of IAQ parameters using data measured by real-time sensors.¹⁹ An artificial neural network, a form of deep learning model, was used to accurately determine the future concentration of carbon dioxide (CO₂) in an office from past data.²⁰ Similarly, deep learning models based on long short-term memory (LSTM) and gated recurrent units (GRUs) were developed to forecast trends in the concentrations of CO₂ and fine dust in an office based on the past data of six IAQ parameters.²¹ In another example, size-segregated particle concentrations, temperature and relative humidity (RH) were fitted to a multi-linear regression model, enabling it to predict the concentrations of airborne bacteria and fungi in a hospital from culture-based data.²²

However, no method has been developed that can accurately determine real-time and near future bioaerosol concentrations on a continuous basis.

### REFERENCES

Each of the following references (and associated appendices and/or supplements) is expressly incorporated herein by reference in its entirety:
(1) Leech, J. A.; Nelson, W. C.; Burnett, R. T.; Aaron, S.; Raizenne, M. E. It's about time: A comparison of Canadian and American time-activity patterns. J. Expo. Sci. Environ. Epidemiol. 2002, 12 (6), 427-432. https://doi.org/10.1038/sj.jea.7500244.
(2) Klepeis, N. E.; Nelson, W. C.; Ott, W. R.; Robinson, J. P.; Tsang, A. M.; Switzer, P.; Behar, J. V.; Hem, S. C.; Engelmann, W. H. The National Human Activity Pattern Survey (NHAPS): A resource for assessing exposure to environmental pollutants. J. Expo. Sci. Environ. Epidemiol. 2001, 11 (3), 231-252. https://doi.org/10.1038/sj.jea.7500165.
(3) Cincinelli, A.; Martellini, T. Indoor air quality and health. Int. J. Environ. Res. Public. Health 2017, 14 (11), 1286. https://doi.org/10.3390/ijerph14111286.
(4) Hromadka, J.; Korposh, S.; Partridge, M. C.; James, S. W.; Davis, F.; Crump, D.; Tatam, R. P. Multi-parameter measurements using optical fibre long period gratings for indoor air quality monitoring. Sens. Actuators B Chem. 2017, 244, 217-225. https://doi.org/10.1016/j.snb.2016.12.050.
(5) Kim, K.-H.; Kabir, E.; Jahan, S. A. Airborne bioaerosols and their impact on human health. J. Environ. Sci. 2018, 67, 23-35. https://doi.org/10.1016/j.jes.2017.08.027.
(6) Marcovecchio, F.; Perrino, C. Bioaerosol contribution to atmospheric particulate matter in indoor university environments. Sustainability 2021, 13 (3), 1149. https://doi.org/10.3390/su13031149.
(7) Yamamoto, N.; Hospodsky, D.; Dannemiller, K. C.; Nazaroff, W. W.; Peccia, J. Indoor emissions as a primary source of airborne allergenic fungal particles in classrooms. Environ. Sci. Technol. 2015, 49 (8), 5098-5106. https://doi.org/10.1021/es506165z.
(8) Tiwari, A.; Gupta, R.; Chandra, R. Delhi air quality prediction using LSTM deep learning models with a focus on COVID-19 Lockdown. arXiv Feb. 21, 2021.
(9) Agarwal, N.; Meena, C. S.; Raj, B. P.; Saini, L.; Kumar, A.; Gopalakrishnan, N.; Kumar, A.; Balam, N. B.; Alam, T.; Kapoor, N. R.; Aggarwal, V. Indoor air quality improvement in COVID-19 pandemic: Review. Sustain. Cities Soc. 2021, 70, 102942. https://doi.org/10.1016/j.scs.2021.102942.
(10) Schäfer, J.; Weiß, S.; Jäckel, U. Preliminary validation of a method combining cultivation and cloning-based approaches to monitor airborne bacteria. Ann. Work Expo. Health 2017, 61 (6), 633-642. https://doi.org/10.1093/annweh/wxx038.
(11) Duquenne, P. On the identification of culturable microorganisms for the assessment of biodiversity in bioaerosols. Ann. Work Expo. Health 2018, 62 (2), 139-146. https://doi.org/10.1093/annweh/wxxO96.
(12) Lloyd, K. G.; Steen, A. D.; Ladau, J.; Yin, J.; Crosby, L. Phylogenetically novel uncultured microbial cells dominate Earth microbiomes. mSystems 2018, 3 (5), e00055-18. https://doi.org/10.1128/mSystems.00055-18.
(13) Chi, M.-C.; Li, C.-S. Analysis of bioaerosols from chicken houses by culture and non-culture method. Aerosol Sci. Technol. 2006, 40 (12), 1071-1079. https://doi.org/10.1080/02786820600957408.
(14) Huffman, J. A.; Perring, A. E.; Savage, N. J.; Clot, B.; Crouzy, B.; Tummon, F.; Shoshanim, O.; Damit, B.; Schneider, J.; Sivaprakasam, V.; Zawadowicz, M. A.; Crawford, I.; Gallagher, M.; Topping, D.; Doughty, D. C.; Hill, S. C.; Pan, Y. Real-time sensing of bioaerosols: Review and current perspectives. Aerosol Sci. Technol. 2020, 54 (5), 465-495. https://doi.org/10.1080/02786826.2019.1664724.
(15) Hernandez, M.; Perring, A. E.; McCabe, K.; Kok, G.; Granger, G.; Baumgardner, D. Chamber catalogues of optical and fluorescent signatures distinguish bioaerosol classes. Atmospheric Meas. Tech. 2016, 9 (7), 3283-3292. https://doi.org/10.5194/amt-9-3283-2016.
(16) Nieto-Caballero, M.; Gomez, O. M.; Shaughnessy, R.; Hernandez, M. Aerosol fluorescence, airborne hexosaminidase, and quantitative genomics distinguish reductions in airborne fungal loads following major school renovations. Indoor Air 2022, 32 (1). https://doi.org/10.1111/ina.12975.
(17) Tian, Y.; Liu, Y.; Misztal, P. K.; Xiong, J.; Arata, C. M.; Goldstein, A. H.; Nazaroff, W. W. Fluorescent biological aerosol particles: Concentrations, emissions, and exposures in a Northern California residence. Indoor Air 2018, 28 (4), 559-571. https://doi.org/10.1111/ina.12461.
(18) Li, J.; Zuraimi, S.; Schiavon, S.; Wan, M. P.; Xiong, J.; Tham, K. W. Diurnal trends of indoor and outdoor fluorescent biological aerosol particles in a tropical urban area. Sci. Total Environ. 2022, 848, 157811. https://doi.org/10.1016/j.scitotenv.2022.157811.
(19) Wei, W.; Ramalho, O.; Malingre, L.; Sivanantham, S.; Little, J. C.; Mandin, C. Machine learning and statistical models for predicting indoor air quality. Indoor Air 2019, 29 (5), 704-726. https://doi.org/10.1111/ina.12580.
(20) Putra, J. C. P.; Safrilah; Ihsan, M. The prediction of indoor air quality in office room using artificial neural network. In AIP Conference Proceedings; Surakarta, Indonesia, 2018; Vol. 1977, p 020040. https://doi.org/10.1063/1.5042896.
(21) Ahn, J.; Shin, D.; Kim, K.; Yang, J. Indoor air quality analysis using deep learning with sensor data. Sensors 2017, 17 (11), 2476. https://doi.org/10.3390/s17112476.
(22) Seo, J. H.; Jeon, H. W.; Choi, J. S.; Sohn, J.-R. Prediction model for airborne microorganisms using particle number concentration as surrogate markers in hospital environment. Int. J. Environ. Res. Public. Health 2020, 17 (19), 7237. https://doi.org/10.3390/ijerph17197237.
(23) Rong, S.; Bao-wen, Z. The research of regression model in machine learning field. In 6th International Forum on Industrial Design (IFID 2018); 2018; Vol. 176, p 01033. https://doi.org/10.1051/matecconf/201817601033.
(24) Kwon, S.; Han, S.; Lee, S. A small review and further studies on the LASSO. J Korean Data Inf Sci. Soc. 2013, 24 (5), 1077-1088. https://doi.org/10.7465/jkdi.2013.24.5.1077.
(25) Tibshirani, R. Regression shrinkage and selection via the lasso. J. R. Stat. Soc. Ser. B Methodol. 1996, 58 (1), 267-288. https://doi.org/10.1111/j.2517-6161.1996.tb02080.x.
(26) Breiman, L. Random forests. Mach. Learn. 2001, 45 (1), 5-32. https://doi.org/10.1023/A:1010933404324.
(27) Chen, T.; Guestrin, C. XGBoost: A scalable tree boosting system. In Proceedings of the 22nd ACM SIGKDD International Conference on Knowledge Discovery and Data Mining; ACM: San Francisco California USA, 2016; pp 785-794. https://doi.org/10.1145/2939672.2939785.
(28) Jain, A. K.; Jianchang Mao; Mohiuddin, K. M. Artificial neural networks: A tutorial. Computer 1996, 29 (3), 31-44. https://doi.org/10.1109/2.485891.
(29) Hochreiter, S.; Schmidhuber, J. Long short-term memory. Neural Comput. 1997, 9 (8), 1735-1780. https://doi.org/10.1162/neco.1997.9.8.1735.
(30) Sherstinsky, A. Fundamentals of recurrent neural network (RNN) and long short-term memory (LSTM) network. Phys. Nonlinear Phenom. 2020, 404, 132306. https://doi.org/10.1016/j.physd.2019.132306.
(31) Willmott, C. J.; Robeson, S. M.; Matsuura, K. A refined index of model performance. Int. J. Climatol. 2012, 32 (13), 2088-2094. https://doi.org/10.1002/joc.2419.
(32) Pedregosa, F.; Varoquaux, G.; Gramfort, A.; Michel, V.; Thirion, B.; Grisel, O.; Blondel, M.; Prettenhofer, P.; Weiss, R.; Dubourg, V.; Vanderplas, J.; Passos, A.; Cournapeau, D.; Brucher, M.; Perrot, M.; Duchesnay, É. Scikit-learn: Machine learning in Python. J. Mach. Learn. Res. 2011, 12 (85), 2825-2830.
(33) Virtanen, P.; Gommers, R.; Oliphant, T. E.; Haberland, M.; Reddy, T.; Cournapeau, D.; Burovski, E.; Peterson, P.; Weckesser, W.; Bright, J.; van der Walt, S. J.; Brett, M.; Wilson, J.; Millman, K. J.; Mayorov, N.; Nelson, A. R. J.; Jones, E.; Kern, R.; Larson, E.; Carey, C. J.; Polat, I.; Feng, Y.; Moore, E. W.; VanderPlas, J.; Laxalde, D.; Perktold, J.; Cimrman, R.; Henriksen, I.; Quintero, E. A.; Harris, C. R.; Archibald, A. M.; Ribeiro, A. H.; Pedregosa, F.; van Mulbregt, P.; SciPy 1.0 Contributors; Vijaykumar, A.; Bardelli, A. P.; Rothberg, A.; Hilboll, A.; Kloeckner, A.; Scopatz, A.; Lee, A.; Rokem, A.; Woods, C. N.; Fulton, C.; Masson, C.; Haggström, C.; Fitzgerald, C.; Nicholson, D. A.; Hagen, D. R.; Pasechnik, D. V.; Olivetti, E.; Martin, E.; Wieser, E.; Silva, F.; Lenders, F.; Wilhelm, F.; Young, G.; Price, G. A.; Ingold, G.-L.; Allen, G. E.; Lee, G. R.; Audren, H.; Probst, I.; Dietrich, J. P.; Silterra, J.; Webber, J. T.; Slavic, J.; Nothman, J.; Buchner, J.; Kulick, J.; Schönberger, J. L.; de Miranda Cardoso, J. V.; Reimer, J.; Harrington, J.; Rodriguez, J. L. C.; Nunez-Iglesias, J.; Kuczynski, J.; Tritz, K.; Thoma, M.; Newville, M.; Kummerer, M.; Bolingbroke, M.; Tartre, M.; Pak, M.; Smith, N. J.; Nowaczyk, N.; Shebanov, N.; Pavlyk, O.; Brodtkorb, P. A.; Lee, P.; McGibbon, R. T.; Feldbauer, R.; Lewis, S.; Tygier, S.; Sievert, S.; Vigna, S.; Peterson, S.; More, S.; Pudlik, T.; Oshima, T.; Pingel, T. J.; Robitaille, T. P.; Spura, T.; Jones, T. R.; Cera, T.; Leslie, T.; Zito, T.; Krauss, T.; Upadhyay, U.; Halchenko, Y. O.; Vázquez-Baeza, Y. SciPy 1.0: Fundamental algorithms for scientific computing in Python. Nat. Methods 2020, 17 (3), 261-272. https://doi.org/10.1038/s41592-019-0686-2.
(34) Seabold, S.; Perktold, J. Statsmodels: Econometric and statistical modeling with Python. In 9th Python in Science Conference; 2010.
(35) Altmann, A.; Tologi, L.; Sander, O.; Lengauer, T. Permutation importance: A corrected feature importance measure. Bioinformatics 2010, 26 (10), 1340-1347. https://doi.org/10.1093/bioinformatics/btq134.
(36) Makariou, D.; Barrieu, P.; Chen, Y. A Random forest based approach for predicting spreads in the primary catastrophe bond market. Insur. Math. Econ. 2021, 101, 140-162. https://doi.org/10.1016/j.insmatheco.2021.07.003.
(37) Lagesse, B.; Wang, S.; Larson, T. V.; Kim, A. A. Predicting PM_{2.5} in well-mixed indoor air for a large office building using regression and artificial neural network models. Environ. Sci. Technol. 2020, 54 (23), 15320-15328. https://doi.org/10.1021/acs.est.0c02549.
(38) Saini, J.; Dutta, M.; Marques, G. Indoor air quality monitoring with IoT: Predicting PM₁₀ for enhanced decision support. In 2020 International Conference on Decision Aid Sciences and Application (DASA); IEEE: Sakheer, Bahrain, 2020; pp 504-508. https://doi.org/10.1109/DASA51403.2020.9317054.
(39) Li, X.; Cheng, X.; Wu, W.; Wang, Q.; Tong, Z.; Zhang, X.; Deng, D.; Li, Y. Forecasting of bioaerosol concentration by a back propagation neural network model. Sci. Total Environ. 2020, 698, 134315. https://doi.org/10.1016/j.scitotenv.2019.134315.
(40) Garrett, M. H.; Hooper, B. M.; Cole, F. M.; Hooper, M. A. Airborne fungal spores in 80 homes in the Latrobe Valley, Australia: Levels, seasonality and indoor-outdoor relationship. Aerobiologia 1997, 13 (2), 121-126. https://doi.org/10.1007/BF02694428.
(41) Leung, D. Y. C. Outdoor-indoor air pollution in urban environment: Challenges and opportunity. Front. Environ. Sci. 2015, 2. https://doi.org/10.3389/fenvs.2014.00069.
(42) Meadow, J. F.; Altrichter, A. E.; Kembel, S. W.; Kline, J.; Mhuireach, G.; Moriyama, M.; Northcutt, D.; O'Connor, T. K.; Womack, A. M.; Brown, G. Z.; Green, J. L..; Bohannan, B. J. M. Indoor airborne bacterial communities are influenced by ventilation, occupancy, and outdoor air source. Indoor Air 2014, 24 (1), 41-48. https://doi.org/10.1111/ina.12047.
(43) Hospodsky, D.; Qian, J.; Nazaroff, W. W.; Yamamoto, N.; Bibby, K.; Rismani-Yazdi, H.; Peccia, J. Human occupancy as a source of indoor airborne bacteria. PLoS ONE 2012, 7 (4), e34867. https://doi.org/10.1371/journal.pone.0034867.
(44) Xu, C.; Wu, C.-Y.; Yao, M. Fluorescent bioaerosol particles resulting from human occupancy with and without respirators. Aerosol Air Qual. Res. 2017, 17 (1), 198-208. https://doi.org/10.4209/aagr.2016.09.0400.
(45) Chen, Q.; Hildemann, L. M. The effects of human activities on exposure to particulate matter and bioaerosols in residential homes. Environ. Sci. Technol. 2009, 43 (13), 4641-4646. https://doi.org/10.1021/es802296j.
(46) Kim, H.; Kang, K.; Kim, T. Effect of occupant activity on indoor particle concentrations in Korean residential buildings. Sustainability 2020, 12 (21), 9201. https://doi.org/10.3390/su12219201.
(47) Li, W.-M.; Lee, S. C.; Chan, L. Y. Indoor air quality at nine shopping malls in Hong Kong. Sci. Total Environ. 2001, 273 (1-3), 27-40. https://doi.org/10.1016/S0048-9697(00)00833-0.
(48) Law, A. K. Y.; Chau, C. K.; Chan, G. Y. S. Characteristics of bioaerosol profile in office buildings in Hong Kong. Build. Environ. 2001, 36 (4), 527-541. https://doi.org/10.1016/S0360-1323(00)00020-2.
(49) Tsokov, S.; Lazarova, M.; Aleksieva-Petrova, A. A hybrid spatiotemporal deep model based on CNN and LSTM for air pollution prediction. Sustainability 2022, 14 (9), 5104. https://doi.org/10.3390/su14095104.
(50) Dai, H.; Huang, G.; Wang, J.; Zeng, H.; Zhou, F. Prediction of air pollutant concentration based on one-dimensional multi-scale CNN-LSTM considering spatial-temporal characteristics: A case study of Xi'an, China. Atmosphere 2021, 12 (12), 1626. https://doi.org/10.3390/atmos12121626.
(51) Pan, M.; Lednicky, J. A.; Wu, C.-Y. Collection, particle sizing and detection of airborne viruses. J. Appl. Microbiol. 2019, 127 (6), 1596-1611. https://doi.org/10.1111/jam.14278.
(52) Ribeiro, B. V.; Cordeiro, T. A. R.; Oliveira e Freitas, G. R.; Ferreira, L. F.; Franco, D. L. Biosensors for the detection of respiratory viruses: A review. Talanta Open 2020, 2, 100007. https://doi.org/10.1016/j.talo.2020.100007.
(53) Hipp, Richard D. SQLite, 2020.
(54) Chollet, Frangois; and others. Keras, 2015.
(55) TensorFlow Developers. TensorFlow, 2022. https://doi.org/10.5281/ZENODO.4724125.
(56) Kingma, D. P.; Ba, J. Adam: A method for stochastic optimization. ArXiv14126980 Cs 2017.
(57) Liashchynskyi, P.; Liashchynskyi, P. Grid search, random search, genetic algorithm: A big comparison for NAS. ArXiv191206059 Cs Stat 2019.

### SUMMARY OF INVENTION

Accordingly, the present invention, in one aspect, is a method for predicting concentration of indoor bioaerosols. The method contains the steps of providing a plurality of AI models, evaluating a prediction accuracy of each of the plurality of AI models for a venue; choosing a best model from the plurality of AI models for the venue; inputting measured data at the venue into the best model; and generating a prediction of concentration of indoor bioaerosols by the best model for the venue.

In some embodiments, the plurality of AI models includes one or more of a linear regression model, a lasso regression model, a random forest (RF) model, an extreme gradient boosting model, a multilayer perceptron model, an LSTM model, and a recurrent neural network model.

In some embodiments, the step of evaluating a prediction accuracy of each of the plurality of AI models for a venue, further includes the steps of inputting test data for the venue into each of the plurality of AI models; applying more than one pair of input and output time windows; finding, for each of the plurality of AI model, a difference data between predicted test data and measured test data; and determining one of the plurality of AI models that has a best difference data as the best model.

In some embodiments, the difference data contains one or more of a mean squared error (MSE), a root-mean-square error (RMSE) and a value on a revised version of the Willmott's index (WI).

In some embodiments, the more than one pair of input and output time windows includes a real-time window pair.

In some embodiments, the measured data contains a plurality of input features. The method further contains a step of determining which one of the plurality of input features is more important than another one by conducting a permutation importance analysis.

In some embodiments, the plurality of input features contains one or more of temperature, RH, concentrations of CO₂, total volatile organic compounds (TVOCs), PM_{2.5} and PM₁₀.

In some embodiments, the plurality of input features contains concentrations of more than one biological matters.

According to another aspect of the invention, there is provided an apparatus for predicting concentration of indoor bioaerosols. The apparatus include one or more processors; a memory storing computer-executable instructions that, when executed, cause the one or more processors to provide a plurality of AI models; evaluate a prediction accuracy of each of the plurality of AI models for a venue; choose a best model from the plurality of AI models for the venue; input measured data at the venue into the best model; and generate a prediction of concentration of indoor bioaerosols by the best model for the venue.

According to yet another aspect of the invention, there is provided a non-transitory computer readable medium, which contains executable instructions that, when executed by at least one processor, direct the at least one processor to perform a method. The method includes providing a plurality of AI models; evaluating a prediction accuracy of each of the plurality of AI models for a venue; choosing a best model from the plurality of AI models for the venue; inputting measured data at the venue into the best model; and generating a prediction of concentration of indoor bioaerosols by the best model for the venue.

One can see that exemplary embodiments of the invention provide a method for predicting real-time and near-future concentration of indoor bioaerosols with AI models, which enables accurately monitoring and predicting the indoor concentration of bioaerosols. The method may generate a suitable AI model for predicting the concentration of bioaerosols in various indoor venues by training the model with the IAQ data collected in those venues. The AI model can render predictions of the concentrations of indoor bioaerosols (such as bacteria, fungi, and pollen) by only using specific IAQ sensor data (such as temperature, relative humidity, carbon dioxide, total volatile organic compounds, PM_{2.5} and PM₁₀) as input features. Before training the AI models, the training dataset with the input features is firstly prepared from a data-processing step and then fed into multiple different AI models, which can produce the real-time or near-future indoor concentrations of bioaerosols as outputs. By a specific set of evaluation metrics, the most suitable AI model will be chosen for each testing location. Also, by specifying different time lengths of historical input features, the AI model can forecast the indoor concentrations of bioaerosols (e.g. up to 60 minutes) in the future. The method provides a viable solution to industry and the general public to get information on the indoor bioaerosols with commonly available IAQ sensors, and make a better indoor environment to protect human health.

The foregoing summary is neither intended to define the invention of the application, which is measured by the claims, nor is it intended to be limiting as to the scope of the invention in any way.

### BRIEF DESCRIPTION OF FIGURES

The foregoing and further features of the present invention will be apparent from the following description of embodiments which are provided by way of example only in connection with the accompanying figures, of which:

FIG. 1 is the illustration of a system for monitoring and forecasting indoor bioaerosol concentration, according to a first embodiment of the invention.

FIG. 2 shows main steps of a method for making indoor bioaerosol concentration forecasting, including using a determined best AI model for monitoring and forecasting operations by a system similar to that in FIG. 1.

FIG. 3 shows the workflow of development of AI models for predicting concentrations of bioaerosols and PM in indoor air, as part of the method in FIG. 2.

FIG. 4a is a table showing optimized hyperparameters of seven AI models in five time windows for a commercial office.

FIG. 4b is a table showing optimized hyperparameters of the seven AI models in the five time windows for a shopping mall.

FIG. 5a shows boxplots of nine measured IAQ parameters for the commercial office during weekdays.

FIG. 5b shows boxplots of the nine measured IAQ parameters for the commercial office during office hours.

FIG. 6a shows boxplots of nine measured IAQ parameters for the shopping mall during weekdays and weekends.

FIG. 6b shows boxplots of the nine measured IAQ parameters for the shopping mall during store hours on weekdays.

FIG. 6c shows boxplots of the nine measured IAQ parameters for the shopping mall during store hours on weekends.

FIG. 7 shows Pairwise Pearson's correlations between nine measured IAQ parameters of the commercial office during office hours.

FIG. 8a shows Pairwise Pearson's correlations between the nine measured IAQ parameters of the shopping mall during store hours for both weekdays and weekends combined.

FIG. 8b shows Pairwise Pearson's correlations between the nine measured IAQ parameters of the shopping mall during store hours for weekdays only.

FIG. 8c shows Pairwise Pearson's correlations between the nine measured IAQ parameters of the shopping mall during store hours for weekends only.

FIG. 9a is a table showing revised WI values of the predictions made by the seven AI models in five time windows for the target features in the commercial office.

FIG. 9b is a table showing revised MSE values of the predictions made by the seven AI models in the five time windows for the target features in the commercial office.

FIG. 9c is a table showing revised RMSE values of the predictions made by the seven AI models in the five time windows for the target features in the commercial office.

FIG. 10a is a table showing revised WI values of the predictions made by the seven AI models in the five time windows for the target features in the shopping mall.

FIG. 10b is a table showing revised MSE values of the predictions made by the seven AI models in the five time windows for the target features in the shopping mall.

FIG. 10c is a table showing revised RMSE values of the predictions made by the seven AI models in the five time windows for the target features in the shopping mall.

FIG. 11a1 shows linear regression (in solid line) of the measured and LSTM model-predicted values for two target features for the commercial office, for the time window of real-time prediction.

FIG. 11a2 shows linear regression (in solid line) of the measured and LSTM model-predicted values for another two target features for the commercial office, for the time window of real-time prediction.

FIG. 11a3 shows, for the commercial office, linear regression (in solid line) of the measured and LSTM model-predicted values for another target feature for the time window of real-time prediction, and linear regression (in solid line) of the measured and LSTM model-predicted values for a target feature for the time window of 60-60.

FIG. 11a4 shows linear regression (in solid line) of the measured and LSTM model-predicted values for another two target features for the commercial office, for the time window of 60-60.

FIG. 11a5 shows linear regression (in solid line) of the measured and LSTM model-predicted values for another two target features for the commercial office, for the time window of 60-60.

FIG. 11b1 shows linear regression (in solid line) of the measured and LSTM model-predicted values for two target features for the shopping mall, for the time window of real-time prediction.

FIG. 11b2 shows linear regression (in solid line) of the measured and LSTM model-predicted values for another two target features for the shopping mall, for the time window of real-time prediction.

FIG. 11b3 shows, for the shopping mall, linear regression (in solid line) of the measured and LSTM model-predicted values for another target feature for the time window of real-time prediction, and linear regression (in solid line) of the measured and LSTM model-predicted values for a target feature for the time window of 60-60.

FIG. 11b4 shows linear regression (in solid line) of the measured and LSTM model-predicted values for another two target features for the shopping mall, for the time window of 60-60.

FIG. 11b5 shows linear regression (in solid line) of the measured and LSTM model-predicted values for another two target features for the shopping mall, for the time window of 60-60.

FIG. 11c1 shows linear regression (in solid line) of the measured and the LSTM model-predicted values for two target features for the commercial office for the time windows of 10-5.

FIG. 11c2 shows linear regression (in solid line) of the measured and the LSTM model-predicted values for another two target features for the commercial office for the time windows of 10-5.

FIG. 11c3 shows, for the commercial office, linear regression (in solid line) of the measured and the LSTM model-predicted values for another target feature for the time windows of 10-5, and linear regression (in solid line) of the measured and the LSTM model-predicted values for a target feature for the time windows of 30-15.

FIG. 11c4 shows linear regression (in solid line) of the measured and the LSTM model-predicted values for another two target features for the commercial office for the time windows of 30-15.

FIG. 11c5 shows linear regression (in solid line) of the measured and the LSTM model-predicted values for another two target features for the commercial office for the time windows of 30-15.

FIG. 11c6 shows linear regression (in solid line) of the measured and the LSTM model-predicted values for two target features for the commercial office for the time windows of 60-30.

FIG. 11c7 shows linear regression (in solid line) of the measured and the LSTM model-predicted values for another two target features for the commercial office for the time windows of 60-30.

FIG. 11c8 shows linear regression (in solid line) of the measured and the LSTM model-predicted values for another target feature for the commercial office for the time windows of 60-30.

FIG. 11d1 shows linear regression (in solid line) of the measured and the LSTM model-predicted values for two target features for the shopping mall for the time window of 10-5.

FIG. 11d2 shows linear regression (in solid line) of the measured and the LSTM model-predicted values for another two target features for the shopping mall for the time windows of 10-5.

FIG. 11d3 shows, for the shopping mall, linear regression (in solid line) of the measured and the LSTM model-predicted values for another target feature for the time windows of 10-5, and linear regression (in solid line) of the measured and the LSTM model-predicted values for a target feature for the time windows of 30-15.

FIG. 11d4 shows linear regression (in solid line) of the measured and the LSTM model-predicted values for another two target features for the shopping mall for the time windows of 30-15.

FIG. 11d5 shows linear regression (in solid line) of the measured and the LSTM model-predicted values for another two target features for the shopping mall for the time windows of 30-15.

FIG. 11d6 shows linear regression (in solid line) of the measured and the LSTM model-predicted values for two target features for the shopping mall for the time windows of 60-30.

FIG. 11d7 shows linear regression (in solid line) of the measured and the LSTM model-predicted values for another two target features for the shopping mall for the time windows of 60-30.

FIG. 11d8 shows linear regression (in solid line) of the measured and the LSTM model-predicted values for another target feature for the shopping mall for the time windows of 60-30.

FIG. 12a1 illustrates plots of the measured and LSTM model-predicted values for three target features for the commercial office time-series dataset for the time window of real-time prediction.

FIG. 12a2 illustrates plots of the measured and LSTM model-predicted values for another two target features for the commercial office time-series dataset for the time window of real-time prediction.

FIG. 12b1 illustrates plots of the measured and LSTM model-predicted values for three target features for the commercial office time-series dataset for the 10-5 time windows.

FIG. 12b2 illustrates plots of the measured and LSTM model-predicted values for another two target features for the commercial office time-series dataset for the 10-5 time windows.

FIG. 12c1 illustrates plots of the measured and LSTM model-predicted values for three target features for the commercial office time-series dataset for the 30-15 time windows.

FIG. 12c2 illustrates plots of the measured and LSTM model-predicted values for another two target features for the commercial office time-series dataset for the 30-15 time windows.

FIG. 12d1 illustrates plots of the measured and LSTM model-predicted values for three target features for the commercial office time-series dataset for the 60-30 time windows.

FIG. 12d2 illustrates plots of the measured and LSTM model-predicted values for another two target features for the commercial office time-series dataset for the 60-30 time windows.

FIG. 12e1 illustrates plots of the measured and LSTM model-predicted values for three target features for the commercial office time-series dataset for the 60-60 time windows.

FIG. 12e2 illustrates plots of the measured and LSTM model-predicted values for another two target features for the commercial office time-series dataset for the 60-60 time windows.

FIG. 13a1 illustrates plots of the measured and LSTM model-predicted values for three target features for the shopping mall time-series dataset for the time windows of real-time prediction.

FIG. 13a2 illustrates plots of the measured and LSTM model-predicted values for another two target features for the shopping mall time-series dataset for the time windows of real-time prediction.

FIG. 13b1 illustrates plots of the measured and LSTM model-predicted values for three target features for the shopping mall time-series dataset for the 10-5 time windows.

FIG. 13b2 illustrates plots of the measured and LSTM model-predicted values for another two target features for the shopping mall time-series dataset for the 10-5 time windows.

FIG. 13c1 illustrates plots of the measured and LSTM model-predicted values for three target features for the shopping mall time-series dataset for the 30-15 time windows.

FIG. 13c2 illustrates plots of the measured and LSTM model-predicted values for another two target features for the shopping mall time-series dataset for the 30-15 time windows.

FIG. 13d1 illustrates plots of the measured and LSTM model-predicted values for three target features for the shopping mall time-series dataset for the 60-30 time windows.

FIG. 13d2 illustrates plots of the measured and LSTM model-predicted values for another two target features for the shopping mall time-series dataset for the 60-30 time windows.

FIG. 13e1 illustrates plots of the measured and LSTM model-predicted values for three target features for the shopping mall time-series dataset for the 60-60 time windows.

FIG. 13e2 illustrates plots of the measured and LSTM model-predicted values for another two target features for the shopping mall time-series dataset for the 60-60 time windows.

### DETAILED DESCRIPTION

As will be described in details below, in a first embodiment of the invention machine learning and deep learning models are developed which can accurately predict continuous real-time concentrations of bioaerosols using input from a typical IAQ sensor that measures the physical and chemical properties of indoor air. The models are then trained and their performance tested using data that are obtained by measuring the physical, chemical and biological characteristics of the indoor air in an operating commercial office and a shopping mall. In addition, hyperparameters of the models are optimized and it is explored how using various time windows of past data as inputs affect the output data. In one exemplary configuration, the best model determined the per-minute concentration of bioaerosols up to 60 min into the future with ˜60%-80% accuracy. This constitutes a practical and economical strategy for assessing indoor concentrations of bioaerosols to facilitate the protection of human health.

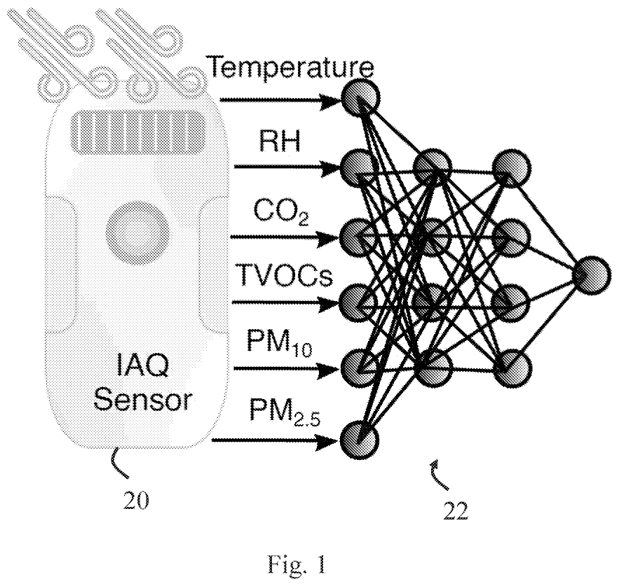

FIG. 1 shows the general configuration of an indoor bioaerosols concentration monitoring and forecasting system of the first embodiment. The system contains a real-time IAQ sensor 20 which is adapted to measure physical and chemical properties of indoor air. The IAQ sensor 20 could be any IAQ sensor available in the market, such as those of commercial grade. A typical commercial grade IAQ sensor is adapted to collect six IAQ parameters, including temperature, RH, CO₂, TVOCs, PM_{2.5}, and PM₁₀. The IAQ sensor 20 is connected to a computing device (not shown) on which an AI model 22 is carried and executed. The IAQ parameters may be provided to the AI model 22 by the IAQ sensor 20 as input features in indoor environments to predict indoor bioaerosols concentration.

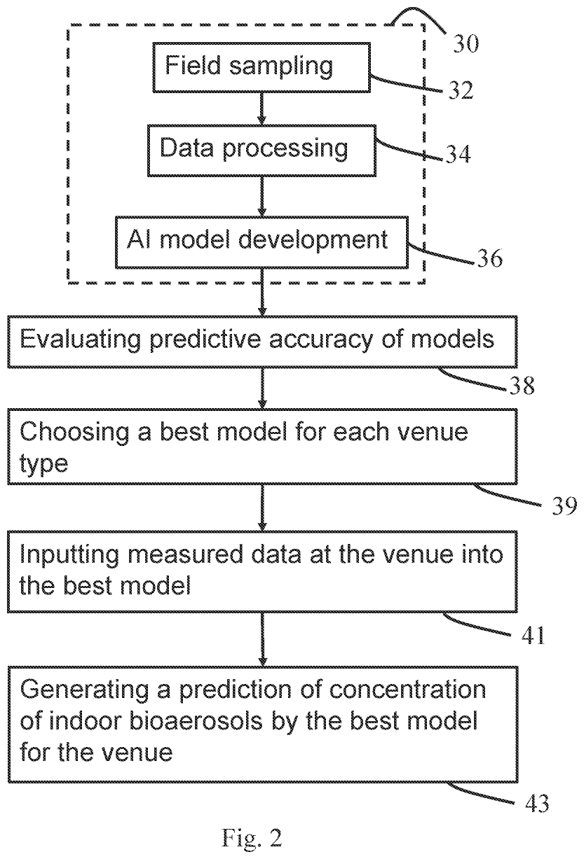

Next, FIG. 2 illustrates the main steps of a method for predicting concentration of indoor bioaerosols. Part of the method can be implemented using the system of FIG. 1 or another similar system. However, it should be noted that the method introduced herein includes further steps of developing a plurality of different AI models, and selecting a best model for each type of indoor venues. The development of AI models involves further hardware not shown in FIG. 1, since FIG. 1 shows the system for making the forecast based on AI models that have already been developed. The method in FIG. 2 starts at Step 30 in which a plurality of artificial intelligence (AI) models are provided prior to selecting a best model for a particular type of venue. Step 30 further include three sub-steps, which are a field sampling step 32, a data processing step 34, and AI model development 36. These steps will be described in details below.

Field Sampling

For the field sampling step 32, two different IAQ sensors are put together to collect necessary IAQ data for the later AI model development 36. In this example, the IAQ data are collected from an operating commercial office and a shopping mall. The measured data from the IAQ sensors are processed and curated prior to input into the AI models as will be described in more details later. The first IAQ sensor could be the IAQ sensor 20 in FIG. 1 (which means that a same sensor 20 is used both for AI model development and actual monitoring/forecasting), or it could be another IAQ sensor different from but being similar to the IAQ sensor 20. Also, there is a second sensor which is an ultraviolet light/laser-induced fluorescence (UV-LIF) real-time bioaerosol cytometer (not shown). The deployment of the UV-LIF real-time bioaerosol cytometer is for the purpose of acquiring the high temporal resolution biological data required for model training, but it should be emphasized that once the AI models have been developed, then for the monitoring and forecasting of the indoor bioaerosols concentration alone, the UV-LIF real-time bioaerosol cytometer is no longer needed. Rather, as mentioned above only an IAQ sensor providing the required physical and chemical properties of indoor air for forecasting is required. In this way, a low-cost compact commercial grade IAQ sensor is sufficient to predict bioaerosols for the user, so this technology can be widely deployed in buildings.

In one implementation, a real-time fluorescence-based aerosol cytometer (InstaScope, Boulder, CO, USA) as the second sensor is operated at a flow rate of 0.85 L/min to identify bioaerosols based on the light-scattering and fluorescence spectra of airborne particles. Three fluorescence channels are used in tandem to classify airborne particles: channel A (excitation=280 nm, emission=310-400 nm), channel B (excitation=280 nm, emission=420-650 nm) and channel C (excitation=370 nm, emission=420-650 nm).¹⁶ A particle with a fluorescence intensity that exceeded the instrument intrinsic noise baseline by three standard deviations was classified into one of the following four fluorescent-type categories: A, AB, BC or ABC.¹⁶ A particle was classified as either bacteria-like, fungi-like or pollen-like by analysis of its fluorescence and optical size properties, according to a previous study¹⁵ (see Table 1 below). Despite the merits of an ultraviolet light/laser-induced fluorescence instrument, there are uncertainties attached to the assignment of fluorescent particles to biological matters.¹⁸ This study refers to bacteria-like, fungi-like, and pollen-like particles as bacteria, fungi, and pollen, respectively, for simplicity in descriptions. A custom R script was used to process the raw data generated. The physical and chemical properties of indoor air are measured using a commercial grade IAQ sensor (Kaiterra Ltd., Mollens, Switzerland) as the first sensor. Six parameters are measured per minute: temperature, RH, concentrations of CO₂, TVOCs, PM_{2.5} and PM₁₀. All instruments are calibrated prior to use.

TABLE 1
Fluorescence threshold of the ultraviolet light/laser-induced fluorescence
instrument and the classification rubrics of particles.
Fluorescence Threshold

| Location | Channel A | Channel B | Channel C |
|---|---|---|---|
| Commercial office (1) | 120.29 | 293.61 | 115.92 |
| Commercial office (2) | 118.08 | 276.32 | 105.90 |
| Commercial office (3) | 118.79 | 290.72 | 110.07 |
| Shopping mall | 113.55 | 293.23 | 90.93 |

Classification Rubric

| Biological matter | Fluorescent Category | Size (μm) |
|---|---|---|
| Bacteria | A | 0.5-1 |
| Fungi | A, AB | 2-9 |
| Pollen | BC, ABC^{a} | 2-10 |

^{a}Category ABC was classified as pollen only, not both pollen and fungi as indicated in Hernandez et al. (2016)¹⁵.

As a practical case, the indoor air of a typical commercial office (from April 26 to May 25, 2021, and from June 2 to 14, 2021) and a shopping mall (from Dec. 20, 2021, to Jan. 12, 2022) in Hong Kong were measured every minute for 24 h a day during the above-stated sampling periods. The commercial office was open-plan, ˜1,400 m² and had ˜250 people sitting in rows of desks without partitions. The staff office hours were 08:30 to 17:30 Monday to Friday and the heating, ventilation and air conditioning (HVAC) systems operated from 07:00 to 19:00 on weekdays. Separate measurements were made in the office at each of three different locations on at least 8 consecutive weekdays. Two sampling locations were close to the middle of the office, with one adjacent to many occupants and the other adjacent to fewer occupants, while the third location was near the back of the office, away from the seating area. The data from the three locations were subsequently combined into a single representative dataset for downstream analysis. In the shopping mall, the store hours were 11:00 to 22:00 7 days a week and the HVAC systems operated from 10:00 to 22:00. Measurements were made at a single location on a floor in a ˜2,000-m² section that housed individual shops and a children's playground for 21 consecutive days spanning weekdays and weekends. At the sampling location, ˜100 people passed by on average per hour and the occupancy on weekends was ˜50% higher than that on weekdays.

Data Processing

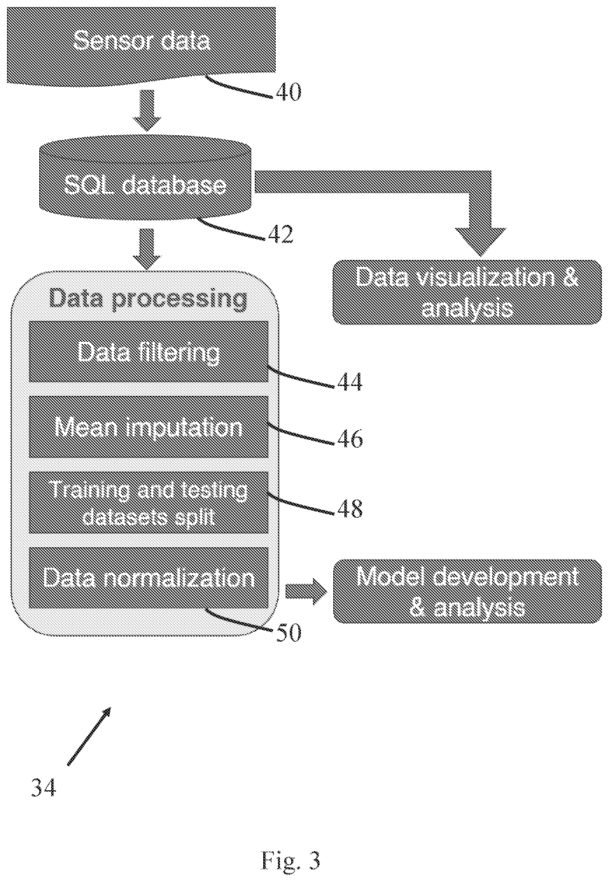

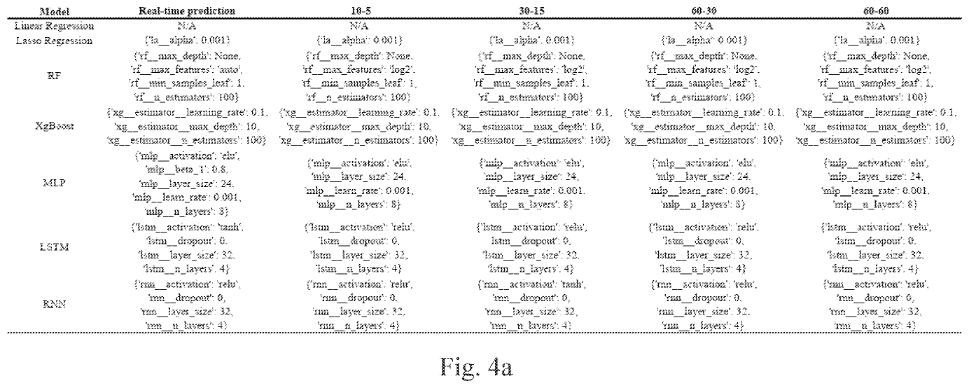

After the measured data from the IAQ sensors are obtained in Step 32, in the data processing step 34 the measured data are processed and curated prior to input into the AI models. FIG. 3 shows detailed steps of the data processing step 34. All of the measured data 40 from the IAQ sensors are tabulated and stored in a Structured Query Language (SQL) database 42 constructed using sqlite⁵³ (v 3.37.0). The measured data are used to determine the IAQ of the two sampling locations and to develop AI models to predict the IAQ. All of the procedures for data visualization and analytics are performed using Python (v 3.6.15). The following four steps are performed to curate the measured data prior to inputting them into an AI model. First, in Step 44 data from non-office and non-store hours are removed from the two datasets. Second, in Step 46 mean imputation was performed to fill any missing values for all of the parameters. This afforded 15,310 and 15,203 time-series data points for the commercial office and shopping mall, respectively. Third, in Step 48 an AI model (i.e. one of the seven models as will be mentioned below) was allocated the data for six parameters (temperature, RH, and the concentrations of CO₂, TVOCs, PM_{2.5} and PM₁₀) as input features and the data for five parameters (the concentrations of bacteria, fungi, pollen, PM_{2.5} and PM₁₀) as target features. The data points are randomly split into training and testing datasets at a ratio of 9:1. For testing different input and output time windows (see below), the data are grouped according to the length of time windows and then randomly allocated into the training or testing datasets. Fourth, in Step 50 the values for the input and target features are normalized (i.e., from 0 to 1) to eliminate potential biases.

Development of AI Models

After the measured data has been processed, the method in FIG. 2 goes to Step 36 in which a plurality of AI models are developed. In one implementation, a Python environment (v. 3.6.15) was used for the development and evaluation of AI models. Six parameters are set as input features for each AI model: temperature, RH and the concentrations of CO₂, TVOCs, PM_{2.5} and PM₁₀. Seven AI models are built—a linear regression model,²³ a lasso regression model,^{24,25} a random forest (RF) model,²⁶ an extreme gradient boosting (XgBoost) model,²⁷ a multilayer perceptron (MLP) model,²⁸ an LSTM model^{29,30} and a recurrent neural network (RNN) model³⁰—to determine the real-time or near-future concentrations of five target features: bacteria, fungi, pollen, PM_{2.5} and PM₁₀. The performance of each AI model was assessed using five combinations of time windows of measured data as inputs and of near-future data as outputs, namely (i) real-time prediction, (ii) a 10-min input and a 5-min output (abbreviated as “10-5”), (iii) a 30-min input and a 15-min output (“30-15”), (iv) a 60-min input and a 30-min output (“60-30”) and (v) a 60-min input and a 60-min output (“60-60”). Each combination contains a pair of input and output time windows, and for (i) real-time prediction the pair is a real-time window pair, i.e., the input and output time windows are both approximately zero.

With more details, in the implementation, model training and evaluation are all conducted using Python (v. 3.6.15) on a typical workstation computer (OS: Ubuntu 20.10, CPU: Intel® Xeon® E-2136 with 64 GB of memory) in a Linux environment. Four machine learning models are developed: three models (a linear regression model,²³ a lasso regression model^{24,25} and a RF²⁶ model) are developed using the scikit-learn³² package (v 0.24.2), while one model (an extreme gradient boosting (XgBoost)²⁷ model) is developed using the package xgboost²⁷ (v 1.4.0). The linear regression and lasso regression models are linear models; lasso regression model performs L1-regularization to penalize the magnitude of coefficients.²⁵ The RF and XgBoost models are non-linear tree-based models; every node of a tree is labeled with a criterion generated based on the input feature that leads to the subordinate decision nodes of another criterion, while each leaf of a tree returns a regression value as an output.

Three deep learning models (a MLP model,²⁸ a LSTM model^{29,30} and a RNN model³⁰ are developed using Keras⁵⁴ (v 2.3.1) in the Python package TensorFlow⁵⁵ (v 1.14) and optimized using the Adam optimizer.⁵⁶ All three models are composed of three types of layers: input, hidden and output layers. The hidden layers in an MLP model consist of several nodes, with each being responsible for computing a mathematical function. The MLP model outputs are defined by a weighted calculation between all of its neural nodes. A similar hidden layer is also present in an LSTM model and an RNN model; however, an LSTM model also contains a specific layer to maintain weighted past records in memory for a long period of time, whereas an RNN model contains a specific layer to maintain the last input parameters in memory.

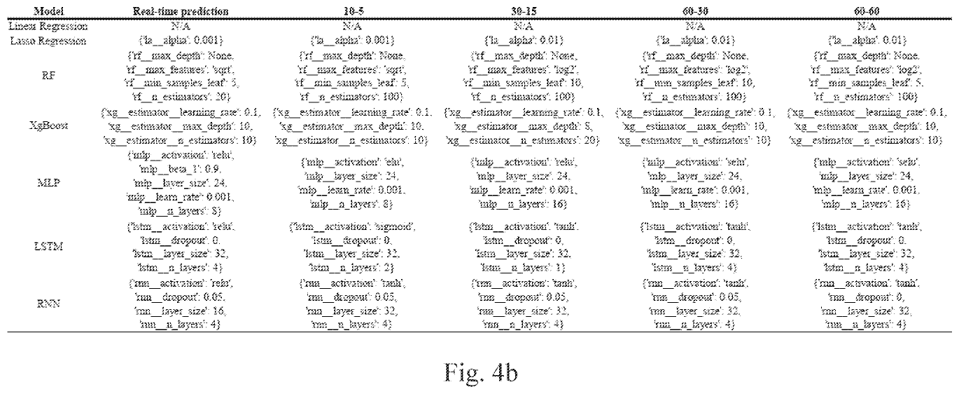

The sets of hyperparameters (see Table 2 below) for each model were optimized during model training by a grid search⁵⁷ function with five-fold cross validation provided by the scikit-learn package. This function iterated all combination sets of hyperparameters to configure and train each model and thus returned an optimized model with a set of hyperparameters (see FIGS. 4a-4b) that rendered the lowest loss value as determined by the mean squared error. In addition to the hyperparameters listed in FIGS. 4a-4b, the batch size and the number of epochs for the three deep learning models were set at 32 and 100, respectively. To enable the models to simultaneously forecast the concentrations of the five target features, a multi-output regression function in the scikit-learn package was also applied.

TABLE 2
The set of hyperparameters that were tuned by
the grid search method for the seven AI models.

| Model | Hyperparameters |
|---|---|
| Linear Regression | N/A ^{a} |
| Lasso Regression | Alpha: [0.001, 0.01, 0.1, 0.5] |
| RF | Number of Estimators: [5, 10, 20, 50, 100] |
| | Maximum Depth: [2, 6, None] |
| | Maximum Features: [“auto”, “sqrt”, “log2”] |
| | Minimum Samples Leaf: [1, 2, 5, 10] |
| XgBoost | Number of Estimators: [10, 20, 50, 100] |
| | Maximum Depth: [5, 8, 10, None] |
| | Learning Rate: [0.001, 0.01, 0.1, 1] |
| | Learning Rate: [0.001, 0.01, 0.1] |
| MLP | Beta 1^{b}: [0.9, 0.8, 0.7] |
| | Activation Function: [“relu”, “selu”, “elu”] |
| | Number of Dense Layers: [2, 4, 8, 16] |
| | Layer Size: [8, 16, 24] |
| LSTM | Dropout Rate: [0, 0.05, 0.1] |
| | Activation Function: [“tanh”, “sigmoid”, “relu”] |
| | Number of LSTM Layers: [1, 2, 4, 8] |
| | Layer Size: [8, 16, 32] |
| RNN | Dropout Rate: [0, 0.05, 0.1, 0.3] |
| | Activation Function: [“tanh”, “sigmoid”, “relu”] |
| | Number of RNN Layers: [1, 2, 4] |
| | Layer Size: [8, 16, 32] |

^{a} N/A: Not applicable
^{b}A hyperparameter for the Adam optimizer. This hyperparameter was optimized only in the model training for real-time prediction, the default value (0.9) was used for the other time windows.

In addition, five different combinations of time windows (each combination has a pair of input and output time windows) of past measured data to be used as input features and time windows of predicted data for the target features are investigated. The time window of past measured data constrained how far back into the past the values for input features (including the current moment) should be obtained for use in forecasting, while the time window of future data constrained how far into the future values of the target features are forecasted. To obtain accurate predictions, a time window that was longer than or the same length as the output data was adopted for the input data. The five different combinations of time windows tested are (i) real-time prediction, (ii) a 10-min input and a 5-min output (abbreviated as “10-5”), (iii) a 30-min input and a 15-min output (“30-15”), (iv) a 60-min input and a 30-min output (“60-30”) and (v) a 60-min input and a 60-min output (“60-60”). As an example, for “10-5,” the past 9 min, including the current minute (i.e., 10 min in total), of measured input features are used to forecast the target features in the subsequent 5 min. For real-time prediction, the measured real-time input features are used to forecast the target features at the same moment in time. Data for both the measured input and predicted target features are set to have a time interval of 1 min.

Evaluation of Predictive Accuracy of Models

In FIG. 2, after the plurality of AI models have been developed, the method goes to Step 38 in which the prediction accuracy of each of the plurality of AI models for a particular type of venue is evaluated. Consequently, a best model from the plurality of AI models can be chosen for the particular type of indoor venue.

In particular, the difference between the measured values and those predicted by each model were evaluated to determine each model's predictive accuracy, in terms of its MSE, RMSE and/or value on a revised version of the WI³¹. If a model has the best difference data as compared to other models, then the model is determined as the best model for the given type of indoor venue. The MSE and RMSE are computed using the package scikit-learn³² (v 0.24.2), and the WI value (between 0 and 1, with a higher value indicating a more accurate prediction) was determined, using the following equations respectively:

$$WI = 1 - \frac{{\sum }_{i = 1}^{n} \vert {y}_{i} - {\hat{y}}_{i} \vert}{{\sum }_{i = 1}^{n} ( \vert {y}_{i} - \hat{y} \vert + \vert {\hat{y}}_{i} - \overline{y} \vert )} \\ MSE = \frac{1}{n} {\sum }_{i = 1}^{n} {\vert {y}_{i} - {\hat{y}}_{i} \vert}^{2} \\ RMSE = \sqrt{\frac{1}{n} {\sum }_{i = 1}^{n} {( {y}_{i} - {\hat{y}}_{i} )}^{2}}$$

where n is the total number of data points, ŷᵢ is the i-th predicted value, yᵢ is the i-th measured value and y̅ is an average of the measured values.

Unlike the original WI, which squares the errors prior to summation, the revised WI does not over-weight the influence of errors on the sum-of-squared errors³¹ and thus is less sensitive to errors concentrated in outliers and can better differentiate well-performing models.³¹ A custom script was used to calculate the revised WI.

In addition, all statistical analyses are performed using Python. Pairwise Pearson's correlations between IAQ parameters were calculated using the package Scipy³³ (v 1.5.2). Linear regressions of the measured and predicted values were computed using the package statsmodel³⁴ (v 0.12.0). Permutation importance,³⁵ which represents the importance of each input feature in an AI model, was analyzed using the package scikit-learn with the default number of permutations.

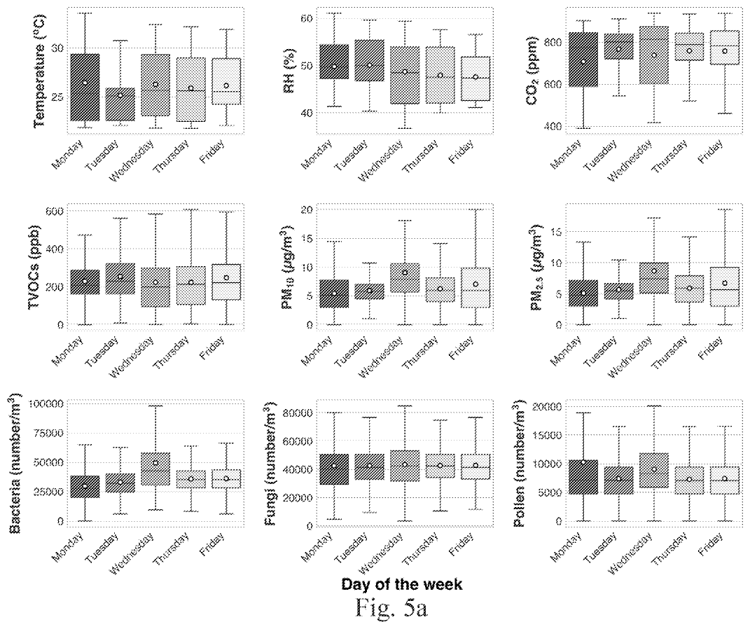

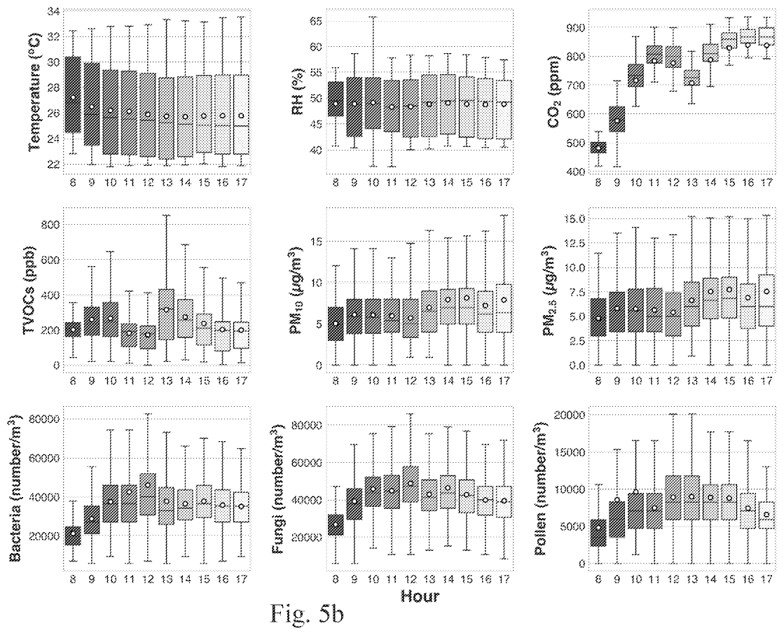

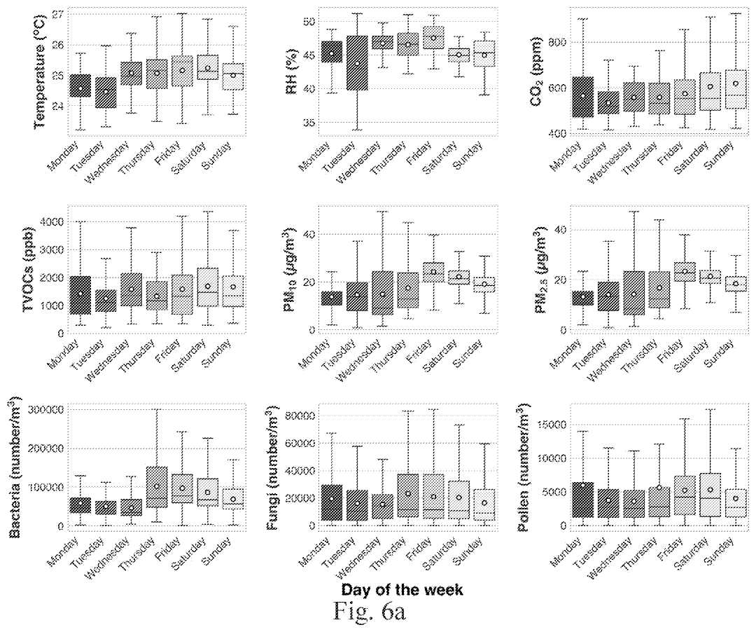

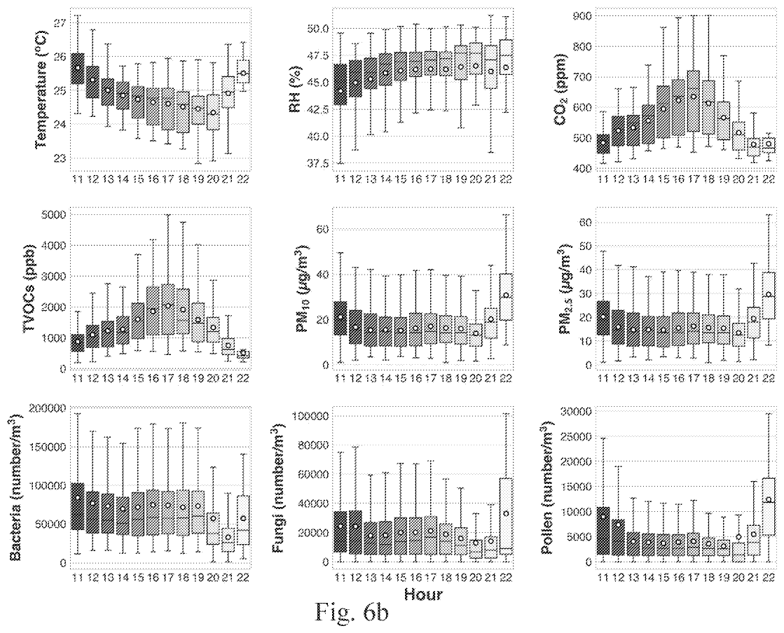

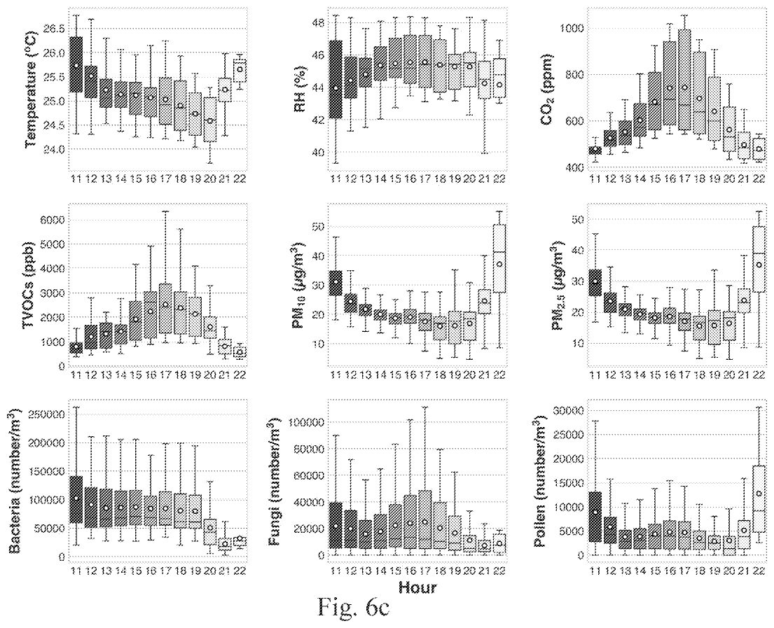

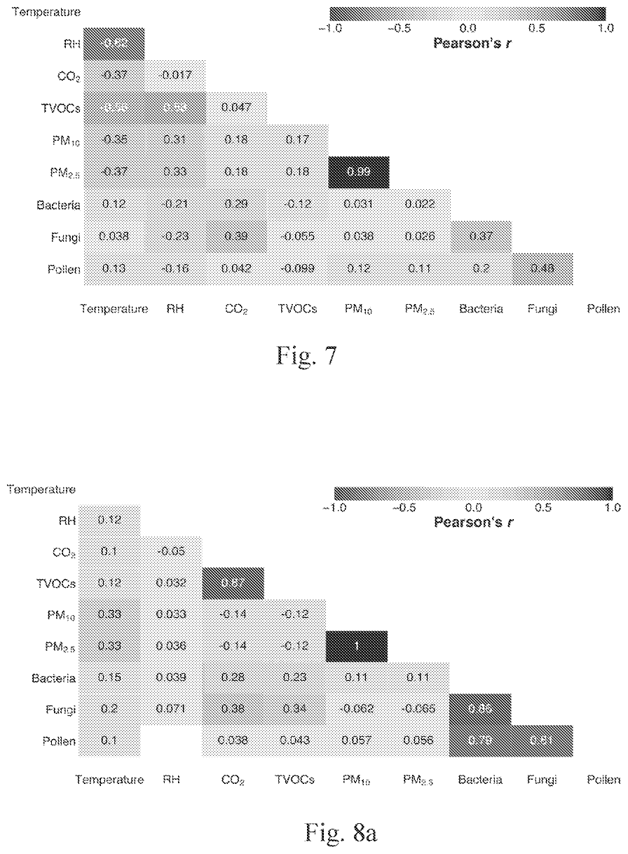

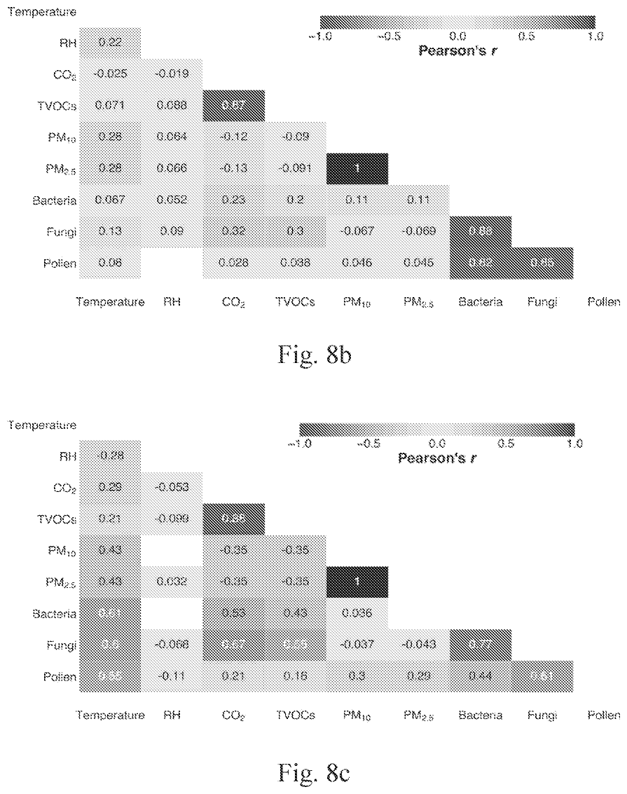

The IAQ of the two venues in the example (i.e. the commercial office and the shopping mall) are now discussed. The daily and hourly average values of nine parameters were analyzed to assess the physical, chemical and biological profiles of indoor air in a commercial office (FIGS. 5a-5b) and a shopping mall (FIGS. 6a-6c). In FIGS. 5a-5b and 6a-6c, the mean (shown as the circle in each box), median (middle horizontal line), as well as 25th and 75th percentiles (lower and upper ends of the box) are shown. The whisker on each of a boxplot indicates the 1.5 times interquartile range beyond the 25th or 75th percentile. Data for the shopping mall were separated into weekday and weekend sets, given the higher occupancy on weekends. Daily and hourly variations were observed in the values of the nine parameters for each venue, which reflected their operation (e.g., increased concentrations of CO₂ during office hours and decreases during lunch hours in the commercial office). A pairwise Pearson's correlation analysis of the nine IAQ parameters showed statistically significant correlations between most of the parameters for the commercial office (see FIG. 7) and the shopping mall, with all data from weekdays and weekends combined (see FIG. 8a) or separated (see FIGS. 8b-8c). In FIGS. 7 and 8a-8c, all of the correlations are statistically significant (p<0.05).

Performance of the AI Models in Predicting IAQ, and Choosing a Best Model

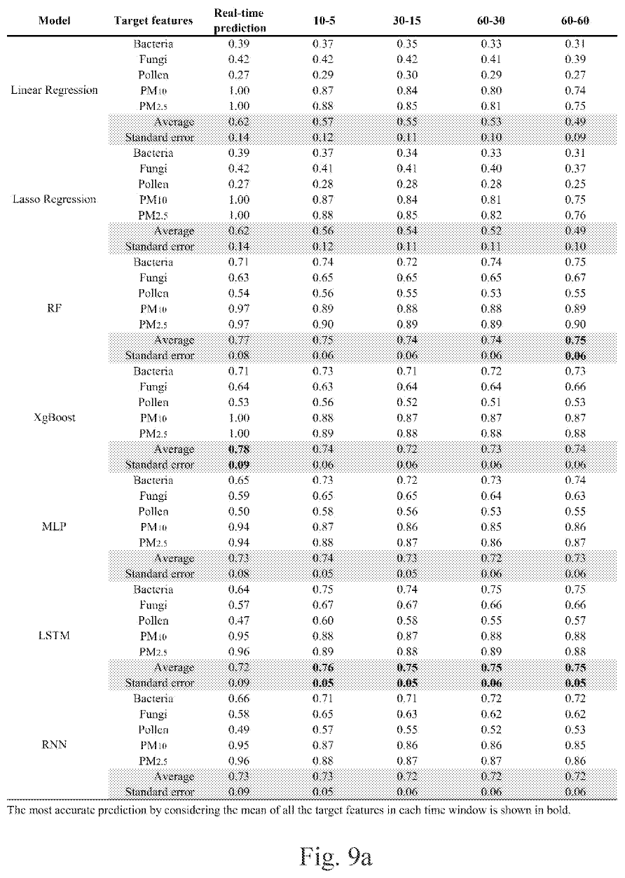

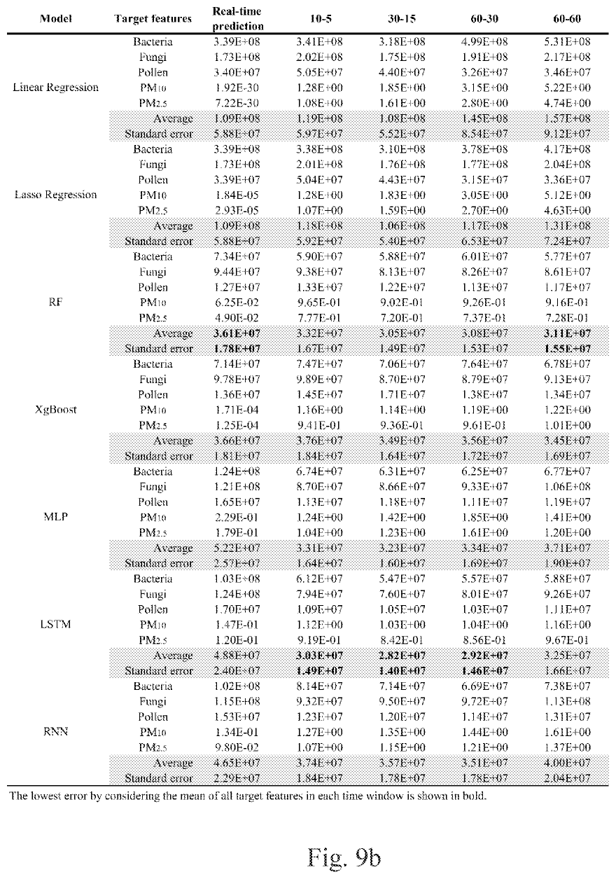

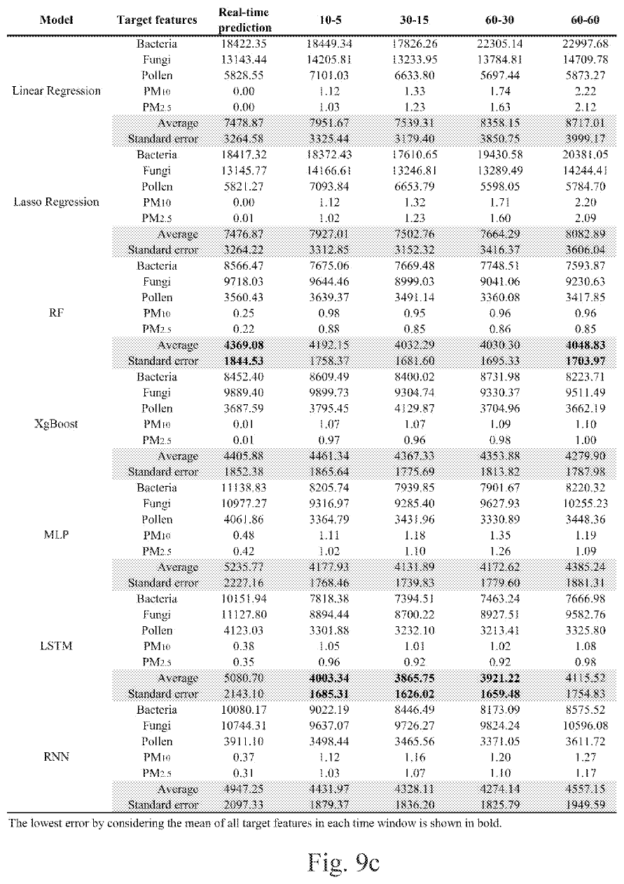

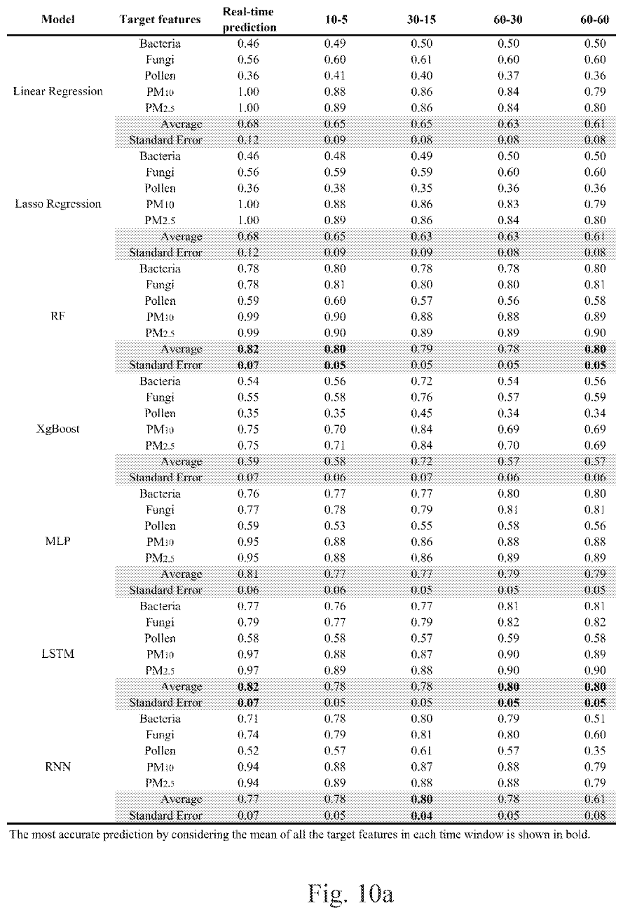

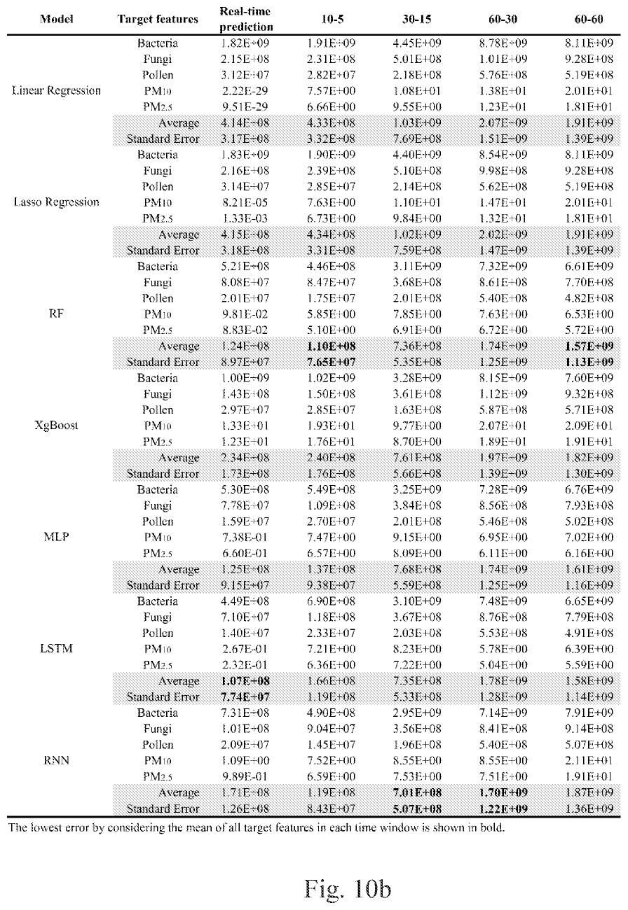

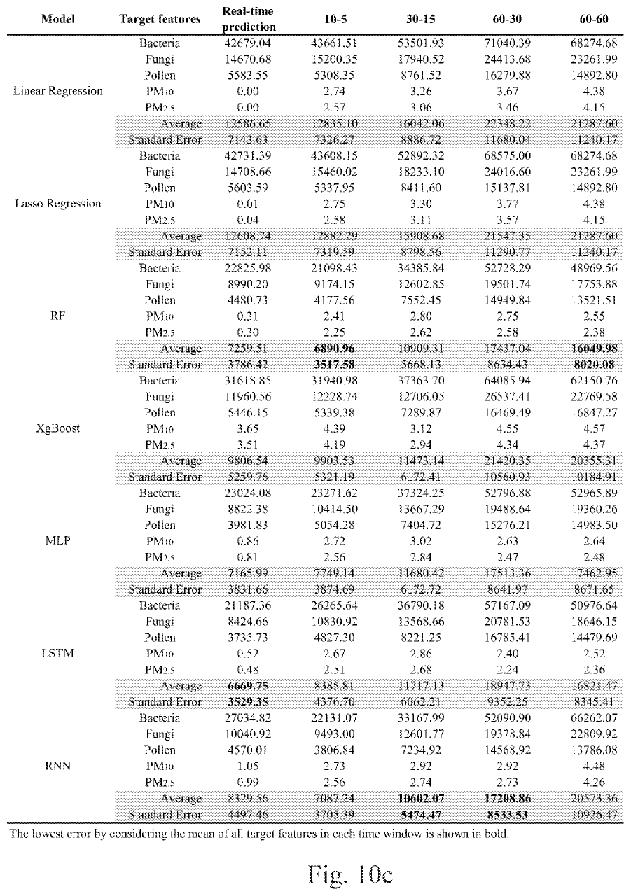

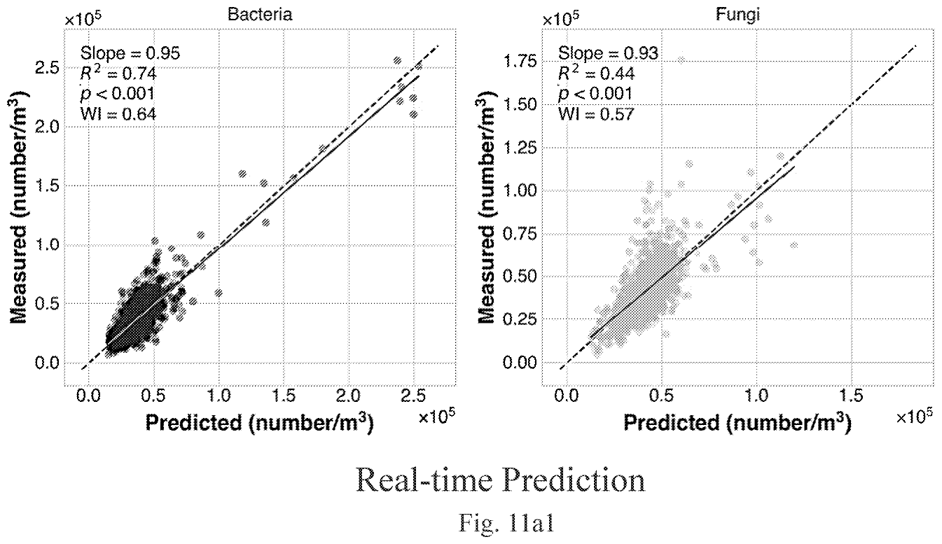

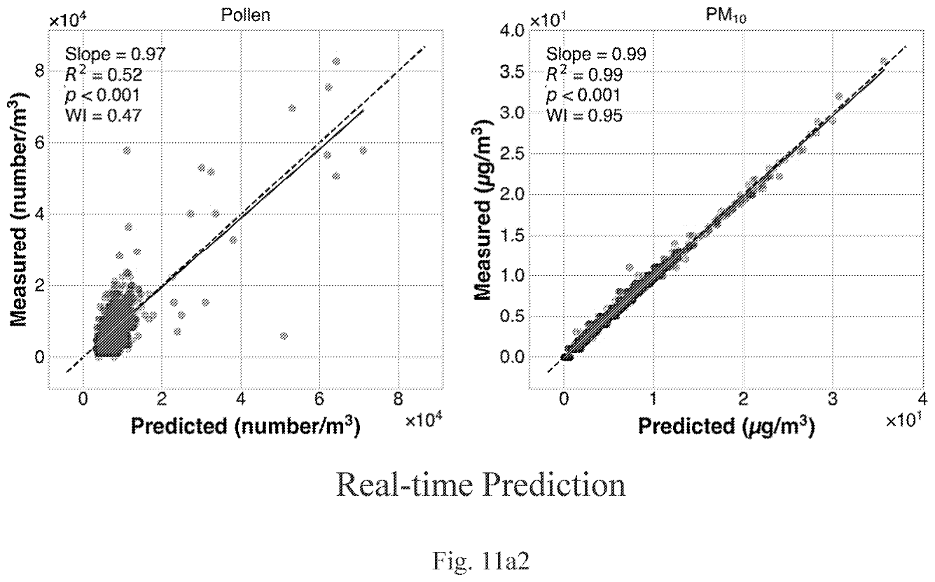

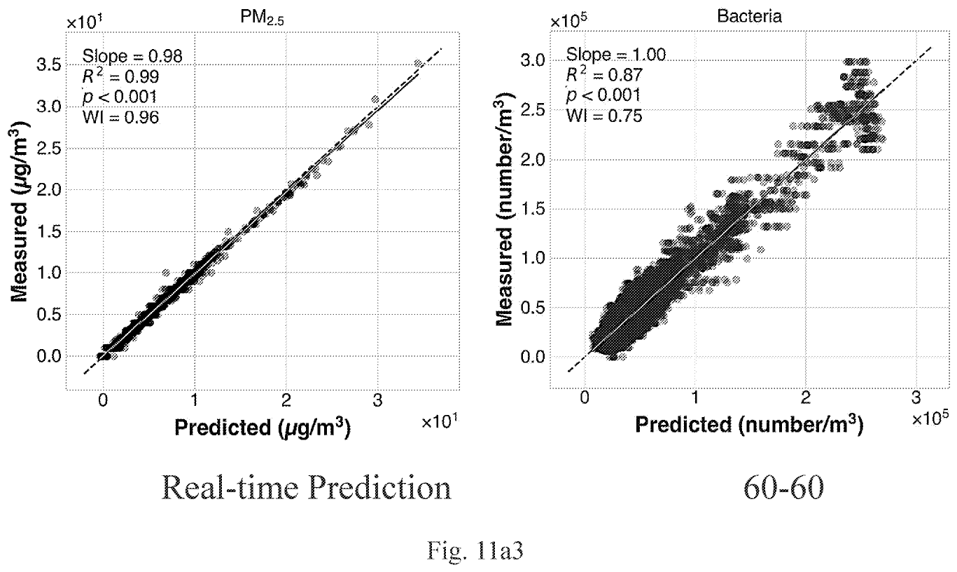

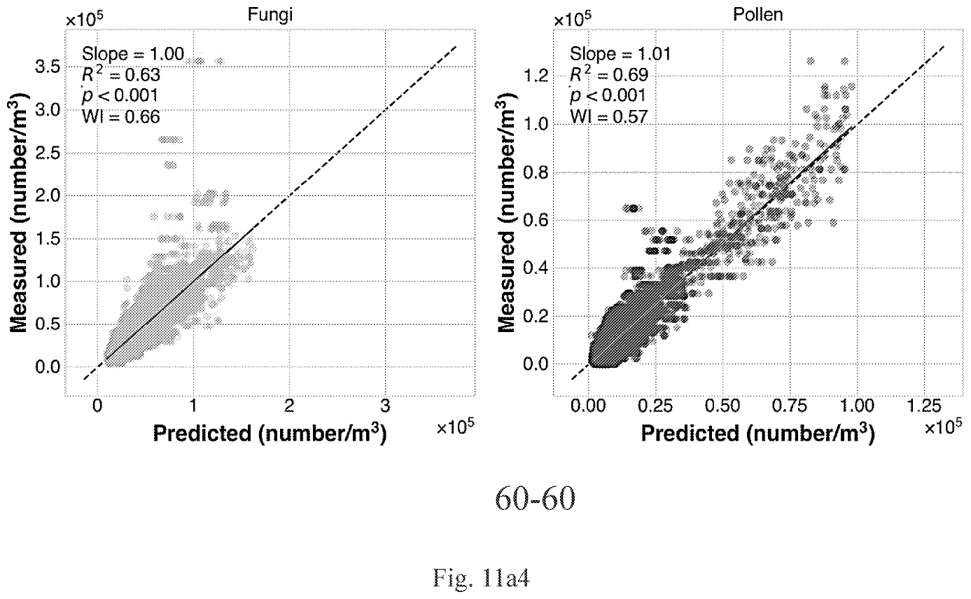

The analysis result of the predictive accuracy of models are now described. The ability of the AI models to determine the target features in various future time windows from various time windows of measured data is evaluated using testing datasets based on the WI, MSE and RMSE (see FIGS. 9a-9c and 10a-10c) and the time required for model training and hyperparameter searching (See Table 3 below).

TABLE 3
The time required for model training and hyperparameter
searching for the commercial office and shopping mall.

| | Real-time | | | | |
|---|---|---|---|---|---|
| Model | prediction | 10-5 | 30-15 | 60-30 | 60-60 |

a) For commercial office

| | | | | | |
|---|---|---|---|---|---|
| Linear | 7.21 ms | 67 ms | 96.7 ms | 228 ms | 314 ms |
| Regression | | | | | |
| Lasso | 840 ms | 39.1 s | 6 min 35 s | 26 min 18 s | 54 min 30 s |
| Regression | | | | | |
| RF | 52.2 s | 7 min 53 s | 43 min 41 s | 2 h 11 min 4 s | 3 h 58 min 23 s |
| XgBoost | 7 min 16 s | 2 h 39 min 11 s | 16 h 52 min 33 s | 2 d 6 h 6 min 59 s | 4 d 13 h 27 min 57 s |
| MLP | 2 h 26 min 29 s | 49 min 49 s | 52 min 33 s | 55 min 13 s | 59 min 13 s |
| LSTM | 2 h 19 min 48 s | 2 h 18 min 25 s | 2 h 20 min 6 s | 2 h 23 min 12 s | 2 h 24 min 49 s |
| RNN | 1 h 4 min 19 s | 1 h 4 min 18 s | 1 h 5 min 14 s | 1 h 7 min 42 s | 1 h 9 min 40 s |

b) For shopping mall

| | | | | | |
|---|---|---|---|---|---|
| Linear | 57.8 ms | 165 ms | 438 ms | 978 ms | 1.55 s |
| Regression | | | | | |
| Lasso | 866 ms | 33 s | 4 min 38 s | 18 min 53 s | 37 min 40 s |
| Regression | | | | | |
| RF | 53.9 s | 7 min 56 s | 45 min 56 s | 2 h 22 min 22 s | 4 h 23 min 15 s |
| XgBoost | 7 min 29 s | 2 h 45 min 37 s | 17 h 48 min 58 s | 2 d 10 h 27 min 12 s | 4 d 21 h 9 min 20 s |
| MLP | 2 h 25 min 28 s | 50 min 58 s | 54 min 13 s | 55 min 58 s | 1 h 1 min 41 s |
| LSTM | 2 h 18 min 16 s | 2 h 20 min 2 s | 2 h 22 min 55 s | 2 h 25 min 39 s | 2 h 30 min 9 s |
| RNN | 1 h 3 min 19 s | 1 h 5 min 55 s | 1 h 8 min 8 s | 1 h 8 min 40 s | 1 h 12 min 29 s |

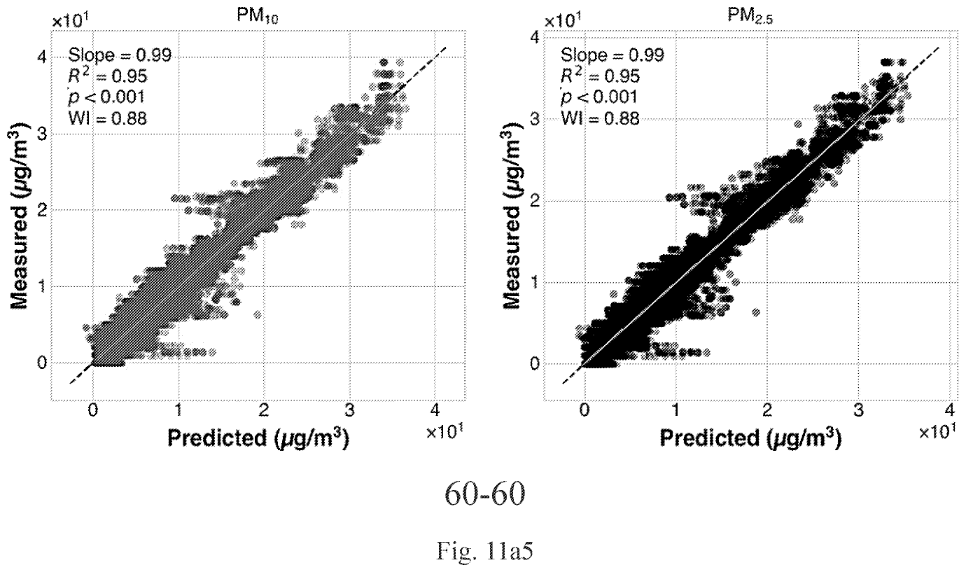

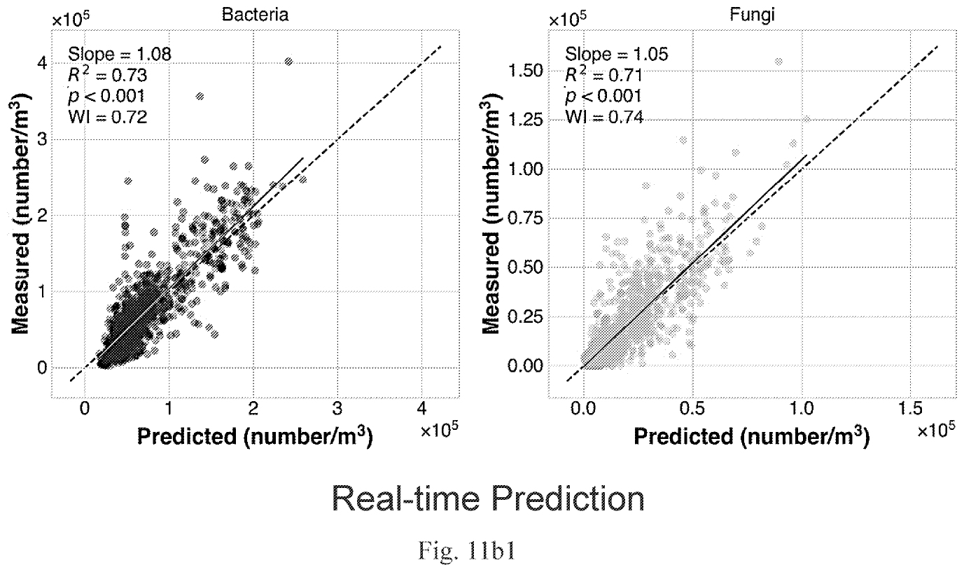

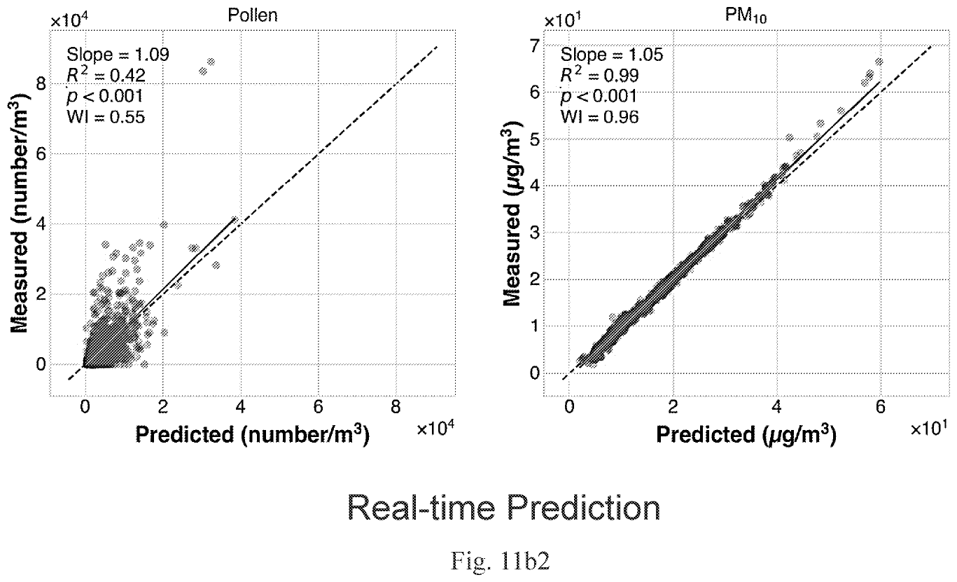

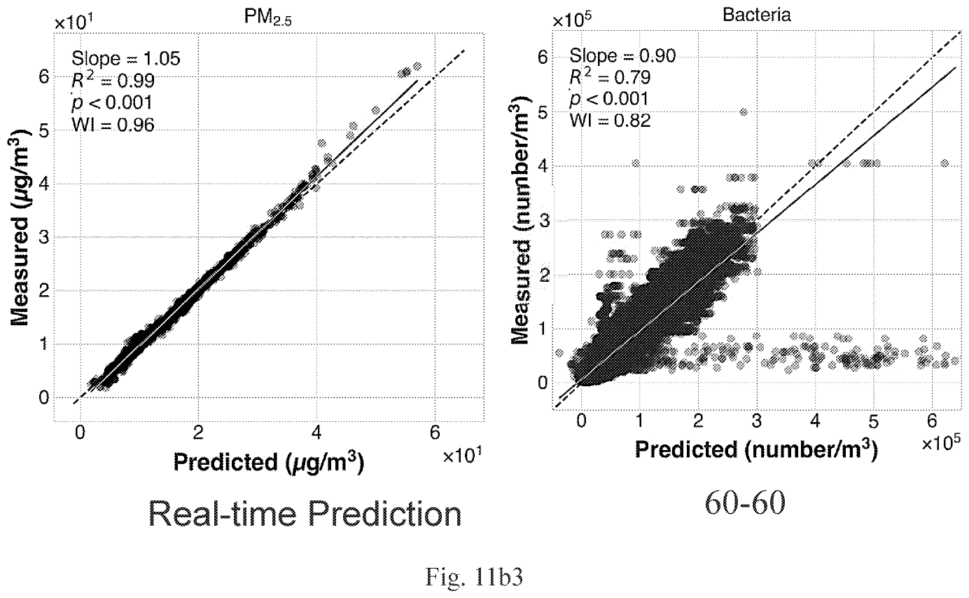

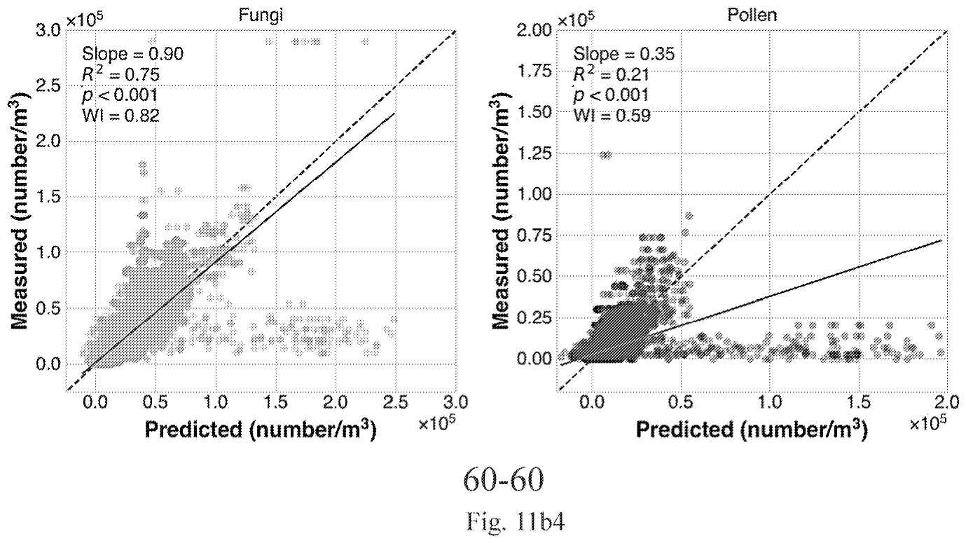

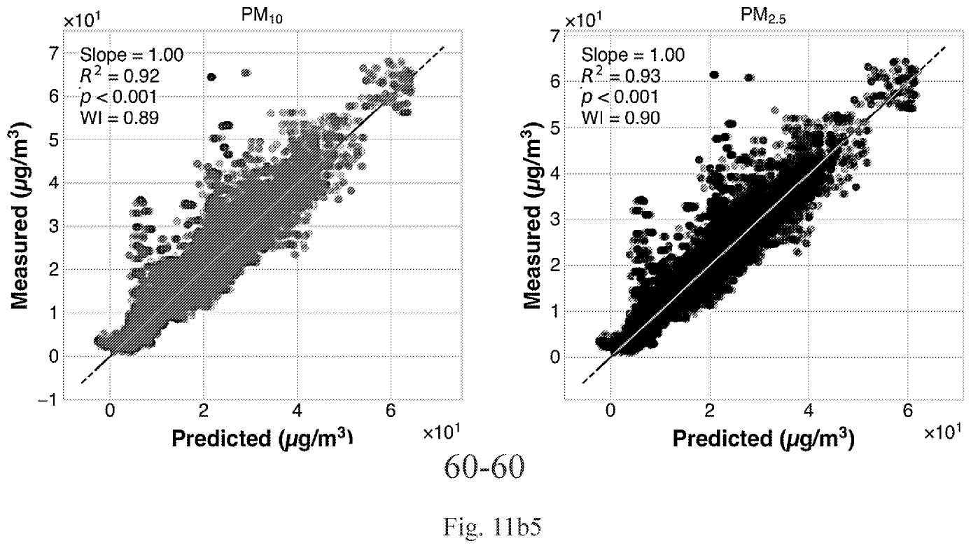

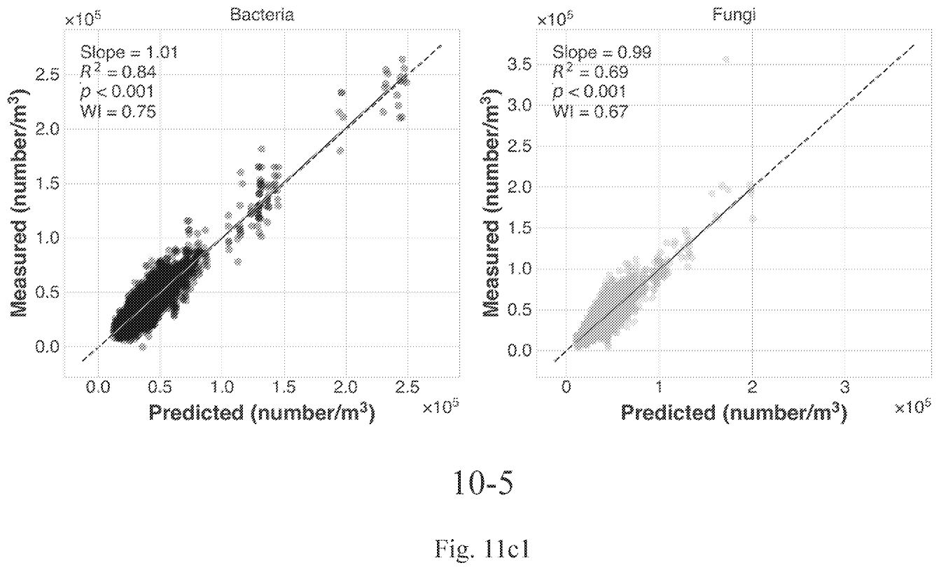

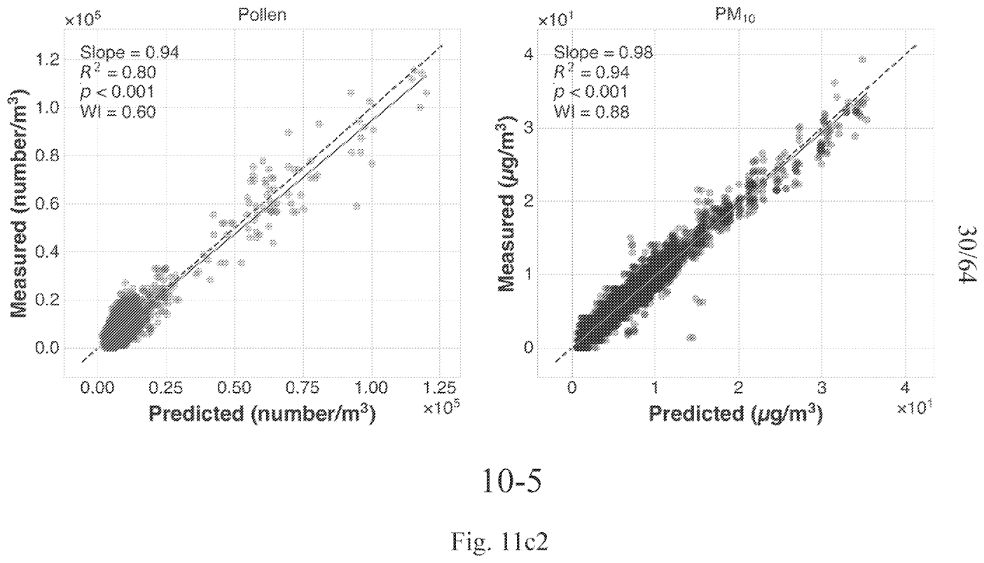

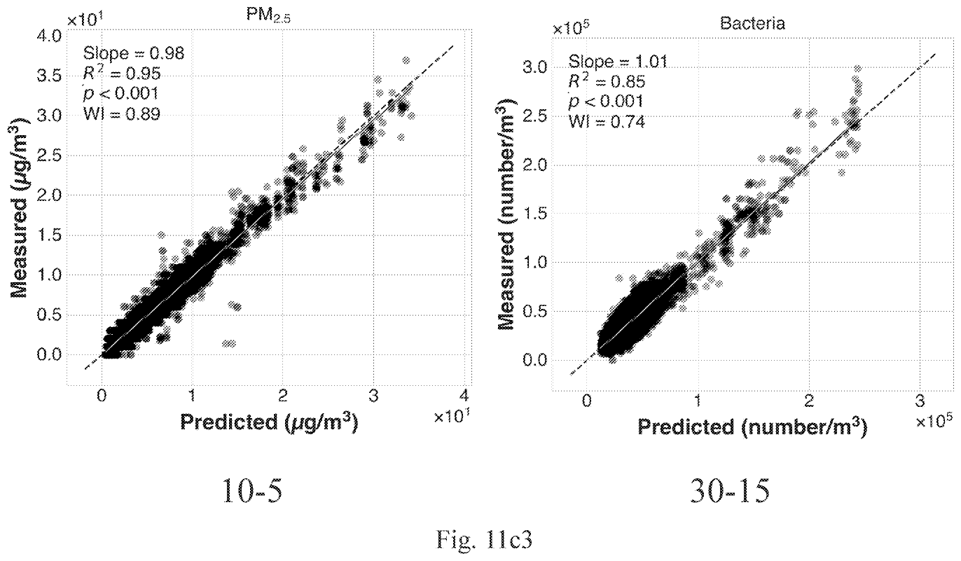

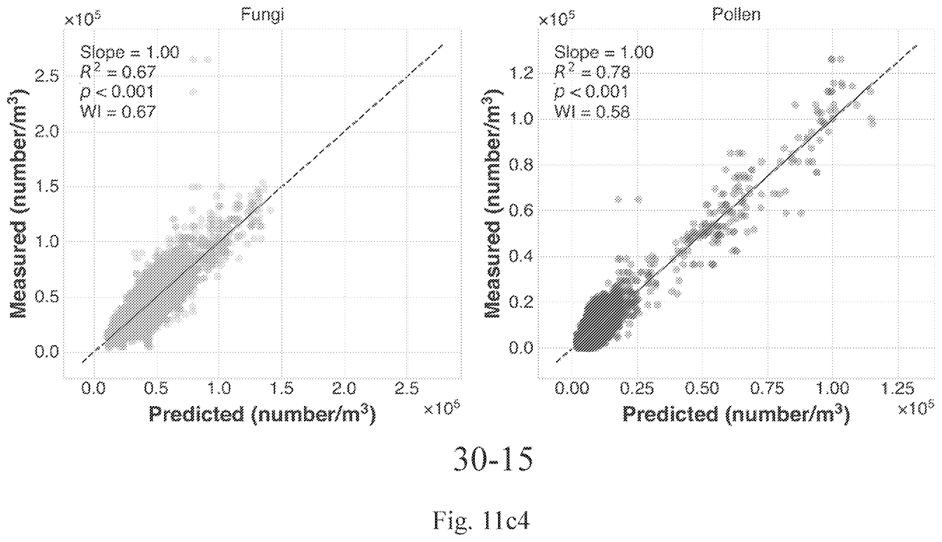

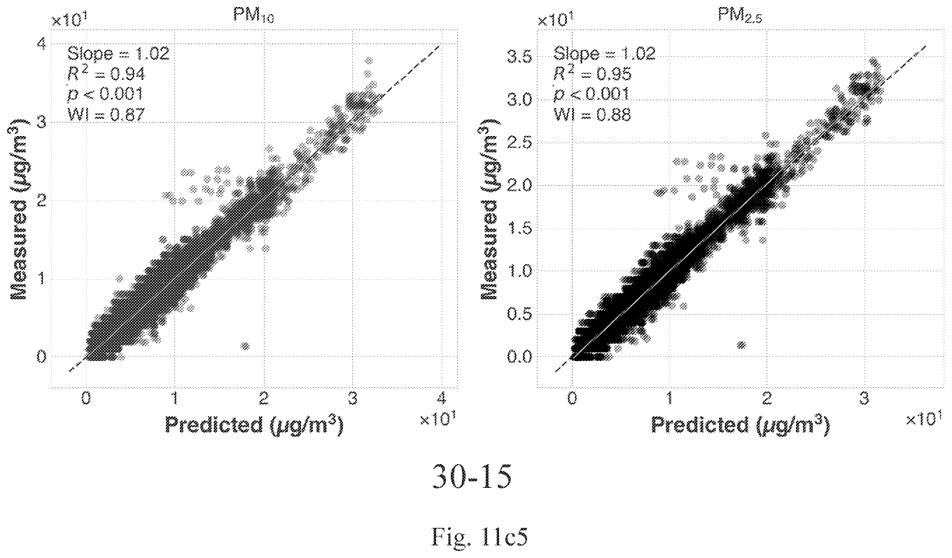

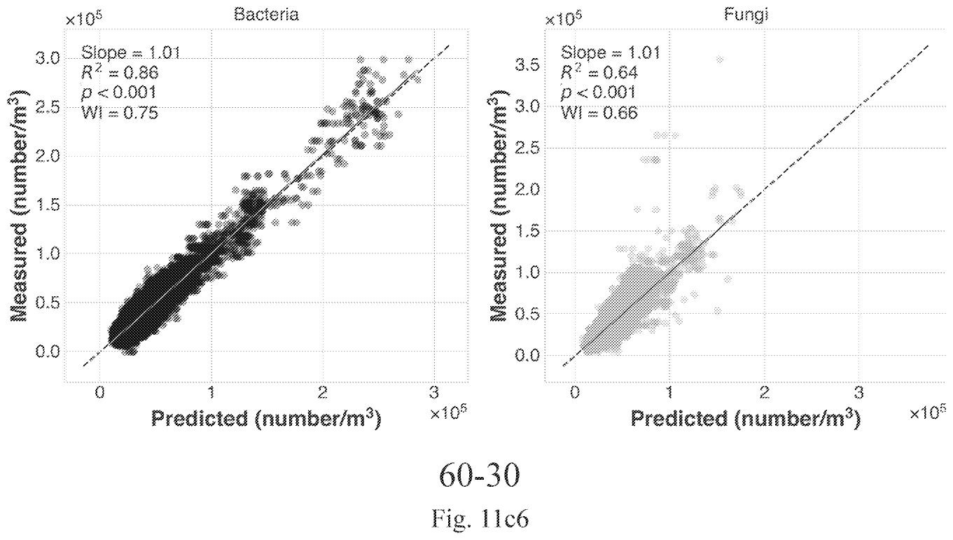

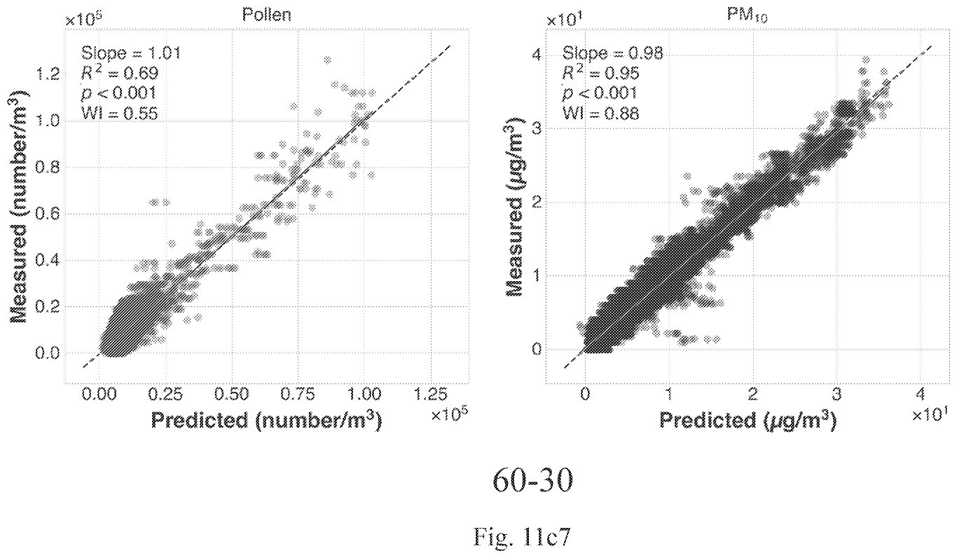

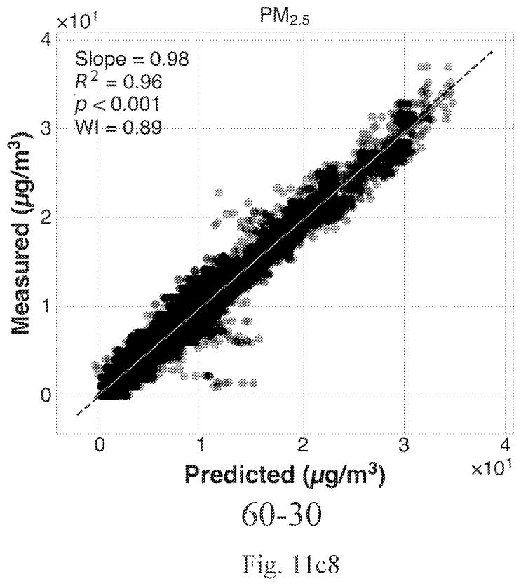

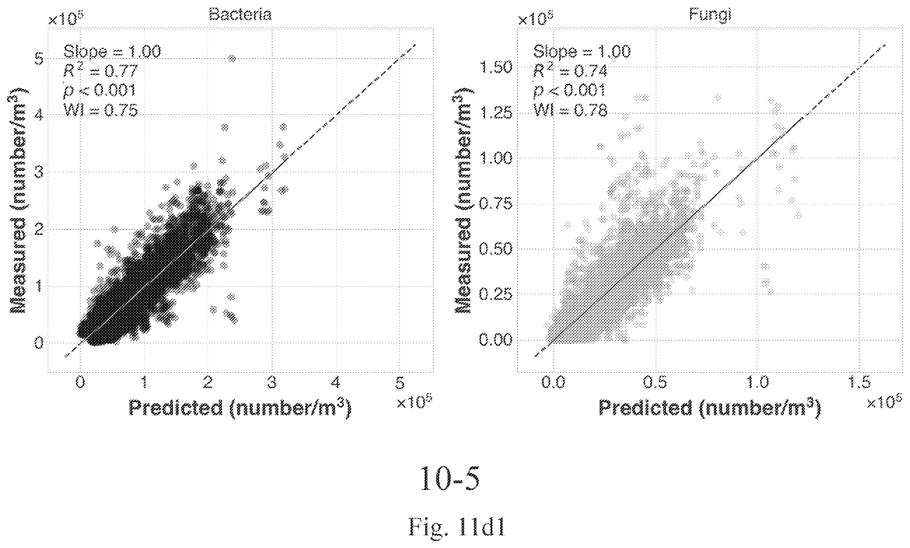

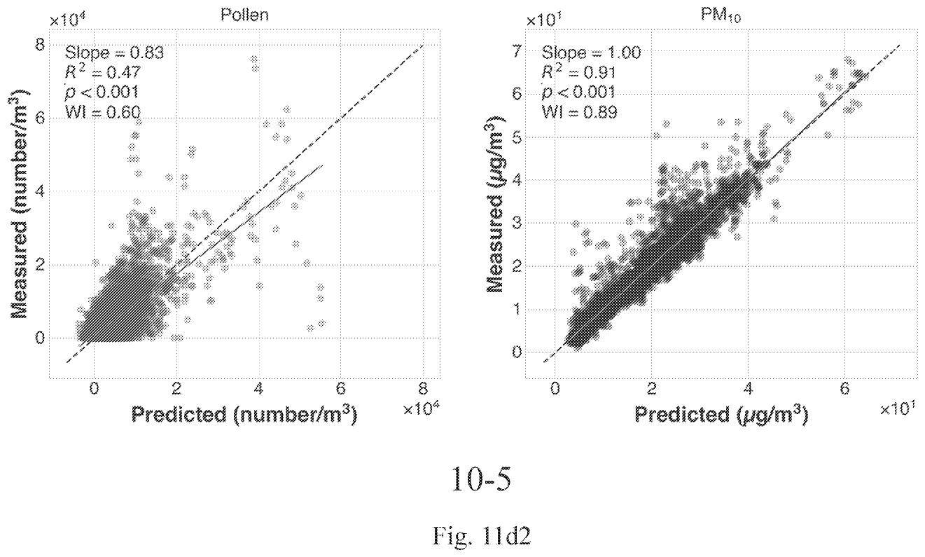

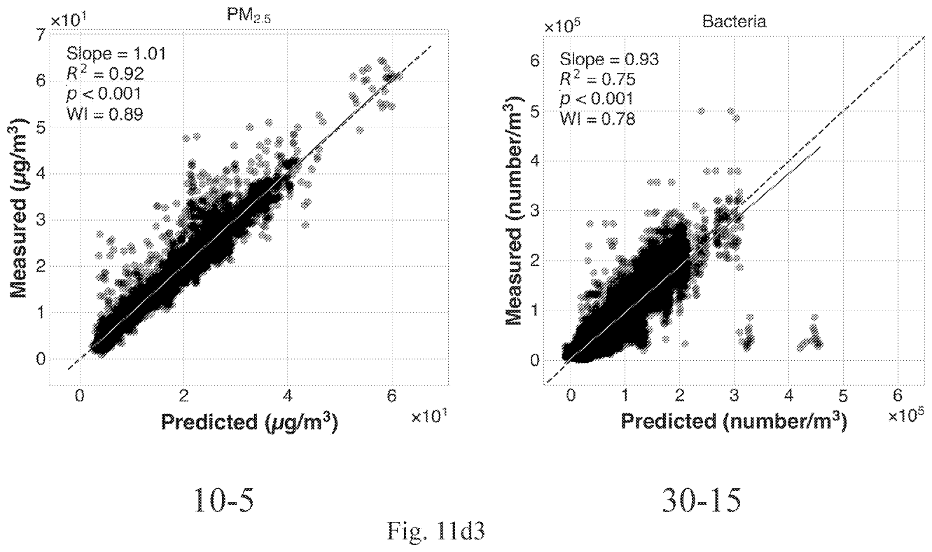

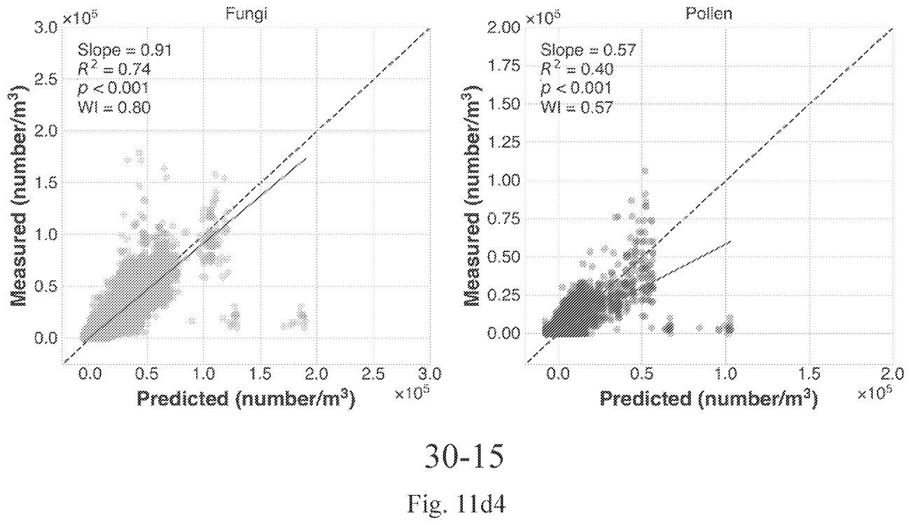

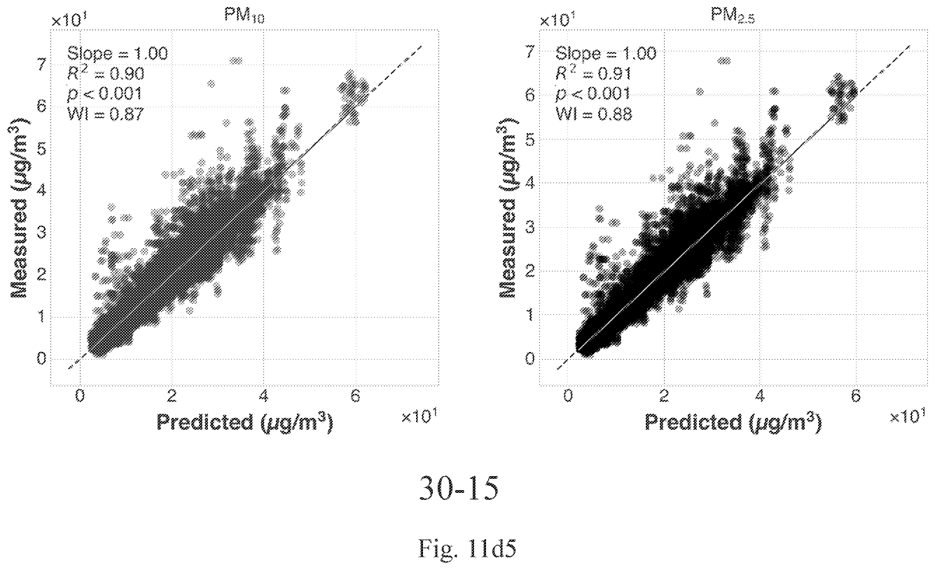

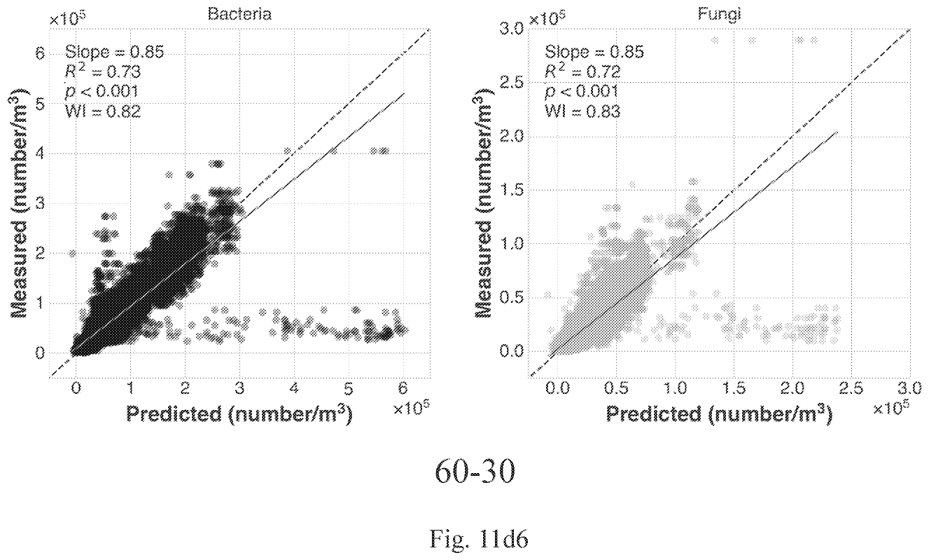

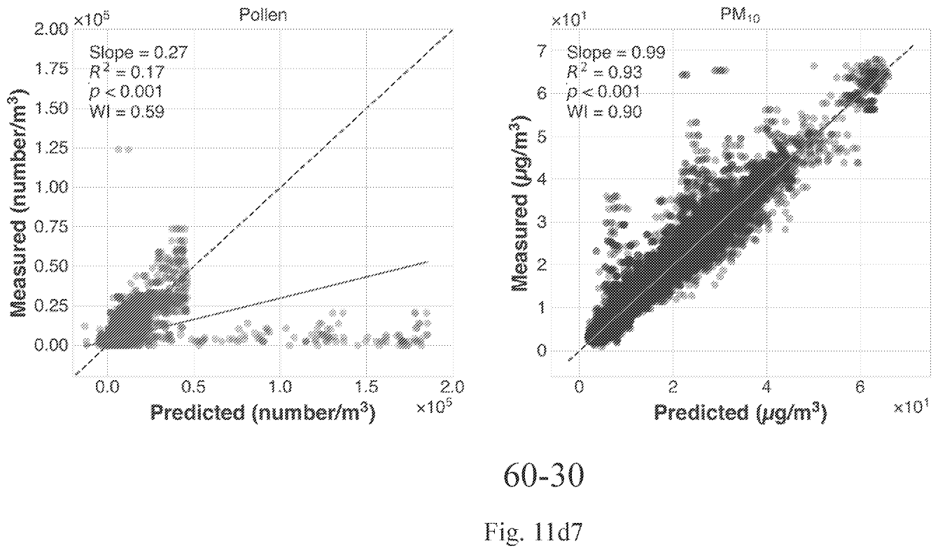

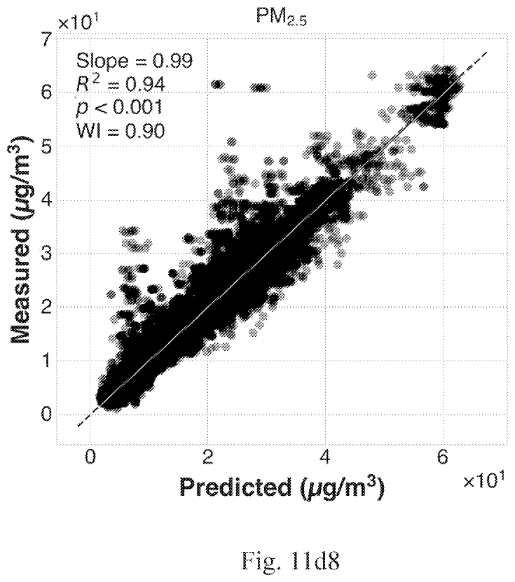

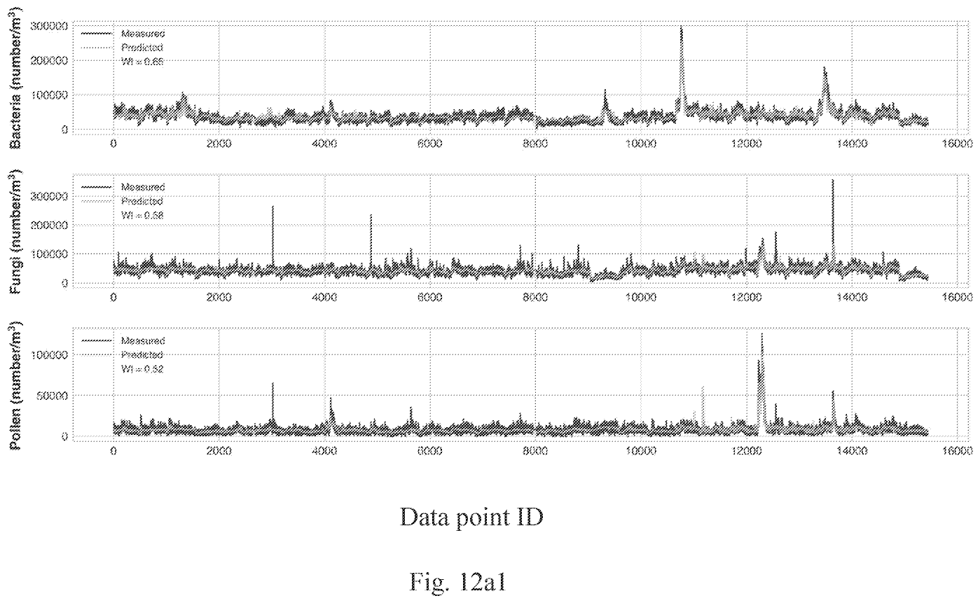

For the commercial office, the predictive accuracy of the linear models—the linear regression and lasso regression models—was poor, regardless of the time windows of data, with an average WI consistently less than 0.62 (see Table 4 below). In contrast, all of the non-linear models exhibited superior performances, with the LSTM model having the highest average predictive accuracy for all of the target features (WI=0.75-0.76) in three of the five time windows tested (i.e., 10-5, 30-15 and 60-30) and the Xgfloost and RF models generating the most accurate values for the real-time prediction (WI=0.78) and 60-60 (WI=0.75) time windows, respectively. Similarly, for all of the target features, the LSTM model had the lowest average MSE and RMSE in the 10-5, 30-15 and 60-30 time windows, and the RF model had the lowest average MSE and RMSE for the real-time prediction and 60-60 time windows (see FIGS. 9a-9c). The accuracy of the prediction made by the LSTM model for each of the target features in the five time windows was further confirmed by the slope of the linear regression of the measured and predicted values being ˜1.0 (although the goodness-of-fit—the value of the coefficient of determination—indicates some data scattering) (see FIG. 11a1-11a5 and FIG. 11c1-11c8). As the length of the time window increased, the average time required for the XgBoost and RF models for training and hyperparameter searching ranged from a few minutes (˜7 min and 1 min, respectively) to hours or days (˜4 d and ˜4 h, respectively), but that required for the LSTM model was similar for all five time windows (˜2.5 h) (see Table 3). Based on the above, in Step 39 (see FIG. 2) the LSTM model is determined as the best model for the venue type of commercial office in this example.

TABLE 4
The average revised WI value of each AI model for different time windows.

| | Time window | | | | |
|---|---|---|---|---|---|
| | Real-time | | | | |
| Model | prediction | 10-5 | 30-15 | 60-30 | 60-60 |

Commercial office

| | | | | | |
|---|---|---|---|---|---|
| Linear Regression | 0.62 ± 0.14 | 0.57 ± 0.12 | 0.55 ± 0.11 | 0.53 ± 0.10 | 0.49 ± 0.09 |
| Lasso Regression | 0.62 ± 0.14 | 0.56 ± 0.12 | 0.54 ± 0.11 | 0.52 ± 0.11 | 0.49 ± 0.10 |
| RF | 0.77 ± 0.08 | 0.75 ± 0.06 | 0.74 ± 0.06 | 0.74 ± 0.06 | 0.75 ± 0.06 |
| XgBoost | 0.78 ± 0.09 | 0.74 ± 0.06 | 0.72 ± 0.06 | 0.73 ± 0.06 | 0.74 ± 0.06 |
| MLP | 0.73 ± 0.08 | 0.74 ± 0.05 | 0.73 ± 0.05 | 0.72 ± 0.06 | 0.73 ± 0.06 |
| LSTM | 0.72 ± 0.09 | 0.76 ± 0.05 | 0.75 ± 0.05 | 0.75 ± 0.06 | 0.75 ± 0.05 |
| RNN | 0.73 ± 0.09 | 0.73 ± 0.05 | 0.72 ± 0.06 | 0.72 ± 0.06 | 0.72 ± 0.06 |

Shopping mall

| | | | | | |
|---|---|---|---|---|---|
| Linear Regression | 0.68 ± 0.12 | 0.65 ± 0.09 | 0.65 ± 0.08 | 0.63 ± 0.08 | 0.61 ± 0.08 |
| Lasso Regression | 0.68 ± 0.12 | 0.65 ± 0.09 | 0.63 ± 0.08 | 0.63 ± 0.08 | 0.61 ± 0.08 |
| RF | 0.82 ± 0.07 | 0.80 ± 0.05 | 0.79 ± 0.05 | 0.78 ± 0.05 | 0.80 ± 0.05 |
| XgBoost | 0.59 ± 0.07 | 0.58 ± 0.06 | 0.72 ± 0.07 | 0.57 ± 0.06 | 0.57 ± 0.06 |
| MLP | 0.81 ± 0.06 | 0.77 ± 0.06 | 0.77 ± 0.05 | 0.79 ± 0.05 | 0.79 ± 0.05 |
| LSTM | 0.82 ± 0.07 | 0.78 ± 0.05 | 0.78 ± 0.05 | 0.80 ± 0.05 | 0.80 ± 0.05 |
| RNN | 0.77 ± 0.07 | 0.78 ± 0.05 | 0.80 ± 0.04 | 0.78 ± 0.05 | 0.61 ± 0.08 |

The most accurate prediction by considering the mean of all the target features in each time window is shown in bold.

For the shopping mall, all of the non-linear models except the XgBoost model consistently yielded a more accurate average prediction for all of the target features than the linear models in all of the time windows tested (WI>0.77). The LSTM model afforded the most accurate average prediction (WI=0.80-0.82) in three of the five time windows (i.e., real-time prediction, 60-30 and 60-60) and a similar average accuracy to the RF and RNN models in the other two time windows (see Table 4 and FIGS. 10a-10c). The slope and goodness-of-fit of the linear regression of the measured and predicted values further confirmed the predictive accuracy of the LSTM model for all the time windows for the five target features (except for pollen in the 30-15, 60-30 and 60-60 time windows) (see FIGS. 11a1-11a5 and 11d1-11d8). Moreover, the average time required by the LSTM model for training and hyperparameter searching was again reasonably short (˜2.5 h) for the different time windows (See Table 3). It should be noted that in FIGS. 11a1-11d8, the dashed gray line indicates a slope of one.

Permutation importance analysis of input features was conducted using the testing datasets to determine how important each input feature was to the ability of the LSTM model to predict the five target features. A positive feature permutation importance indicates that the feature generates a reduction in predictive accuracy after permutation, while a negative permutation feature importance indicates that the feature has no effect on the accuracy after permutation.³⁶ In the commercial office, RH and the concentrations of PM_{2.5} and PM₁₀ were the three most important features for the accuracy of real-time predictions, while temperature and the concentration of TVOCs were not important features (see Table 5 below). In addition, temperature was consistently among the top 10 most important features for the accuracy of predictions for the 10-5, 60-30 and 60-60 time windows, and the concentration of TVOCs was related to the top 10 most important features for the 30-15 time window. Similar to in the commercial office, in the shopping mall, RH and the concentrations of PM_{2.5} and PM₁₀ were the top three features for real-time predictive accuracy (see Table 6 below). However, unlike in the commercial office, in the shopping mall both RH and temperature were among the top 10 most important features that contributed to the accuracy of predictions for the other time windows.

TABLE 5
Permutation importance of the input features for the LSTM
model for the five time windows in the commercial office.
Real Time Prediction

| Input features¹ | Importance mean | Importance standard deviation |
|---|---|---|
| RH | 3667.29 | 341.17 |
| PM10 | 2058.30 | 854.78 |
| PM2.5 | 1026.11 | 155.84 |
| CO2 | 23.60 | 592.99 |
| TVOC | −250.64 | 480.27 |
| Temperature | −516.90 | 1014.19 |

¹The mean and standard deviation of the permutation feature
importance after five rounds of permutation are shown.

| | | |
|---|---|---|
| Input features^{1,2} | Importance mean | Importance standard deviation |

10-5 Time Window

| | | |
|---|---|---|
| Temperature-4 | 2788.77 | 480.41 |
| Temperature-6 | 2722.59 | 491.50 |
| Temperature-7 | 2598.40 | 440.79 |
| Temperature-9 | 2181.99 | 388.37 |
| Temperature-8 | 2172.46 | 394.11 |
| Temperature-5 | 2152.58 | 374.11 |
| Temperature-2 | 2124.80 | 319.62 |
| Temperature-0 | 1922.18 | 290.67 |
| Temperature-1 | 1741.34 | 288.07 |
| Temperature-3 | 1535.18 | 196.11 |
| PM2.5-3 | 997.86 | 435.69 |
| CO2-9 | 937.40 | 927.92 |
| CO2-3 | 923.51 | 657.31 |
| CO2-8 | 922.69 | 578.29 |
| CO2-7 | 882.38 | 649.59 |
| CO2-2 | 806.67 | 710.39 |
| CO2-5 | 755.74 | 586.83 |
| PM2.5-9 | 755.20 | 278.18 |
| TVOC-8 | 749.75 | 378.39 |
| CO2-6 | 700.73 | 593.15 |
| PM2.5-8 | 676.22 | 212.91 |
| PM2.5-7 | 621.21 | 315.29 |
| TVOC-0 | 616.85 | 516.19 |
| PM2.5-1 | 583.90 | 436.55 |
| PM2.5-4 | 583.35 | 342.52 |
| PM2.5-6 | 570.55 | 320.58 |
| CO2-4 | 561.29 | 642.56 |
| PM2.5-2 | 551.49 | 294.67 |
| CO2-0 | 513.63 | 846.15 |
| TVOC-4 | 491.85 | 217.59 |
| TVOC-2 | 477.41 | 320.45 |
| PM2.5-5 | 473.06 | 301.95 |
| CO2-1 | 466.52 | 863.28 |
| TVOC-9 | 447.18 | 441.73 |
| PM10-3 | 413.96 | 165.38 |
| TVOC-5 | 406.88 | 243.97 |
| TVOC-6 | 405.79 | 316.47 |
| TVOC-3 | 365.21 | 157.61 |
| PM10-9 | 362.49 | 159.04 |
| PM2.5-0 | 343.15 | 155.21 |
| PM10-4 | 338.79 | 218.97 |
| TVOC-1 | 294.13 | 267.96 |
| RH-6 | 294.13 | 256.76 |
| PM10-6 | 279.15 | 138.10 |
| PM10-1 | 263.63 | 260.01 |
| PM10-5 | 261.17 | 121.01 |
| TVOC-7 | 255.18 | 203.73 |
| RH-5 | 235.03 | 188.22 |
| PM10-8 | 220.05 | 163.22 |
| PM10-7 | 200.17 | 160.17 |
| PM10-2 | 195.81 | 243.71 |
| RH-1 | 108.12 | 220.13 |
| RH-2 | −43.57 | 205.53 |
| RH-7 | −74.08 | 188.45 |
| RH-3 | −80.61 | 258.31 |
| RH-4 | −112.75 | 233.60 |
| PM10-0 | −138.35 | 132.48 |
| RH-0 | −254.91 | 378.19 |
| RH-8 | −306.38 | 277.07 |
| RH-9 | −316.73 | 273.89 |

30-15 Time Window

| | | |
|---|---|---|
| TVOC-0 | 687.54 | 200.17 |
| TVOC-4 | 599.13 | 150.78 |
| TVOC-6 | 572.06 | 194.19 |
| TVOC-29 | 567.79 | 94.82 |
| TVOC-2 | 550.59 | 163.31 |
| TVOC-3 | 544.99 | 156.13 |
| TVOC-1 | 491.37 | 182.80 |
| TVOC-7 | 487.38 | 170.47 |
| TVOC-5 | 467.92 | 186.46 |
| TVOC-12 | 440.16 | 68.15 |
| Temperature-27 | 415.97 | 106.65 |
| RH-9 | 408.40 | 12.58 |
| Temperature-25 | 400.13 | 108.28 |
| RH-26 | 399.35 | 80.01 |
| Temperature-18 | 392.88 | 76.98 |
| TVOC-8 | 385.45 | 33.77 |
| Temperature-29 | 384.42 | 97.05 |
| TVOC-28 | 382.42 | 55.00 |
| Temperature-21 | 379.78 | 66.66 |
| TVOC-27 | 379.72 | 64.03 |
| TVOC-26 | 378.32 | 78.90 |
| Temperature-20 | 377.29 | 82.94 |
| Temperature-23 | 376.67 | 94.92 |
| Temperature-9 | 373.43 | 63.80 |
| Temperature-16 | 372.62 | 64.94 |
| Temperature-0 | 348.98 | 71.21 |
| Temperature-22 | 337.16 | 68.18 |
| TVOC-21 | 335.35 | 48.69 |
| Temperature-11 | 334.34 | 68.40 |
| TVOC-15 | 334.00 | 59.55 |
| TVOC-10 | 329.78 | 19.99 |
| RH-10 | 329.65 | 26.10 |
| Temperature-28 | 329.03 | 86.87 |
| Temperature-14 | 327.80 | 54.27 |
| RH-19 | 324.36 | 63.62 |
| RH-12 | 315.56 | 25.49 |
| Temperature-26 | 315.00 | 75.92 |
| RH-17 | 308.08 | 63.86 |
| RH-15 | 307.07 | 50.68 |
| TVOC-11 | 307.04 | 24.14 |
| TVOC-23 | 303.16 | 59.46 |
| Temperature-4 | 302.11 | 54.09 |
| TVOC-13 | 298.05 | 50.59 |
| RH-27 | 286.64 | 75.45 |
| Temperature-17 | 284.14 | 60.60 |
| RH-29 | 283.92 | 91.34 |
| RH-18 | 283.43 | 40.35 |
| RH-0 | 283.40 | 52.90 |
| RH-6 | 282.39 | 44.78 |
| RH-4 | 277.78 | 42.57 |
| TVOC-25 | 272.43 | 75.20 |
| Temperature-5 | 272.19 | 25.16 |
| PM10-18 | 270.71 | 47.95 |
| CO2-0 | 268.23 | 54.01 |
| PM10-24 | 268.21 | 93.07 |
| Temperature-6 | 266.82 | 46.75 |
| Temperature-15 | 261.43 | 36.47 |
| Temperature-13 | 261.09 | 45.80 |
| TVOC-22 | 260.99 | 43.37 |
| RH-20 | 260.40 | 57.16 |
| TVOC-14 | 256.65 | 54.27 |
| Temperature-12 | 255.02 | 50.59 |
| CO2-2 | 254.08 | 53.17 |
| Temperature-2 | 250.29 | 61.25 |
| PM2.5-24 | 248.26 | 62.37 |
| PM10-20 | 245.17 | 48.14 |
| TVOC-24 | 244.46 | 50.26 |
| RH-11 | 243.40 | 29.98 |
| CO2-28 | 243.32 | 105.14 |
| Temperature-3 | 243.10 | 39.89 |
| TVOC-9 | 241.62 | 8.79 |
| TVOC-19 | 239.32 | 73.88 |
| PM10-17 | 239.12 | 41.70 |
| TVOC-18 | 238.61 | 62.75 |
| PM10-25 | 237.55 | 78.85 |
| PM10-19 | 234.02 | 48.40 |
| TVOC-17 | 231.16 | 57.66 |
| RH-14 | 229.81 | 36.62 |
| Temperature-19 | 228.33 | 51.22 |
| PM10-21 | 221.96 | 79.73 |
| Temperature-24 | 220.96 | 69.96 |
| CO2-1 | 220.12 | 47.98 |
| CO2-29 | 217.68 | 118.30 |
| PM10-16 | 217.30 | 72.32 |
| RH-21 | 216.40 | 55.03 |
| RH-13 | 214.95 | 65.17 |
| RH-3 | 211.77 | 84.98 |
| TVOC-20 | 211.31 | 64.01 |
| RH-22 | 209.56 | 73.20 |
| RH-8 | 203.84 | 33.01 |
| RH-7 | 200.28 | 50.38 |
| PM2.5-15 | 198.65 | 101.27 |
| PM2.5-28 | 198.62 | 57.26 |
| RH-5 | 192.91 | 48.83 |
| RH-23 | 191.68 | 81.69 |
| PM2.5-23 | 191.20 | 69.96 |
| PM10-22 | 190.42 | 53.76 |
| Temperature-8 | 188.88 | 66.84 |
| RH-24 | 188.83 | 76.87 |
| PM2.5-18 | 188.08 | 94.34 |
| Temperature-7 | 187.71 | 52.69 |
| Temperature-10 | 187.16 | 59.21 |
| RH-25 | 183.40 | 42.26 |
| RH-28 | 183.24 | 67.74 |
| RH-1 | 183.07 | 59.73 |
| CO2-5 | 182.52 | 46.63 |
| TVOC-16 | 181.97 | 52.07 |
| PM10-28 | 181.82 | 52.95 |
| PM2.5-25 | 181.54 | 53.29 |
| PM10-23 | 180.65 | 58.57 |
| PM10-27 | 178.45 | 64.98 |
| PM2.5-22 | 177.21 | 64.90 |
| PM2.5-16 | 176.23 | 105.57 |
| PM2.5-29 | 175.79 | 64.70 |
| PM2.5-20 | 170.05 | 72.95 |
| PM2.5-27 | 169.73 | 56.94 |
| Temperature-1 | 169.27 | 44.79 |
| CO2-3 | 168.12 | 27.84 |
| PM10-15 | 163.46 | 39.71 |
| PM10-14 | 160.58 | 50.61 |
| PM2.5-8 | 160.27 | 104.40 |
| PM2.5-26 | 152.90 | 69.85 |
| CO2-11 | 152.18 | 34.05 |
| CO2-24 | 148.07 | 95.87 |
| PM10-26 | 146.07 | 48.25 |
| RH-16 | 141.31 | 53.55 |
| RH-2 | 137.80 | 50.49 |
| PM10-13 | 137.02 | 44.36 |
| PM2.5-21 | 136.13 | 41.13 |
| CO2-4 | 134.76 | 30.05 |
| CO2-25 | 122.23 | 111.97 |
| PM2.5-19 | 121.39 | 76.45 |
| PM10-10 | 113.16 | 17.72 |
| PM2.5-9 | 111.37 | 75.91 |
| PM10-8 | 109.42 | 44.70 |
| PM2.5-6 | 106.11 | 78.62 |
| PM2.5-13 | 104.86 | 74.26 |
| PM10-29 | 102.93 | 24.02 |
| PM2.5-17 | 95.85 | 82.11 |
| CO2-27 | 91.33 | 118.10 |
| PM2.5-14 | 89.86 | 68.89 |
| PM10-9 | 89.02 | 17.53 |
| CO2-10 | 88.83 | 25.44 |
| PM2.5-7 | 86.36 | 84.44 |
| PM2.5-10 | 82.96 | 84.26 |
| CO2-6 | 77.87 | 27.57 |
| CO2-16 | 76.08 | 38.60 |
| PM2.5-11 | 74.37 | 55.78 |
| CO2-9 | 66.55 | 34.17 |
| PM10-12 | 60.66 | 36.01 |
| CO2-8 | 60.61 | 28.90 |
| PM10-7 | 56.34 | 57.59 |
| CO2-12 | 56.21 | 29.95 |
| CO2-14 | 46.94 | 36.64 |
| PM2.5-1 | 44.42 | 125.63 |
| PM2.5-2 | 43.17 | 116.54 |
| CO2-20 | 39.73 | 76.09 |
| PM2.5-4 | 36.93 | 82.82 |
| CO2-26 | 32.19 | 89.16 |
| PM2.5-12 | 31.85 | 71.76 |
| CO2-23 | 27.71 | 68.74 |
| CO2-7 | 22.33 | 19.03 |
| PM2.5-3 | 21.74 | 115.09 |
| CO2-21 | 21.58 | 63.84 |
| CO2-13 | 16.32 | 42.94 |
| PM10-0 | 10.94 | 79.22 |
| PM2.5-5 | 7.40 | 84.48 |
| PM10-3 | 6.07 | 24.41 |
| CO2-22 | 0.79 | 73.31 |
| CO2-18 | −1.75 | 36.30 |
| CO2-19 | −2.34 | 54.71 |
| CO2-17 | −4.18 | 46.76 |
| PM10-11 | −5.67 | 24.22 |
| PM10-5 | −22.45 | 71.57 |
| CO2-15 | −25.95 | 30.88 |
| PM10-4 | −31.21 | 60.10 |
| PM2.5-0 | −31.67 | 102.73 |
| PM10-1 | −33.41 | 56.61 |
| PM10-2 | −46.02 | 74.97 |
| PM10-6 | −65.77 | 31.78 |

60-30 Time Window

| | | |
|---|---|---|
| Temperature-42 | 304.57 | 32.02 |
| TVOC-59 | 296.98 | 30.30 |
| Temperature-43 | 294.93 | 24.27 |
| RH-46 | 271.70 | 31.75 |
| Temperature-41 | 267.60 | 28.27 |
| Temperature-29 | 255.02 | 24.41 |
| CO2-0 | 252.43 | 145.75 |
| Temperature-50 | 246.94 | 27.96 |
| Temperature-39 | 241.02 | 34.03 |
| RH-59 | 238.50 | 29.88 |
| RH-56 | 235.51 | 40.14 |
| Temperature-21 | 234.98 | 22.71 |
| Temperature-2 | 234.34 | 37.67 |
| Temperature-40 | 224.13 | 32.79 |
| TVOC-58 | 223.08 | 30.82 |
| Temperature-44 | 222.88 | 22.17 |
| Temperature-16 | 220.08 | 25.95 |
| RH-44 | 215.46 | 27.12 |
| Temperature-4 | 215.04 | 32.03 |
| Temperature-48 | 213.90 | 20.42 |
| Temperature-56 | 213.86 | 19.05 |
| Temperature-59 | 213.46 | 22.53 |
| RH-58 | 213.07 | 29.12 |
| Temperature-32 | 212.67 | 18.94 |
| CO2-4 | 212.36 | 117.02 |
| Temperature-52 | 211.39 | 25.50 |
| Temperature-22 | 211.16 | 30.62 |
| Temperature-51 | 209.34 | 17.83 |
| RH-55 | 206.62 | 17.75 |
| CO2-9 | 206.00 | 68.20 |
| Temperature-38 | 205.43 | 20.99 |
| RH-54 | 203.58 | 31.23 |
| CO2-3 | 201.77 | 118.76 |
| Temperature-35 | 201.73 | 35.96 |
| Temperature-47 | 198.73 | 18.80 |
| TVOC-43 | 198.59 | 40.83 |
| Temperature-1 | 196.73 | 29.23 |
| CO2-1 | 196.56 | 124.25 |
| CO2-7 | 195.53 | 92.50 |
| CO2-2 | 195.26 | 121.54 |
| RH-57 | 193.91 | 44.11 |
| Temperature-58 | 191.90 | 16.94 |
| RH-48 | 190.56 | 31.10 |
| TVOC-37 | 189.98 | 31.78 |
| TVOC-4 | 189.46 | 82.65 |
| Temperature-46 | 187.29 | 28.06 |
| Temperature-36 | 185.59 | 27.53 |
| Temperature-34 | 185.16 | 20.98 |
| Temperature-55 | 183.71 | 14.01 |
| CO2-8 | 182.56 | 91.57 |
| Temperature-17 | 181.15 | 25.75 |
| TVOC-47 | 180.62 | 26.67 |
| TVOC-34 | 180.38 | 46.34 |
| Temperature-0 | 179.89 | 30.31 |
| Temperature-33 | 175.56 | 20.03 |
| CO2-11 | 173.90 | 69.66 |
| CO2-5 | 173.83 | 105.90 |
| RH-49 | 172.45 | 30.73 |
| CO2-6 | 172.35 | 89.91 |
| Temperature-7 | 172.35 | 25.56 |
| Temperature-27 | 171.68 | 21.29 |
| TVOC-22 | 171.35 | 28.10 |
| RH-33 | 171.21 | 45.23 |
| RH-50 | 168.88 | 41.44 |
| RH-52 | 166.44 | 39.37 |
| CO2-10 | 166.32 | 85.44 |
| RH-28 | 166.20 | 31.64 |
| Temperature-9 | 163.92 | 20.88 |
| RH-51 | 163.74 | 38.41 |
| Temperature-5 | 163.36 | 23.36 |
| RH-35 | 162.56 | 38.23 |
| Temperature-6 | 161.67 | 25.37 |
| Temperature-15 | 160.98 | 29.01 |
| TVOC-30 | 160.03 | 25.74 |
| Temperature-53 | 156.19 | 10.28 |
| Temperature-19 | 156.19 | 21.72 |
| Temperature-30 | 155.67 | 21.78 |
| Temperature-20 | 154.54 | 20.35 |
| TVOC-32 | 152.82 | 17.48 |
| Temperature-37 | 152.56 | 25.28 |
| TVOC-39 | 152.48 | 38.21 |
| Temperature-13 | 150.86 | 25.59 |
| Temperature-8 | 147.46 | 24.87 |
| TVOC-31 | 146.94 | 22.40 |
| RH-34 | 144.15 | 45.40 |
| Temperature-26 | 144.09 | 31.40 |
| CO2-12 | 144.03 | 70.44 |
| Temperature-49 | 142.78 | 14.48 |
| Temperature-45 | 142.55 | 30.71 |
| Temperature-24 | 142.50 | 18.62 |
| Temperature-28 | 142.23 | 18.77 |
| Temperature-54 | 142.22 | 27.91 |
| Temperature-57 | 141.68 | 29.17 |
| TVOC-35 | 141.51 | 33.05 |
| TVOC-0 | 140.98 | 40.88 |
| RH-47 | 140.25 | 35.79 |
| Temperature-31 | 138.43 | 14.16 |
| CO2-16 | 137.95 | 62.41 |
| TVOC-57 | 137.65 | 24.50 |
| RH-45 | 137.42 | 25.07 |
| TVOC-45 | 137.34 | 21.26 |
| TVOC-33 | 136.87 | 47.93 |
| TVOC-29 | 136.59 | 19.16 |
| PM2.5-59 | 136.49 | 17.40 |
| TVOC-51 | 134.34 | 24.93 |
| Temperature-25 | 134.01 | 19.34 |
| RH-19 | 131.07 | 30.86 |
| TVOC-3 | 130.89 | 54.92 |
| RH-16 | 128.68 | 23.23 |
| Temperature-3 | 128.02 | 33.51 |
| RH-38 | 127.84 | 42.11 |
| Temperature-14 | 127.12 | 22.65 |
| TVOC-50 | 126.25 | 12.97 |
| RH-30 | 125.36 | 41.78 |
| TVOC-27 | 124.63 | 31.80 |
| TVOC-28 | 123.30 | 24.38 |
| TVOC-42 | 123.03 | 19.29 |
| RH-53 | 117.97 | 35.06 |
| TVOC-46 | 115.24 | 12.41 |
| RH-41 | 113.38 | 37.08 |
| TVOC-44 | 113.36 | 19.59 |
| CO2-14 | 112.90 | 55.09 |
| Temperature-18 | 111.82 | 17.96 |
| RH-23 | 109.89 | 29.90 |
| TVOC-23 | 109.42 | 45.10 |
| RH-29 | 109.41 | 27.97 |
| TVOC-41 | 108.04 | 25.08 |
| RH-36 | 107.07 | 28.33 |
| TVOC-52 | 105.60 | 25.89 |
| Temperature-23 | 104.91 | 9.96 |
| TVOC-10 | 104.86 | 28.92 |
| TVOC-48 | 103.16 | 10.32 |
| RH-43 | 100.50 | 36.63 |
| TVOC-26 | 100.23 | 24.79 |
| TVOC-8 | 98.77 | 35.22 |
| Temperature-11 | 98.04 | 16.28 |
| PM2.5-57 | 96.78 | 13.48 |
| PM2.5-58 | 94.07 | 9.28 |
| TVOC-1 | 93.20 | 54.78 |
| TVOC-40 | 93.07 | 29.98 |
| TVOC-55 | 92.44 | 13.49 |
| TVOC-38 | 91.46 | 28.14 |
| Temperature-12 | 90.79 | 21.37 |
| TVOC-24 | 89.81 | 27.54 |
| RH-37 | 88.80 | 42.09 |
| TVOC-36 | 87.25 | 41.02 |
| TVOC-49 | 86.75 | 10.84 |
| CO2-20 | 85.18 | 40.21 |
| CO2-21 | 84.10 | 50.57 |
| CO2-13 | 83.76 | 54.29 |
| RH-10 | 77.37 | 32.32 |
| TVOC-6 | 76.05 | 23.97 |
| TVOC-19 | 74.47 | 28.68 |
| RH-2 | 72.22 | 15.81 |
| RH-25 | 67.91 | 30.01 |
| RH-26 | 67.60 | 42.76 |
| TVOC-9 | 67.52 | 39.36 |
| RH-21 | 66.98 | 26.09 |
| Temperature-10 | 65.91 | 25.42 |
| RH-9 | 65.88 | 22.73 |
| TVOC-53 | 64.60 | 22.08 |
| RH-7 | 64.36 | 10.76 |
| RH-1 | 63.63 | 14.80 |
| CO2-18 | 61.19 | 59.87 |
| RH-31 | 60.79 | 47.96 |
| TVOC-5 | 60.57 | 12.83 |
| RH-12 | 60.57 | 25.30 |
| PM2.5-56 | 60.42 | 17.83 |
| RH-11 | 59.18 | 18.97 |
| PM2.5-2 | 57.43 | 23.63 |
| CO2-15 | 56.88 | 49.50 |
| PM2.5-54 | 56.56 | 14.15 |
| RH-42 | 56.30 | 32.23 |
| RH-20 | 55.65 | 19.44 |
| TVOC-56 | 55.62 | 25.93 |
| TVOC-25 | 55.05 | 25.49 |
| TVOC-7 | 51.49 | 9.00 |
| PM10-18 | 50.95 | 18.90 |
| RH-39 | 50.21 | 40.20 |
| RH-17 | 47.05 | 21.33 |
| PM2.5-8 | 46.84 | 10.41 |
| PM2.5-32 | 46.49 | 13.35 |
| CO2-19 | 46.13 | 48.66 |
| RH-24 | 45.32 | 37.46 |
| PM2.5-6 | 45.22 | 29.68 |
| RH-4 | 45.00 | 10.60 |
| TVOC-54 | 44.33 | 20.22 |
| CO2-25 | 42.86 | 26.77 |
| PM2.5-19 | 42.65 | 18.47 |
| PM2.5-48 | 42.35 | 13.91 |
| PM10-27 | 41.36 | 24.87 |
| RH-13 | 41.06 | 21.01 |
| PM2.5-49 | 41.03 | 11.82 |
| PM10-58 | 40.80 | 19.64 |
| TVOC-2 | 38.37 | 25.15 |
| TVOC-18 | 36.51 | 32.41 |
| PM2.5-55 | 35.93 | 21.61 |
| CO2-22 | 35.87 | 26.16 |
| PM10-51 | 35.23 | 19.67 |
| PM10-1 | 35.18 | 22.31 |
| PM2.5-17 | 34.84 | 19.80 |
| PM2.5-7 | 34.77 | 14.07 |
| PM10-17 | 34.04 | 15.63 |
| CO2-17 | 33.21 | 48.16 |
| RH-32 | 32.18 | 34.54 |
| RH-22 | 31.89 | 33.03 |
| CO2-26 | 29.63 | 33.66 |
| RH-27 | 29.13 | 51.29 |
| PM10-20 | 27.37 | 27.04 |
| PM2.5-51 | 27.25 | 10.79 |
| PM10-52 | 26.05 | 15.43 |
| RH-8 | 25.93 | 31.12 |
| PM2.5-53 | 25.30 | 18.09 |
| CO2-23 | 25.15 | 33.97 |
| PM2.5-4 | 23.62 | 20.53 |
| PM2.5-52 | 23.11 | 13.01 |
| PM2.5-18 | 23.05 | 17.75 |
| PM2.5-23 | 23.05 | 18.50 |
| PM2.5-20 | 22.81 | 15.23 |
| CO2-24 | 22.25 | 29.38 |
| CO2-27 | 20.17 | 34.78 |
| TVOC-21 | 19.91 | 15.99 |
| PM10-35 | 18.63 | 8.45 |
| CO2-29 | 17.94 | 28.71 |
| PM2.5-21 | 16.88 | 16.05 |
| PM2.5-37 | 16.51 | 9.85 |
| RH-3 | 15.79 | 7.85 |
| TVOC-15 | 15.68 | 36.03 |
| PM2.5-22 | 15.63 | 15.36 |
| PM10-10 | 14.89 | 15.97 |
| PM2.5-3 | 14.41 | 16.73 |
| PM10-4 | 14.05 | 12.65 |
| TVOC-11 | 14.01 | 17.69 |
| PM2.5-5 | 13.57 | 23.60 |
| PM2.5-42 | 13.25 | 18.33 |
| PM2.5-24 | 12.80 | 19.47 |
| PM10-26 | 12.38 | 27.10 |
| PM2.5-40 | 12.34 | 14.73 |
| PM10-30 | 12.31 | 9.68 |
| PM10-59 | 12.08 | 16.82 |
| PM10-48 | 11.79 | 12.83 |
| RH-40 | 10.76 | 44.18 |
| CO2-33 | 9.59 | 33.46 |
| PM10-8 | 9.23 | 12.23 |
| CO2-31 | 8.73 | 30.86 |
| PM2.5-26 | 6.72 | 21.44 |
| PM10-49 | 6.23 | 14.04 |
| TVOC-12 | 5.70 | 35.60 |
| RH-18 | 5.08 | 24.86 |
| PM2.5-38 | 4.92 | 14.53 |
| RH-0 | 4.61 | 15.63 |
| TVOC-14 | 4.52 | 24.44 |
| PM10-9 | 3.94 | 11.49 |
| PM10-3 | 3.29 | 22.00 |
| PM10-12 | 3.24 | 16.79 |
| PM2.5-30 | 3.17 | 14.03 |
| PM10-0 | 2.61 | 26.83 |
| PM2.5-33 | 2.19 | 9.98 |
| PM2.5-43 | 1.92 | 22.62 |
| CO2-40 | 1.30 | 29.53 |
| RH-15 | −1.12 | 20.05 |
| PM2.5-10 | −1.14 | 8.38 |
| TVOC-17 | −1.25 | 23.04 |
| PM2.5-50 | −1.38 | 24.99 |
| PM10-6 | −1.73 | 12.93 |
| RH-5 | −2.73 | 23.92 |
| PM2.5-34 | −3.19 | 3.85 |
| PM2.5-1 | −3.47 | 27.20 |
| PM2.5-11 | −4.00 | 5.19 |
| PM10-28 | −4.03 | 14.88 |
| PM10-2 | −4.24 | 28.78 |
| PM2.5-45 | −4.48 | 16.52 |
| PM10-57 | −4.61 | 24.76 |
| PM10-50 | −5.01 | 19.08 |
| PM2.5-9 | −5.05 | 7.17 |
| TVOC-20 | −5.36 | 35.03 |
| PM10-32 | −5.54 | 15.23 |
| CO2-30 | −5.59 | 23.87 |
| CO2-28 | −5.74 | 29.10 |
| PM2.5-27 | −6.04 | 19.14 |
| PM10-39 | −8.05 | 23.81 |
| PM10-19 | −8.67 | 12.84 |
| PM2.5-35 | −9.63 | 10.95 |
| PM10-11 | −10.02 | 15.81 |
| PM10-47 | −10.45 | 18.15 |
| PM2.5-36 | −10.88 | 3.99 |
| PM10-54 | −11.18 | 22.52 |
| TVOC-13 | −11.27 | 23.48 |
| PM10-53 | −11.41 | 24.25 |
| PM2.5-41 | −11.99 | 5.94 |
| PM10-25 | −13.05 | 28.80 |
| PM2.5-31 | −13.20 | 10.06 |
| PM10-45 | −17.42 | 28.19 |
| PM10-24 | −17.84 | 28.11 |
| PM10-14 | −18.41 | 16.44 |
| PM2.5-0 | −18.65 | 25.30 |
| PM10-41 | −19.01 | 24.43 |
| PM10-22 | −19.21 | 21.31 |
| RH-14 | −19.37 | 23.70 |
| PM10-5 | −21.22 | 13.50 |
| PM10-16 | −21.48 | 13.79 |
| PM10-15 | −21.92 | 9.49 |
| PM10-42 | −22.00 | 28.90 |
| PM10-29 | −22.18 | 6.63 |
| PM2.5-25 | −22.33 | 20.33 |
| PM2.5-39 | −24.72 | 12.65 |
| PM2.5-29 | −25.28 | 27.68 |
| PM10-56 | −25.67 | 18.29 |
| PM2.5-16 | −25.86 | 18.83 |
| PM2.5-28 | −25.96 | 16.23 |
| PM10-44 | −26.04 | 26.09 |
| PM2.5-15 | −26.06 | 19.75 |
| PM2.5-47 | −29.41 | 15.45 |
| PM10-36 | −29.81 | 13.04 |
| PM10-31 | −30.99 | 13.01 |
| CO2-37 | −32.63 | 28.35 |
| PM2.5-14 | −34.76 | 21.96 |
| CO2-32 | −35.87 | 28.03 |
| PM2.5-12 | −38.94 | 5.74 |
| PM10-46 | −38.97 | 18.08 |
| PM10-13 | −39.64 | 8.60 |
| PM2.5-46 | −40.25 | 17.04 |
| PM10-55 | −40.31 | 28.40 |
| PM2.5-44 | −41.94 | 28.31 |
| PM10-40 | −43.16 | 22.78 |
| CO2-35 | −43.54 | 37.38 |
| PM10-21 | −43.61 | 18.49 |
| RH-6 | −44.84 | 27.52 |
| PM10-37 | −45.04 | 18.66 |
| PM10-38 | −45.24 | 23.39 |
| PM10-34 | −45.84 | 17.71 |
| PM10-23 | −47.58 | 30.88 |
| CO2-42 | −48.27 | 39.13 |
| PM10-43 | −48.54 | 28.91 |
| CO2-52 | −50.02 | 36.63 |
| CO2-59 | −50.82 | 65.89 |
| PM10-33 | −53.91 | 27.55 |
| CO2-36 | −54.02 | 36.62 |
| PM2.5-13 | −55.14 | 15.73 |
| PM10-7 | −57.91 | 13.13 |
| CO2-38 | −63.32 | 29.60 |
| TVOC-16 | −66.54 | 22.92 |
| CO2-39 | −66.95 | 20.34 |
| CO2-34 | −68.89 | 24.99 |
| CO2-41 | −72.63 | 27.99 |
| CO2-56 | −73.20 | 51.01 |
| CO2-44 | −88.50 | 21.92 |
| CO2-47 | −91.41 | 40.59 |
| CO2-58 | −91.47 | 61.78 |
| CO2-57 | −98.52 | 60.23 |
| CO2-53 | −101.95 | 47.60 |
| CO2-45 | −108.60 | 42.34 |
| CO2-51 | −111.39 | 41.40 |
| CO2-49 | −113.02 | 35.89 |
| CO2-46 | −113.85 | 22.92 |
| CO2-55 | −114.02 | 37.90 |
| CO2-50 | −117.92 | 39.67 |
| CO2-54 | −118.80 | 54.47 |
| CO2-48 | −132.92 | 37.13 |
| CO2-43 | −153.54 | 36.60 |

60-60 Time Window

| | | |
|---|---|---|
| Temperature-37 | 479.72 | 42.02 |
| Temperature-52 | 468.51 | 43.52 |
| Temperature-47 | 465.78 | 43.02 |
| Temperature-45 | 449.86 | 46.42 |
| Temperature-48 | 446.52 | 37.41 |
| Temperature-31 | 436.45 | 35.03 |
| Temperature-46 | 434.06 | 37.96 |
| Temperature-1 | 424.60 | 39.05 |
| Temperature-57 | 423.50 | 29.66 |
| Temperature-54 | 399.53 | 34.49 |
| Temperature-20 | 393.61 | 31.52 |
| Temperature-21 | 393.15 | 31.38 |
| Temperature-41 | 388.10 | 40.86 |
| Temperature-50 | 385.33 | 36.66 |
| Temperature-28 | 384.34 | 22.12 |
| Temperature-59 | 379.40 | 27.96 |
| Temperature-29 | 377.92 | 32.95 |
| Temperature-42 | 377.39 | 31.74 |
| Temperature-24 | 375.45 | 35.99 |
| Temperature-25 | 370.36 | 34.05 |
| Temperature-35 | 366.83 | 36.74 |
| Temperature-43 | 364.89 | 30.19 |
| Temperature-30 | 360.07 | 29.80 |
| Temperature-4 | 357.41 | 28.38 |
| Temperature-40 | 353.04 | 33.71 |
| Temperature-49 | 352.36 | 31.60 |
| Temperature-32 | 352.21 | 28.11 |
| Temperature-15 | 347.42 | 38.23 |
| Temperature-53 | 345.98 | 22.70 |
| Temperature-16 | 340.89 | 27.43 |
| Temperature-10 | 338.80 | 27.72 |
| CO2-1 | 335.72 | 35.72 |
| Temperature-13 | 335.27 | 19.12 |
| Temperature-38 | 335.19 | 28.68 |
| Temperature-36 | 332.76 | 26.20 |
| Temperature-8 | 323.57 | 21.11 |
| Temperature-33 | 322.32 | 31.64 |
| Temperature-58 | 321.22 | 36.65 |
| CO2-0 | 319.09 | 41.00 |
| Temperature-6 | 308.00 | 28.24 |
| Temperature-9 | 306.48 | 21.70 |
| Temperature-27 | 306.29 | 27.11 |
| Temperature-39 | 301.73 | 25.94 |
| Temperature-18 | 299.60 | 26.36 |
| Temperature-19 | 294.48 | 22.58 |
| CO2-2 | 290.64 | 32.92 |
| Temperature-22 | 290.30 | 27.01 |
| Temperature-34 | 288.63 | 33.23 |
| Temperature-7 | 287.49 | 29.25 |
| Temperature-44 | 287.45 | 29.89 |
| Temperature-26 | 285.40 | 27.97 |
| Temperature-5 | 281.79 | 23.25 |
| Temperature-0 | 279.13 | 29.99 |
| Temperature-55 | 275.14 | 32.93 |
| CO2-4 | 265.50 | 19.42 |
| Temperature-23 | 263.10 | 22.30 |
| Temperature-2 | 260.90 | 22.11 |
| Temperature-11 | 260.56 | 24.13 |
| Temperature-3 | 256.80 | 30.43 |
| PM2.5-59 | 256.19 | 79.66 |
| Temperature-56 | 254.86 | 30.42 |
| Temperature-17 | 254.40 | 17.08 |
| CO2-3 | 252.35 | 31.45 |
| Temperature-14 | 251.29 | 22.79 |
| RH-49 | 244.15 | 28.62 |
| Temperature-12 | 241.87 | 18.60 |
| TVOC-44 | 231.88 | 81.99 |
| Temperature-51 | 216.27 | 24.65 |
| PM2.5-58 | 214.37 | 69.73 |
| TVOC-52 | 214.14 | 125.74 |
| PM2.5-57 | 199.71 | 63.14 |
| PM2.5-35 | 199.33 | 52.91 |
| PM2.5-34 | 194.05 | 53.24 |
| CO2-5 | 190.52 | 13.65 |
| PM2.5-56 | 182.69 | 66.82 |
| TVOC-0 | 181.14 | 24.72 |
| TVOC-51 | 173.88 | 123.88 |
| CO2-9 | 171.53 | 14.77 |
| TVOC-34 | 171.30 | 31.46 |
| RH-35 | 170.20 | 28.47 |
| CO2-7 | 169.78 | 22.60 |
| TVOC-41 | 163.93 | 45.05 |
| RH-59 | 163.93 | 25.29 |
| PM2.5-47 | 163.82 | 54.62 |
| PM2.5-45 | 163.25 | 53.16 |
| TVOC-27 | 162.83 | 44.65 |
| RH-19 | 157.21 | 28.64 |
| PM2.5-37 | 157.17 | 42.60 |
| PM2.5-36 | 156.94 | 56.44 |
| CO2-11 | 156.83 | 18.29 |
| PM2.5-41 | 154.21 | 42.10 |
| TVOC-47 | 153.60 | 42.83 |
| PM2.5-42 | 151.89 | 38.93 |
| RH-47 | 151.44 | 32.98 |
| PM2.5-50 | 148.78 | 52.18 |
| PM2.5-33 | 148.36 | 41.24 |
| TVOC-1 | 146.46 | 16.96 |
| RH-46 | 142.96 | 30.28 |
| TVOC-46 | 141.83 | 56.88 |
| TVOC-53 | 141.45 | 95.63 |
| TVOC-45 | 140.04 | 31.84 |
| CO2-6 | 138.71 | 15.99 |
| PM2.5-38 | 138.56 | 41.15 |
| TVOC-4 | 135.29 | 22.94 |
| TVOC-54 | 133.81 | 106.75 |
| TVOC-31 | 132.86 | 26.43 |
| PM2.5-49 | 131.99 | 48.73 |
| TVOC-28 | 131.53 | 28.32 |
| PM2.5-46 | 129.71 | 37.11 |
| PM2.5-54 | 129.52 | 37.98 |
| PM2.5-32 | 128.30 | 38.50 |
| PM10-52 | 127.70 | 60.93 |
| RH-2 | 126.18 | 41.25 |
| RH-8 | 125.04 | 31.50 |
| RH-39 | 124.35 | 40.48 |
| TVOC-26 | 123.59 | 24.20 |
| RH-17 | 123.33 | 22.18 |
| TVOC-35 | 123.06 | 22.67 |
| TVOC-42 | 122.83 | 40.07 |
| TVOC-29 | 121.81 | 23.05 |
| TVOC-33 | 121.51 | 21.17 |
| RH-21 | 120.52 | 18.66 |
| RH-22 | 120.10 | 18.94 |
| RH-25 | 118.81 | 21.21 |
| PM10-55 | 114.55 | 27.92 |
| CO2-8 | 112.43 | 26.45 |
| RH-14 | 112.24 | 26.97 |
| TVOC-43 | 111.93 | 33.53 |
| TVOC-30 | 111.36 | 25.41 |
| TVOC-49 | 109.69 | 48.08 |
| PM2.5-52 | 108.93 | 49.90 |
| RH-57 | 107.79 | 29.63 |
| PM2.5-48 | 107.00 | 58.96 |
| PM2.5-43 | 104.68 | 36.01 |
| PM10-57 | 104.03 | 48.86 |
| PM2.5-31 | 103.81 | 48.89 |
| PM2.5-55 | 103.43 | 35.48 |
| TVOC-39 | 102.89 | 34.23 |
| TVOC-38 | 101.98 | 35.51 |
| CO2-10 | 100.20 | 25.67 |
| PM10-53 | 99.97 | 54.87 |
| PM2.5-30 | 99.51 | 49.90 |
| RH-29 | 98.91 | 34.62 |
| PM10-54 | 98.87 | 47.45 |
| PM10-59 | 97.42 | 42.88 |
| PM10-51 | 90.82 | 39.03 |
| RH-58 | 90.51 | 26.45 |
| RH-34 | 89.98 | 28.62 |
| TVOC-5 | 89.22 | 16.80 |
| TVOC-40 | 88.31 | 46.46 |
| PM10-28 | 87.25 | 42.16 |
| PM10-50 | 86.64 | 38.98 |
| RH-15 | 85.57 | 22.73 |
| TVOC-3 | 85.23 | 16.71 |
| TVOC-50 | 84.59 | 42.86 |
| RH-51 | 84.43 | 29.23 |
| TVOC-11 | 84.28 | 25.45 |
| RH-43 | 82.88 | 32.58 |
| PM2.5-39 | 82.57 | 27.82 |
| PM10-58 | 82.35 | 36.00 |
| PM10-0 | 81.47 | 34.16 |
| PM2.5-53 | 79.19 | 47.20 |
| TVOC-12 | 79.19 | 24.59 |
| TVOC-58 | 78.66 | 26.14 |
| RH-36 | 77.22 | 26.12 |
| TVOC-48 | 75.66 | 40.94 |
| RH-26 | 74.56 | 21.81 |
| TVOC-57 | 72.77 | 51.75 |
| RH-18 | 72.13 | 23.28 |
| RH-20 | 70.84 | 23.46 |
| PM2.5-51 | 70.53 | 37.64 |
| TVOC-25 | 69.55 | 18.97 |
| CO2-21 | 69.28 | 27.37 |
| TVOC-32 | 68.98 | 15.04 |
| RH-1 | 68.67 | 38.94 |
| PM2.5-40 | 67.15 | 34.81 |
| RH-23 | 67.00 | 19.73 |
| CO2-12 | 66.85 | 24.79 |
| PM10-49 | 66.13 | 51.89 |
| TVOC-2 | 65.60 | 17.16 |
| TVOC-36 | 65.37 | 20.66 |
| TVOC-8 | 64.80 | 14.64 |
| PM2.5-29 | 64.04 | 34.44 |
| RH-16 | 63.70 | 13.58 |
| CO2-17 | 63.24 | 19.19 |
| PM10-1 | 62.59 | 51.80 |
| CO2-14 | 62.56 | 23.79 |
| PM2.5-44 | 60.92 | 31.99 |
| PM10-41 | 60.62 | 22.62 |
| RH-37 | 59.78 | 25.40 |
| RH-3 | 59.67 | 32.78 |
| PM10-40 | 57.81 | 26.87 |
| RH-52 | 57.62 | 38.85 |
| CO2-18 | 57.16 | 20.25 |
| CO2-16 | 56.90 | 16.21 |
| PM10-48 | 55.34 | 38.64 |
| PM10-56 | 55.11 | 33.92 |
| PM10-5 | 54.88 | 38.88 |
| RH-6 | 54.05 | 34.99 |
| TVOC-59 | 53.52 | 3.00 |
| PM10-7 | 53.14 | 27.84 |
| RH-42 | 52.72 | 34.45 |
| TVOC-37 | 51.81 | 18.20 |
| PM10-17 | 50.93 | 29.95 |
| TVOC-10 | 50.93 | 13.52 |
| TVOC-56 | 49.30 | 61.91 |
| RH-13 | 47.52 | 30.07 |
| TVOC-17 | 47.21 | 22.25 |
| PM2.5-1 | 46.91 | 56.72 |
| RH-41 | 46.41 | 33.77 |
| PM10-30 | 46.26 | 37.45 |
| PM2.5-3 | 46.00 | 42.64 |
| PM10-18 | 45.88 | 29.67 |
| CO2-13 | 44.89 | 28.05 |
| CO2-24 | 43.00 | 33.34 |
| TVOC-7 | 42.77 | 22.44 |
| RH-0 | 41.59 | 35.05 |
| PM10-25 | 38.82 | 34.75 |
| RH-45 | 38.70 | 32.81 |
| PM10-27 | 38.17 | 20.23 |
| PM10-37 | 36.88 | 33.07 |
| PM2.5-6 | 36.61 | 41.77 |
| PM10-29 | 35.25 | 38.87 |
| RH-48 | 34.91 | 42.00 |
| PM10-16 | 34.91 | 31.07 |
| TVOC-55 | 34.87 | 77.61 |
| PM10-38 | 34.18 | 26.39 |
| PM10-20 | 33.16 | 27.27 |
| PM10-26 | 33.08 | 28.69 |
| PM2.5-2 | 32.70 | 31.53 |
| PM10-39 | 32.66 | 26.79 |
| RH-50 | 32.25 | 30.44 |
| RH-56 | 30.42 | 28.12 |
| RH-11 | 29.85 | 28.51 |
| PM2.5-26 | 29.17 | 43.33 |
| CO2-20 | 29.13 | 17.28 |
| RH-33 | 24.08 | 23.99 |
| PM10-22 | 23.25 | 35.63 |
| CO2-15 | 20.28 | 19.26 |
| PM10-14 | 19.37 | 21.79 |
| PM10-31 | 19.11 | 45.73 |
| PM10-44 | 19.03 | 19.96 |
| PM10-4 | 18.31 | 39.42 |
| CO2-26 | 15.76 | 29.26 |
| PM2.5-23 | 15.76 | 42.36 |
| PM10-36 | 15.72 | 33.83 |
| PM10-35 | 14.02 | 38.12 |
| PM10-2 | 13.67 | 32.85 |
| PM10-34 | 13.14 | 34.41 |
| CO2-19 | 13.10 | 23.01 |
| PM2.5-7 | 12.84 | 33.63 |
| RH-53 | 12.84 | 36.68 |
| PM2.5-28 | 12.34 | 36.34 |
| PM10-8 | 12.23 | 32.27 |
| CO2-22 | 9.88 | 32.31 |
| TVOC-22 | 8.74 | 11.05 |
| PM10-33 | 8.62 | 32.42 |
| TVOC-14 | 7.67 | 9.93 |
| RH-40 | 6.91 | 39.69 |
| PM10-46 | 6.12 | 24.42 |
| PM10-9 | 6.12 | 26.72 |
| PM10-32 | 5.62 | 40.03 |
| RH-9 | 5.20 | 25.77 |
| PM10-45 | 4.52 | 20.85 |
| TVOC-24 | 3.72 | 11.56 |
| PM10-19 | 3.65 | 27.75 |
| RH-44 | 3.38 | 41.10 |
| PM10-15 | 2.77 | 33.74 |
| PM10-42 | 2.66 | 9.96 |
| TVOC-9 | 2.54 | 12.55 |
| RH-10 | 1.41 | 36.94 |
| RH-12 | 0.15 | 21.06 |
| TVOC-15 | −6.42 | 17.87 |
| PM10-3 | −6.49 | 32.97 |
| PM2.5-4 | −6.84 | 34.07 |
| RH-31 | −7.37 | 33.08 |
| PM10-47 | −7.94 | 19.41 |
| PM10-6 | −8.66 | 33.11 |
| TVOC-6 | −9.00 | 4.63 |
| PM2.5-5 | −10.03 | 37.40 |
| PM2.5-21 | −10.18 | 34.80 |
| CO2-23 | −11.55 | 26.82 |
| PM2.5-10 | −11.96 | 24.80 |
| PM10-23 | −12.15 | 24.58 |
| PM2.5-27 | −13.29 | 31.88 |
| PM10-10 | −13.94 | 29.94 |
| RH-7 | −15.04 | 29.31 |
| PM2.5-25 | −16.37 | 34.97 |
| PM2.5-22 | −16.52 | 25.07 |
| RH-28 | −18.04 | 25.91 |
| PM10-24 | −18.42 | 32.21 |
| CO2-25 | −20.43 | 24.07 |
| PM10-43 | −20.59 | 12.14 |
| TVOC-13 | −21.54 | 6.95 |
| RH-30 | −22.07 | 26.09 |
| RH-32 | −22.45 | 24.06 |
| TVOC-23 | −24.08 | 3.23 |
| RH-55 | −24.65 | 33.37 |
| PM10-21 | −25.14 | 20.91 |
| RH-54 | −26.02 | 27.66 |
| RH-5 | −28.49 | 44.72 |
| PM10-12 | −28.75 | 27.95 |
| PM2.5-24 | −30.96 | 35.47 |
| RH-4 | −32.21 | 31.21 |
| TVOC-19 | −32.70 | 13.08 |
| RH-27 | −33.04 | 36.15 |
| RH-24 | −34.56 | 14.19 |
| RH-38 | −34.79 | 35.72 |
| PM10-13 | −36.92 | 29.51 |
| PM2.5-8 | −39.58 | 32.35 |
| PM2.5-11 | −40.15 | 27.01 |
| PM2.5-9 | −41.51 | 35.09 |
| PM10-11 | −45.27 | 29.87 |
| CO2-28 | −53.02 | 35.58 |
| PM2.5-19 | −54.69 | 25.85 |
| PM2.5-0 | −55.68 | 26.54 |
| PM2.5-12 | −69.17 | 24.51 |
| CO2-27 | −71.18 | 38.98 |
| PM2.5-15 | −71.67 | 19.89 |
| TVOC-16 | −72.20 | 8.74 |
| PM2.5-18 | −73.76 | 26.13 |
| CO2-31 | −77.75 | 38.22 |
| TVOC-21 | −78.21 | 20.18 |
| CO2-30 | −82.69 | 37.33 |
| PM2.5-20 | −84.62 | 31.08 |
| PM2.5-13 | −87.74 | 20.94 |
| CO2-39 | −90.74 | 43.21 |
| CO2-56 | −95.72 | 33.17 |
| CO2-29 | −96.28 | 38.11 |
| TVOC-18 | −99.97 | 21.88 |
| CO2-44 | −106.92 | 37.12 |
| CO2-33 | −107.38 | 28.68 |
| TVOC-20 | −110.00 | 15.75 |
| CO2-48 | −111.90 | 45.87 |
| CO2-42 | −112.47 | 37.00 |
| CO2-38 | −114.63 | 36.90 |
| PM2.5-17 | −117.06 | 21.20 |
| PM2.5-14 | −118.66 | 25.21 |
| CO2-53 | −119.99 | 32.29 |
| CO2-58 | −121.77 | 40.05 |
| CO2-54 | −123.71 | 37.44 |
| CO2-32 | −125.04 | 43.60 |
| CO2-43 | −127.09 | 30.76 |
| PM2.5-16 | −128.84 | 26.09 |
| CO2-50 | −130.35 | 37.21 |
| CO2-49 | −132.33 | 37.54 |
| CO2-36 | −134.49 | 40.47 |
| CO2-41 | −135.03 | 42.48 |
| CO2-34 | −137.76 | 39.70 |
| CO2-35 | −139.17 | 34.57 |
| CO2-40 | −141.64 | 31.42 |
| CO2-47 | −141.67 | 32.64 |
| CO2-46 | −147.67 | 45.52 |
| CO2-51 | −149.31 | 47.88 |
| CO2-37 | −151.78 | 36.07 |
| CO2-55 | −156.26 | 37.45 |
| CO2-57 | −170.96 | 58.43 |
| CO2-45 | −178.90 | 34.14 |
| CO2-59 | −193.94 | 42.90 |
| CO2-52 | −198.84 | 41.74 |

¹The number at the end of each input feature indicates the time in minutes from the current time.
²The mean and standard deviation of the permutation feature importance after five rounds of permutation are shown.

TABLE 6
Permutation importance of the input features for the LSTM
model for the five time windows in the shopping mall.
Real Time Prediction

| Input features¹ | Importance mean | Importance standard deviation |
|---|---|---|
| RH | 6189.09 | 301.90 |
| PM10 | 6129.76 | 645.31 |
| PM2.5 | 3927.30 | 296.02 |
| CO2 | 2557.51 | 177.24 |
| Temperature | 2217.54 | 154.56 |
| TVOC | 177.16 | 260.40 |

¹The mean and standard deviation of the permutation feature
importance after five rounds of permutation are shown.

| | | |
|---|---|---|
| Input features^{1,2} | Importance mean | Importance standard deviation |

10-5 Time Window

| | | |
|---|---|---|
| RH-0 | 1161.35 | 275.93 |
| CO2-0 | 1146.13 | 299.44 |
| RH-8 | 703.76 | 161.99 |
| RH-1 | 657.07 | 225.89 |
| Temperature-4 | 599.24 | 98.60 |
| RH-7 | 590.30 | 189.94 |
| RH-9 | 474.59 | 167.44 |
| TVOC-7 | 285.08 | 172.26 |
| CO2-1 | 277.50 | 158.35 |
| PM2.5-0 | 273.34 | 135.75 |
| TVOC-9 | 259.75 | 123.15 |
| Temperature-9 | 255.18 | 216.45 |
| PM2.5-9 | 239.75 | 98.81 |
| PM2.5-1 | 239.00 | 129.29 |
| PM10-1 | 224.05 | 100.24 |
| PM10-3 | 214.15 | 69.81 |
| PM2.5-5 | 134.62 | 166.62 |
| PM10-2 | 120.22 | 86.01 |
| PM2.5-3 | 119.88 | 116.97 |
| PM10-0 | 119.53 | 95.20 |
| PM2.5-4 | 106.97 | 140.25 |
| RH-5 | 106.97 | 184.78 |
| PM2.5-2 | 106.84 | 80.52 |
| TVOC-8 | 96.32 | 90.30 |
| PM10-4 | 92.43 | 143.91 |
| TVOC-0 | 71.61 | 105.79 |
| PM10-8 | 63.01 | 53.99 |
| PM2.5-8 | 46.08 | 89.88 |
| CO2-7 | 21.03 | 56.64 |
| CO2-2 | 18.09 | 144.00 |
| PM10-7 | 12.42 | 53.33 |
| PM10-6 | −1.50 | 114.80 |
| Temperature-8 | −19.59 | 177.82 |
| PM2.5-6 | −31.13 | 176.20 |
| RH-3 | −44.51 | 120.39 |
| RH-4 | −62.12 | 145.73 |
| TVOC-1 | −74.21 | 101.58 |
| RH-6 | −87.24 | 149.40 |
| RH-2 | −106.36 | 109.37 |
| PM10-5 | −106.84 | 148.51 |
| PM2.5-7 | −116.39 | 104.02 |
| CO2-6 | −132.51 | 60.94 |
| CO2-3 | −198.11 | 96.63 |
| TVOC-6 | −203.16 | 90.67 |
| PM10-9 | −212.04 | 94.16 |
| CO2-8 | −246.99 | 81.62 |
| Temperature-3 | −262.89 | 74.27 |
| CO2-4 | −277.78 | 65.38 |
| TVOC-5 | −283.65 | 123.71 |
| TVOC-4 | −294.71 | 65.95 |
| CO2-9 | −378.47 | 149.09 |
| TVOC-2 | −444.55 | 195.48 |
| CO2-5 | −537.12 | 80.91 |
| Temperature-2 | −625.46 | 135.06 |
| TVOC-3 | −644.30 | 128.79 |
| Temperature-0 | −713.66 | 400.35 |
| Temperature-6 | −813.53 | 157.81 |
| Temperature-5 | −819.34 | 175.93 |
| Temperature-7 | −955.26 | 175.83 |
| Temperature-1 | −960.79 | 199.23 |

30-15 Time Window

| | | |
|---|---|---|
| CO2-0 | 1672.55 | 158.03 |
| RH-27 | 788.60 | 96.51 |
| RH-18 | 785.32 | 77.59 |
| CO2-1 | 720.89 | 113.34 |
| Temperature-8 | 688.87 | 145.25 |
| Temperature-5 | 665.39 | 164.15 |
| TVOC-19 | 623.42 | 56.96 |
| Temperature-7 | 598.33 | 124.54 |
| RH-19 | 591.06 | 80.40 |
| Temperature-9 | 577.68 | 170.32 |
| RH-28 | 569.67 | 81.85 |
| Temperature-23 | 521.23 | 111.10 |
| RH-8 | 515.00 | 59.48 |
| RH-16 | 508.04 | 115.52 |
| RH-24 | 475.79 | 74.48 |
| RH-6 | 472.97 | 38.16 |
| RH-26 | 465.06 | 74.62 |
| RH-25 | 462.35 | 58.98 |
| RH-29 | 460.41 | 100.83 |
| CO2-2 | 433.72 | 66.07 |
| TVOC-21 | 427.94 | 39.17 |
| CO2-3 | 422.09 | 46.24 |
| RH-10 | 416.61 | 85.55 |
| RH-20 | 409.04 | 52.28 |
| RH-22 | 406.85 | 47.60 |
| Temperature-6 | 391.33 | 159.59 |
| Temperature-12 | 383.61 | 85.10 |
| Temperature-26 | 375.88 | 152.15 |
| CO2-4 | 374.79 | 46.62 |
| RH-23 | 359.18 | 62.98 |
| TVOC-16 | 341.87 | 29.60 |
| Temperature-11 | 319.32 | 114.78 |
| RH-1 | 316.20 | 97.99 |
| Temperature-10 | 295.25 | 177.49 |
| PM2.5-21 | 295.13 | 19.20 |
| CO2-5 | 292.08 | 52.79 |
| RH-15 | 268.51 | 111.36 |
| CO2-19 | 260.97 | 51.75 |
| RH-21 | 259.65 | 63.73 |
| Temperature-3 | 247.19 | 110.05 |
| CO2-27 | 241.52 | 76.53 |
| RH-17 | 240.74 | 78.78 |
| CO2-11 | 240.62 | 38.11 |
| RH-7 | 238.42 | 88.89 |
| CO2-12 | 229.06 | 30.93 |
| TVOC-29 | 228.41 | 85.91 |
| PM10-1 | 223.35 | 30.39 |
| Temperature-4 | 215.92 | 88.64 |
| TVOC-18 | 212.83 | 42.82 |
| PM2.5-25 | 212.22 | 37.95 |
| Temperature-0 | 204.24 | 108.51 |
| Temperature-27 | 196.92 | 142.53 |
| CO2-21 | 196.48 | 59.09 |
| Temperature-18 | 195.39 | 135.12 |
| RH-11 | 192.18 | 72.08 |
| CO2-15 | 191.02 | 77.46 |
| CO2-25 | 184.66 | 77.99 |
| RH-13 | 184.01 | 63.43 |
| Temperature-2 | 183.69 | 73.83 |
| PM2.5-27 | 180.33 | 42.02 |
| TVOC-22 | 179.59 | 63.91 |
| PM10-13 | 177.54 | 44.19 |
| Temperature-14 | 175.61 | 34.90 |
| TVOC-17 | 170.28 | 45.92 |
| PM10-29 | 167.17 | 36.41 |
| TVOC-27 | 165.30 | 47.40 |
| PM10-18 | 164.59 | 23.23 |
| CO2-10 | 158.83 | 76.84 |
| PM2.5-23 | 158.59 | 30.22 |
| TVOC-12 | 150.71 | 68.15 |
| PM2.5-26 | 141.95 | 20.20 |
| TVOC-2 | 139.22 | 72.82 |
| CO2-17 | 139.05 | 40.90 |
| PM2.5-29 | 138.27 | 43.59 |
| TVOC-23 | 137.97 | 48.17 |
| CO2-9 | 135.45 | 41.66 |
| PM2.5-5 | 133.14 | 36.92 |
| PM2.5-19 | 133.09 | 39.09 |
| Temperature-22 | 128.35 | 99.96 |
| PM2.5-20 | 128.35 | 16.15 |
| TVOC-20 | 128.23 | 48.32 |
| Temperature-19 | 122.13 | 135.86 |
| PM2.5-17 | 121.73 | 28.97 |
| CO2-13 | 119.53 | 60.90 |
| RH-5 | 119.26 | 62.43 |
| PM10-0 | 119.24 | 36.25 |
| PM10-16 | 116.00 | 36.90 |
| Temperature-16 | 115.91 | 110.23 |
| CO2-26 | 115.90 | 63.72 |
| CO2-24 | 115.19 | 50.41 |
| TVOC-24 | 114.73 | 55.15 |
| TVOC-1 | 111.69 | 36.76 |
| Temperature-25 | 103.88 | 122.47 |
| CO2-29 | 103.40 | 69.76 |
| TVOC-14 | 101.55 | 58.21 |
| RH-4 | 98.84 | 44.70 |
| TVOC-9 | 96.18 | 49.61 |
| TVOC-15 | 93.93 | 40.55 |
| Temperature-1 | 89.68 | 92.51 |
| PM2.5-22 | 88.82 | 15.41 |
| CO2-18 | 87.18 | 64.50 |
| RH-0 | 70.88 | 117.23 |
| PM10-28 | 70.65 | 39.12 |
| PM10-15 | 66.92 | 51.67 |
| PM10-12 | 66.30 | 48.73 |
| RH-12 | 59.41 | 64.41 |
| PM10-17 | 55.89 | 44.28 |
| PM2.5-15 | 54.14 | 36.78 |
| PM2.5-28 | 37.49 | 23.63 |
| PM10-10 | 34.87 | 47.83 |
| PM10-14 | 30.62 | 58.35 |
| Temperature-13 | 25.34 | 70.94 |
| PM10-2 | 22.06 | 49.52 |
| CO2-22 | 21.04 | 72.48 |
| PM10-9 | 8.55 | 19.02 |
| PM10-27 | 5.64 | 64.21 |
| PM2.5-8 | 2.29 | 29.45 |
| RH-9 | 0.97 | 84.90 |
| TVOC-28 | 0.56 | 64.15 |
| PM2.5-24 | −7.17 | 19.81 |
| RH-2 | −9.97 | 82.74 |
| PM2.5-0 | −15.45 | 45.76 |
| TVOC-5 | −18.90 | 50.96 |
| PM2.5-4 | −22.15 | 41.34 |
| TVOC-13 | −28.93 | 78.41 |
| PM2.5-14 | −30.46 | 40.31 |
| PM10-19 | −30.66 | 34.27 |
| RH-14 | −30.92 | 49.52 |
| CO2-16 | −38.95 | 76.67 |
| TVOC-25 | −40.15 | 64.42 |
| PM2.5-11 | −41.88 | 41.26 |
| TVOC-11 | −46.64 | 74.03 |
| TVOC-7 | −50.32 | 44.06 |
| TVOC-3 | −55.96 | 24.97 |
| CO2-28 | −56.29 | 74.48 |
| PM2.5-18 | −59.62 | 25.87 |
| CO2-23 | −69.17 | 55.26 |
| PM2.5-6 | −71.27 | 44.78 |
| CO2-20 | −75.95 | 56.47 |
| PM2.5-3 | −83.04 | 44.02 |
| Temperature-24 | −87.88 | 112.55 |
| PM2.5-16 | −90.84 | 55.34 |
| RH-3 | −92.46 | 97.08 |
| Temperature-21 | −93.10 | 18.33 |
| PM2.5-7 | −95.12 | 48.45 |
| TVOC-6 | −99.83 | 46.64 |
| TVOC-4 | −105.15 | 19.34 |
| PM10-11 | −109.32 | 43.30 |
| Temperature-17 | −110.29 | 107.21 |
| PM10-22 | −115.02 | 76.88 |
| TVOC-26 | −117.06 | 55.99 |
| PM2.5-12 | −120.85 | 28.98 |
| CO2-7 | −135.58 | 48.03 |
| CO2-14 | −139.05 | 68.38 |
| PM2.5-1 | −146.85 | 27.58 |
| PM10-25 | −146.94 | 37.44 |
| PM10-4 | −167.45 | 47.19 |
| PM10-8 | −175.54 | 47.67 |
| TVOC-10 | −176.28 | 68.73 |
| PM10-5 | −178.48 | 30.36 |
| PM10-24 | −181.37 | 73.52 |
| PM10-7 | −181.63 | 48.19 |
| CO2-8 | −184.89 | 66.27 |
| PM2.5-9 | −190.05 | 11.95 |
| PM10-3 | −190.51 | 49.85 |
| PM10-6 | −198.47 | 47.88 |
| PM2.5-10 | −202.25 | 31.53 |
| PM2.5-13 | −208.24 | 38.88 |
| CO2-6 | −208.89 | 37.04 |
| PM10-20 | −211.30 | 55.56 |
| Temperature-29 | −229.47 | 205.81 |
| PM10-23 | −230.89 | 82.63 |
| TVOC-8 | −237.78 | 40.41 |
| PM2.5-2 | −244.21 | 35.13 |
| PM10-21 | −251.37 | 46.79 |
| PM10-26 | −271.13 | 98.24 |
| Temperature-28 | −275.13 | 179.21 |
| TVOC-0 | −291.24 | 37.86 |
| Temperature-15 | −367.72 | 72.99 |
| Temperature-20 | −406.75 | 70.95 |

60-30 Time Window

| | | |
|---|---|---|
| Temperature-58 | 211.91 | 46.55 |
| PM10-0 | 206.69 | 16.11 |
| Temperature-57 | 204.99 | 29.93 |
| Temperature-47 | 204.91 | 22.68 |
| Temperature-59 | 204.87 | 30.47 |
| Temperature-56 | 200.69 | 38.76 |
| PM2.5-0 | 182.90 | 18.22 |
| Temperature-30 | 168.64 | 68.54 |
| Temperature-51 | 166.57 | 45.83 |
| Temperature-52 | 166.27 | 20.38 |
| RH-0 | 166.12 | 38.61 |
| Temperature-28 | 162.90 | 48.40 |
| Temperature-27 | 162.42 | 44.96 |
| Temperature-26 | 157.86 | 50.38 |
| Temperature-43 | 157.42 | 34.83 |
| Temperature-25 | 156.60 | 70.66 |
| Temperature-29 | 155.08 | 45.26 |
| Temperature-50 | 153.93 | 55.44 |
| Temperature-39 | 153.86 | 57.12 |
| Temperature-33 | 153.16 | 38.37 |
| Temperature-42 | 152.93 | 44.38 |
| Temperature-55 | 152.86 | 52.50 |
| Temperature-31 | 150.78 | 56.72 |
| Temperature-8 | 150.27 | 20.08 |
| RH-58 | 148.01 | 31.97 |
| RH-1 | 146.60 | 40.93 |
| Temperature-54 | 142.26 | 40.47 |
| Temperature-0 | 139.56 | 44.13 |
| RH-3 | 137.37 | 26.19 |
| Temperature-45 | 136.85 | 20.48 |
| Temperature-48 | 136.30 | 17.35 |
| PM2.5-1 | 135.30 | 26.52 |
| Temperature-44 | 134.82 | 8.94 |
| TVOC-59 | 134.26 | 27.10 |
| RH-59 | 133.37 | 35.40 |
| Temperature-34 | 133.33 | 26.40 |
| Temperature-41 | 130.48 | 48.09 |
| Temperature-46 | 128.00 | 20.27 |
| Temperature-53 | 124.81 | 45.16 |
| PM10-4 | 122.89 | 12.45 |
| Temperature-49 | 122.52 | 37.06 |
| RH-57 | 121.18 | 35.22 |
| Temperature-20 | 120.41 | 43.99 |
| PM2.5-6 | 119.85 | 10.17 |
| Temperature-38 | 119.74 | 37.13 |
| RH-2 | 118.81 | 33.28 |
| Temperature-19 | 118.59 | 47.95 |
| PM10-7 | 117.03 | 20.17 |
| PM10-17 | 116.77 | 6.63 |
| Temperature-23 | 114.51 | 33.67 |
| Temperature-24 | 113.33 | 53.06 |
| Temperature-16 | 112.96 | 52.94 |
| Temperature-9 | 110.25 | 25.95 |
| RH-6 | 108.33 | 53.82 |
| PM10-3 | 107.81 | 16.19 |
| Temperature-3 | 106.33 | 33.03 |
| RH-56 | 106.29 | 28.02 |
| PM2.5-3 | 105.62 | 30.86 |
| Temperature-7 | 103.51 | 44.60 |
| PM2.5-13 | 101.70 | 20.37 |
| PM10-5 | 101.66 | 14.56 |
| Temperature-4 | 101.51 | 23.14 |
| PM2.5-8 | 100.81 | 9.65 |
| Temperature-21 | 100.25 | 49.36 |
| TVOC-49 | 99.33 | 42.06 |
| RH-4 | 98.77 | 36.01 |
| PM2.5-10 | 98.25 | 25.82 |
| TVOC-28 | 96.03 | 30.25 |
| PM10-13 | 96.03 | 8.10 |
| PM10-12 | 94.40 | 10.40 |
| Temperature-17 | 94.29 | 45.17 |
| PM10-14 | 93.51 | 13.92 |
| TVOC-55 | 93.47 | 33.72 |
| PM2.5-4 | 91.36 | 12.07 |
| PM2.5-7 | 91.03 | 22.86 |
| PM2.5-5 | 90.14 | 24.14 |
| PM2.5-2 | 89.51 | 22.53 |
| TVOC-48 | 89.43 | 33.22 |
| Temperature-6 | 89.32 | 26.77 |
| Temperature-15 | 89.32 | 56.54 |
| PM10-6 | 89.14 | 33.80 |
| PM10-59 | 88.91 | 45.06 |
| PM10-58 | 87.06 | 36.99 |
| RH-5 | 86.06 | 37.46 |
| TVOC-27 | 85.47 | 24.34 |
| Temperature-13 | 84.65 | 37.73 |
| Temperature-22 | 83.99 | 42.32 |
| PM10-10 | 83.91 | 19.63 |
| PM10-9 | 83.69 | 17.06 |
| Temperature-32 | 81.25 | 27.01 |
| PM2.5-11 | 81.21 | 25.17 |
| TVOC-25 | 80.02 | 11.13 |
| PM2.5-9 | 79.91 | 28.62 |
| Temperature-5 | 78.54 | 37.36 |
| TVOC-26 | 78.54 | 27.28 |
| Temperature-36 | 78.43 | 46.65 |
| RH-53 | 77.69 | 17.63 |
| Temperature-2 | 77.69 | 25.57 |
| TVOC-54 | 77.58 | 19.96 |
| RH-7 | 77.43 | 41.13 |
| TVOC-50 | 76.10 | 29.67 |
| PM10-57 | 75.91 | 38.31 |
| TVOC-30 | 74.84 | 25.15 |
| TVOC-51 | 74.84 | 39.99 |
| TVOC-58 | 74.50 | 25.53 |
| TVOC-52 | 74.02 | 19.39 |
| Temperature-14 | 73.98 | 38.03 |
| RH-15 | 73.91 | 13.21 |
| TVOC-57 | 72.65 | 30.59 |
| RH-55 | 72.61 | 18.06 |
| RH-9 | 71.61 | 25.70 |
| Temperature-10 | 71.50 | 25.32 |
| TVOC-24 | 71.39 | 39.83 |
| PM2.5-54 | 70.46 | 34.37 |
| Temperature-11 | 70.13 | 26.64 |
| PM10-18 | 70.02 | 21.10 |
| PM10-1 | 69.95 | 7.04 |
| PM2.5-14 | 69.06 | 15.73 |
| Temperature-37 | 68.54 | 43.70 |
| RH-11 | 67.06 | 46.25 |
| PM10-11 | 67.02 | 20.87 |
| Temperature-35 | 65.95 | 36.82 |
| PM2.5-16 | 65.91 | 14.15 |
| PM2.5-12 | 65.61 | 23.01 |
| RH-12 | 65.57 | 22.58 |
| TVOC-20 | 65.57 | 24.15 |
| PM2.5-51 | 64.57 | 36.13 |
| TVOC-31 | 63.28 | 23.99 |
| Temperature-18 | 62.94 | 31.61 |
| RH-54 | 62.80 | 24.08 |
| Temperature-40 | 62.09 | 19.45 |
| TVOC-47 | 61.94 | 9.51 |
| PM2.5-56 | 61.61 | 31.28 |
| PM10-15 | 61.28 | 13.51 |
| TVOC-23 | 61.05 | 30.10 |
| PM10-2 | 58.83 | 9.14 |
| RH-46 | 57.61 | 28.14 |
| TVOC-53 | 56.65 | 25.77 |
| PM2.5-48 | 56.42 | 34.34 |
| PM2.5-19 | 55.68 | 13.95 |
| RH-13 | 55.50 | 23.72 |
| TVOC-22 | 53.72 | 9.91 |
| TVOC-18 | 53.61 | 13.67 |
| RH-48 | 53.02 | 21.60 |
| Temperature-1 | 53.02 | 23.52 |
| CO2-38 | 52.90 | 30.10 |
| RH-8 | 52.53 | 37.11 |
| PM10-19 | 52.46 | 10.51 |
| RH-52 | 52.27 | 36.72 |
| TVOC-21 | 52.13 | 26.37 |
| PM2.5-59 | 51.90 | 32.99 |
| TVOC-29 | 51.35 | 30.60 |
| TVOC-34 | 50.98 | 45.91 |
| TVOC-4 | 50.57 | 34.48 |
| Temperature-12 | 50.46 | 25.89 |
| PM2.5-55 | 49.50 | 25.78 |
| PM10-46 | 47.20 | 33.61 |
| CO2-0 | 46.75 | 23.84 |
| TVOC-32 | 45.72 | 23.58 |
| PM10-54 | 45.64 | 13.45 |
| PM10-16 | 45.53 | 19.94 |
| TVOC-35 | 44.79 | 31.51 |
| PM2.5-15 | 44.46 | 23.66 |
| PM10-8 | 43.35 | 12.76 |
| PM2.5-50 | 42.31 | 13.47 |
| PM2.5-17 | 41.98 | 15.63 |
| PM10-27 | 41.46 | 23.80 |
| PM10-30 | 41.23 | 13.50 |
| RH-14 | 40.60 | 42.14 |
| PM2.5-21 | 40.05 | 12.53 |
| PM10-56 | 39.46 | 51.10 |
| CO2-50 | 39.12 | 26.20 |
| RH-50 | 38.94 | 21.82 |
| PM10-45 | 38.23 | 31.28 |
| PM2.5-28 | 37.90 | 10.20 |
| TVOC-19 | 37.53 | 14.42 |
| RH-28 | 37.53 | 13.39 |
| RH-29 | 36.90 | 24.87 |
| PM10-47 | 36.86 | 7.69 |
| RH-31 | 36.42 | 36.63 |
| CO2-40 | 36.12 | 15.52 |
| CO2-59 | 36.12 | 44.91 |
| TVOC-33 | 35.83 | 22.57 |
| PM10-53 | 34.45 | 16.49 |
| PM2.5-53 | 34.31 | 16.35 |
| RH-10 | 34.16 | 37.96 |
| PM2.5-25 | 34.08 | 7.24 |
| RH-42 | 33.94 | 17.36 |
| TVOC-1 | 33.94 | 28.02 |
| PM10-20 | 32.64 | 10.09 |
| TVOC-56 | 32.56 | 16.64 |
| PM2.5-33 | 32.49 | 13.49 |
| CO2-57 | 31.82 | 55.97 |
| PM10-29 | 31.60 | 16.43 |
| PM10-55 | 30.42 | 32.91 |
| TVOC-3 | 29.82 | 33.43 |
| TVOC-46 | 29.34 | 23.52 |
| PM10-38 | 29.01 | 7.56 |
| RH-16 | 28.71 | 20.67 |
| PM2.5-58 | 28.27 | 29.79 |
| TVOC-8 | 28.23 | 41.55 |
| PM2.5-20 | 28.19 | 15.00 |
| TVOC-9 | 28.01 | 11.81 |
| PM2.5-32 | 27.49 | 18.18 |
| PM2.5-57 | 27.30 | 25.36 |
| PM10-52 | 27.23 | 6.19 |
| CO2-49 | 26.45 | 30.38 |
| RH-24 | 25.41 | 26.94 |
| PM2.5-35 | 25.27 | 17.91 |
| CO2-47 | 25.19 | 22.71 |
| PM10-50 | 24.86 | 28.86 |
| TVOC-6 | 24.56 | 53.21 |
| PM2.5-18 | 24.19 | 32.42 |
| PM2.5-23 | 23.86 | 12.40 |
| CO2-48 | 23.75 | 14.69 |
| CO2-46 | 23.12 | 23.31 |
| PM2.5-47 | 23.01 | 40.52 |
| TVOC-43 | 22.97 | 22.22 |
| TVOC-37 | 22.78 | 27.41 |
| TVOC-40 | 22.64 | 13.98 |
| PM2.5-34 | 22.60 | 14.51 |
| CO2-52 | 22.12 | 29.39 |
| PM10-22 | 21.38 | 7.38 |
| CO2-53 | 21.34 | 11.23 |
| TVOC-38 | 21.34 | 24.72 |
| TVOC-11 | 21.34 | 41.93 |
| PM2.5-52 | 20.27 | 9.44 |
| CO2-1 | 19.60 | 21.32 |
| CO2-37 | 19.30 | 20.35 |
| TVOC-14 | 19.15 | 24.89 |
| PM10-49 | 18.64 | 13.13 |
| PM10-42 | 18.52 | 18.44 |
| CO2-58 | 18.23 | 60.72 |
| PM2.5-45 | 17.49 | 22.36 |
| PM10-31 | 17.15 | 20.40 |
| PM2.5-49 | 16.75 | 26.36 |
| CO2-44 | 16.08 | 28.92 |
| PM10-48 | 15.56 | 24.83 |
| TVOC-39 | 15.37 | 24.13 |
| RH-36 | 15.37 | 24.01 |
| RH-27 | 15.15 | 16.88 |
| RH-51 | 14.08 | 17.03 |
| RH-22 | 13.97 | 34.88 |
| PM2.5-46 | 13.82 | 11.23 |
| CO2-51 | 13.63 | 50.67 |
| TVOC-2 | 13.19 | 21.06 |
| PM10-34 | 12.78 | 6.56 |
| CO2-42 | 12.08 | 29.36 |
| CO2-39 | 11.89 | 31.15 |
| PM2.5-31 | 11.19 | 9.96 |
| PM2.5-30 | 11.15 | 21.07 |
| RH-43 | 11.08 | 9.94 |
| PM2.5-44 | 11.00 | 31.08 |
| RH-37 | 10.89 | 10.92 |
| RH-21 | 10.56 | 36.13 |
| TVOC-42 | 9.34 | 24.50 |
| PM10-44 | 9.04 | 29.72 |
| PM10-21 | 8.93 | 18.52 |
| RH-49 | 8.63 | 12.68 |
| PM10-28 | 8.22 | 15.48 |
| PM2.5-37 | 8.15 | 5.36 |
| PM10-25 | 7.71 | 17.04 |
| PM10-23 | 7.67 | 13.36 |
| PM10-37 | 6.89 | 12.09 |
| CO2-45 | 6.34 | 27.08 |
| CO2-55 | 5.93 | 62.19 |
| RH-35 | 5.19 | 12.58 |
| RH-18 | 4.63 | 22.80 |
| RH-40 | 4.59 | 31.66 |
| TVOC-10 | 4.52 | 34.49 |
| TVOC-7 | 4.11 | 17.61 |
| RH-47 | 4.00 | 17.85 |
| CO2-34 | 2.37 | 24.20 |
| TVOC-5 | 2.11 | 29.84 |
| PM2.5-22 | 2.04 | 26.41 |
| PM10-24 | 1.19 | 11.80 |
| CO2-3 | 1.19 | 10.37 |
| PM10-43 | 1.15 | 11.47 |
| TVOC-36 | 1.15 | 24.97 |
| RH-39 | 0.15 | 31.72 |
| PM2.5-36 | 0.04 | 17.18 |
| TVOC-0 | −0.15 | 29.09 |
| CO2-36 | −1.00 | 20.70 |
| CO2-41 | −1.19 | 37.18 |
| PM10-33 | −1.19 | 15.29 |
| RH-26 | −1.56 | 35.66 |
| CO2-43 | −1.70 | 34.35 |
| TVOC-15 | −2.19 | 16.38 |
| RH-32 | −2.41 | 20.14 |
| RH-41 | −3.22 | 31.36 |
| TVOC-17 | −3.37 | 17.41 |
| RH-17 | −3.85 | 21.15 |
| TVOC-13 | −3.89 | 19.39 |
| RH-44 | −3.96 | 19.66 |
| CO2-56 | −4.11 | 52.66 |
| CO2-27 | −4.41 | 12.30 |
| RH-25 | −5.15 | 39.31 |
| CO2-54 | −5.33 | 56.10 |
| TVOC-45 | −5.85 | 17.30 |
| PM10-26 | −6.56 | 9.50 |
| TVOC-44 | −6.71 | 18.44 |
| PM2.5-42 | −6.78 | 20.97 |
| PM2.5-41 | −7.11 | 14.03 |
| PM10-36 | −7.15 | 12.79 |
| PM2.5-39 | −7.30 | 13.14 |
| PM2.5-40 | −7.41 | 42.25 |
| CO2-23 | −7.74 | 22.74 |
| PM2.5-43 | −7.85 | 15.74 |
| PM10-32 | −8.04 | 20.67 |
| PM2.5-24 | −8.26 | 17.50 |
| CO2-35 | −8.60 | 16.74 |
| CO2-33 | −9.48 | 34.61 |
| RH-34 | −10.41 | 26.28 |
| TVOC-12 | −11.41 | 33.49 |
| RH-23 | −11.89 | 38.13 |
| CO2-21 | −11.89 | 15.53 |
| PM2.5-29 | −11.97 | 15.35 |
| PM2.5-38 | −13.04 | 15.97 |
| PM10-51 | −13.34 | 18.77 |
| PM10-41 | −14.41 | 13.34 |
| RH-30 | −14.63 | 22.54 |
| CO2-18 | −14.82 | 11.59 |
| TVOC-41 | −17.08 | 18.38 |
| TVOC-16 | −17.12 | 18.32 |
| PM10-39 | −17.41 | 23.20 |
| RH-38 | −19.12 | 18.85 |
| PM10-35 | −19.60 | 17.56 |
| CO2-5 | −19.67 | 13.50 |
| RH-20 | −19.82 | 13.42 |
| RH-45 | −21.56 | 8.30 |
| PM2.5-27 | −22.19 | 11.96 |
| CO2-17 | −22.64 | 10.47 |
| RH-33 | −23.86 | 18.72 |
| CO2-19 | −24.71 | 11.10 |
| PM10-40 | −24.90 | 25.39 |
| PM2.5-26 | −26.01 | 14.54 |
| CO2-4 | −26.08 | 18.97 |
| CO2-20 | −28.79 | 18.15 |
| CO2-2 | −31.05 | 21.58 |
| CO2-28 | −31.08 | 9.61 |
| CO2-31 | −32.12 | 21.60 |
| CO2-14 | −32.27 | 19.49 |
| CO2-30 | −32.60 | 23.12 |
| RH-19 | −33.53 | 38.98 |
| CO2-29 | −38.42 | 21.05 |
| CO2-10 | −39.49 | 8.27 |
| CO2-25 | −40.12 | 28.37 |
| CO2-6 | −40.27 | 10.54 |
| CO2-8 | −41.01 | 16.63 |
| CO2-15 | −42.57 | 6.84 |
| CO2-24 | −43.79 | 12.39 |
| CO2-11 | −44.35 | 9.44 |
| CO2-32 | −45.05 | 20.61 |
| CO2-22 | −45.16 | 27.71 |
| CO2-9 | −46.01 | 15.97 |
| CO2-26 | −46.42 | 37.01 |
| CO2-13 | −48.42 | 11.30 |
| CO2-12 | −48.90 | 11.55 |
| CO2-16 | −50.90 | 5.54 |
| CO2-7 | −59.57 | 14.77 |

60-60 Time Window

| | | |
|---|---|---|
| CO2-0 | 160.80 | 54.07 |
| RH-2 | 137.16 | 44.27 |
| Temperature-59 | 136.47 | 62.46 |
| Temperature-58 | 123.95 | 19.84 |
| Temperature-0 | 121.31 | 67.78 |
| RH-4 | 120.24 | 51.03 |
| RH-9 | 111.60 | 29.02 |
| RH-58 | 110.24 | 26.80 |
| PM2.5-0 | 110.04 | 26.28 |
| RH-59 | 108.98 | 22.40 |
| RH-3 | 108.64 | 51.97 |
| RH-11 | 108.35 | 32.97 |
| RH-7 | 106.52 | 36.22 |
| TVOC-36 | 106.50 | 12.08 |
| Temperature-57 | 106.31 | 17.67 |
| RH-1 | 99.42 | 55.21 |
| RH-10 | 98.74 | 19.82 |
| RH-0 | 97.57 | 49.01 |
| PM2.5-4 | 96.71 | 16.29 |
| PM10-0 | 95.69 | 13.13 |
| RH-5 | 94.03 | 35.22 |
| TVOC-37 | 92.44 | 10.76 |
| TVOC-41 | 91.24 | 30.08 |
| Temperature-55 | 89.02 | 11.96 |
| RH-54 | 87.60 | 28.96 |
| RH-17 | 85.94 | 30.08 |
| CO2-1 | 85.41 | 54.36 |
| PM10-8 | 83.96 | 15.62 |
| TVOC-0 | 83.53 | 17.23 |
| Temperature-52 | 82.25 | 17.52 |
| RH-8 | 81.33 | 32.48 |
| PM10-7 | 80.98 | 13.06 |
| PM10-6 | 80.60 | 11.87 |
| TVOC-42 | 78.46 | 14.25 |
| PM10-5 | 77.72 | 7.55 |
| PM10-3 | 77.40 | 18.67 |
| RH-12 | 76.52 | 17.23 |
| PM2.5-3 | 75.64 | 16.06 |
| PM10-4 | 75.62 | 20.62 |
| RH-56 | 73.90 | 33.41 |
| RH-57 | 73.62 | 25.40 |
| PM10-1 | 73.27 | 9.53 |
| PM2.5-11 | 72.42 | 8.17 |
| RH-14 | 72.34 | 18.48 |
| TVOC-38 | 72.32 | 13.77 |
| PM2.5-9 | 70.88 | 13.88 |
| RH-51 | 70.72 | 27.77 |
| RH-15 | 70.21 | 36.29 |
| RH-52 | 70.06 | 20.98 |
| PM2.5-10 | 69.65 | 8.76 |
| CO2-2 | 68.83 | 56.85 |
| PM2.5-1 | 68.20 | 7.47 |
| RH-20 | 66.89 | 14.17 |
| TVOC-43 | 66.55 | 3.01 |
| Temperature-56 | 65.90 | 14.66 |
| RH-47 | 65.59 | 25.56 |
| RH-42 | 65.26 | 26.77 |
| RH-6 | 64.71 | 40.47 |
| CO2-4 | 64.58 | 67.23 |
| RH-55 | 64.40 | 35.65 |
| PM2.5-2 | 63.17 | 18.22 |
| Temperature-9 | 62.07 | 31.72 |
| PM10-10 | 61.46 | 21.33 |
| RH-40 | 60.81 | 10.03 |
| RH-13 | 60.70 | 34.69 |
| PM10-9 | 59.01 | 19.53 |
| Temperature-54 | 58.92 | 8.84 |
| Temperature-51 | 58.10 | 17.74 |
| RH-53 | 57.95 | 20.19 |
| CO2-5 | 57.84 | 59.80 |
| PM2.5-8 | 57.19 | 8.97 |
| Temperature-44 | 57.09 | 26.54 |
| RH-49 | 56.68 | 32.63 |
| CO2-3 | 54.56 | 65.68 |
| TVOC-35 | 54.50 | 14.64 |
| PM2.5-5 | 54.24 | 21.35 |
| PM10-22 | 53.95 | 17.46 |
| RH-32 | 53.54 | 16.31 |
| TVOC-39 | 53.29 | 18.80 |
| TVOC-45 | 52.93 | 14.50 |
| RH-16 | 52.46 | 19.20 |
| TVOC-1 | 52.16 | 13.52 |
| RH-43 | 51.59 | 32.67 |
| Temperature-53 | 50.01 | 30.32 |
| RH-21 | 49.71 | 24.82 |
| RH-18 | 49.48 | 26.50 |
| PM10-11 | 48.95 | 8.72 |
| Temperature-1 | 48.25 | 29.25 |
| TVOC-32 | 48.16 | 21.52 |
| PM2.5-13 | 48.03 | 24.66 |
| RH-31 | 47.60 | 28.04 |
| Temperature-22 | 47.39 | 28.16 |
| RH-36 | 47.06 | 16.72 |
| Temperature-3 | 46.60 | 30.52 |
| TVOC-33 | 46.05 | 15.07 |
| RH-44 | 45.89 | 16.69 |
| TVOC-40 | 45.83 | 8.48 |
| Temperature-11 | 45.69 | 16.84 |
| TVOC-34 | 45.53 | 6.55 |
| TVOC-28 | 44.93 | 11.52 |
| RH-24 | 44.60 | 35.32 |
| Temperature-4 | 44.12 | 26.56 |
| Temperature-21 | 43.60 | 44.40 |
| RH-23 | 43.10 | 31.72 |
| RH-50 | 42.73 | 24.71 |
| PM2.5-6 | 42.69 | 14.21 |
| CO2-6 | 42.40 | 57.15 |
| PM2.5-12 | 42.07 | 21.00 |
| RH-22 | 41.62 | 26.89 |
| Temperature-8 | 41.12 | 31.27 |
| Temperature-17 | 41.05 | 31.94 |
| Temperature-10 | 40.71 | 28.40 |
| Temperature-6 | 40.57 | 32.36 |
| PM2.5-19 | 40.27 | 16.99 |
| RH-39 | 40.23 | 15.30 |
| RH-37 | 39.04 | 21.82 |
| Temperature-49 | 38.57 | 15.97 |
| TVOC-49 | 37.99 | 10.49 |
| RH-25 | 37.17 | 22.92 |
| PM10-2 | 36.69 | 23.92 |
| RH-29 | 36.64 | 23.26 |
| RH-46 | 36.48 | 34.29 |
| CO2-11 | 34.96 | 45.58 |
| RH-27 | 33.94 | 36.03 |
| RH-28 | 33.91 | 28.62 |
| RH-48 | 33.81 | 30.64 |
| PM2.5-7 | 33.58 | 14.42 |
| PM10-12 | 33.56 | 18.25 |
| TVOC-17 | 33.54 | 21.43 |
| PM10-25 | 33.03 | 18.05 |
| TVOC-46 | 33.01 | 14.12 |
| TVOC-44 | 32.70 | 19.67 |
| TVOC-30 | 32.66 | 8.54 |
| RH-19 | 31.49 | 15.97 |
| TVOC-47 | 31.46 | 17.25 |
| TVOC-48 | 30.92 | 24.49 |
| Temperature-50 | 30.56 | 12.41 |
| PM10-13 | 29.96 | 15.89 |
| TVOC-9 | 29.73 | 10.20 |
| RH-35 | 27.77 | 15.88 |
| Temperature-45 | 27.54 | 28.22 |
| TVOC-56 | 26.37 | 17.23 |
| RH-45 | 26.07 | 17.37 |
| TVOC-55 | 26.04 | 23.52 |
| RH-34 | 25.70 | 20.88 |
| RH-41 | 24.84 | 17.25 |
| TVOC-4 | 24.82 | 17.84 |
| CO2-7 | 23.59 | 63.79 |
| TVOC-52 | 22.84 | 15.13 |
| TVOC-10 | 20.45 | 14.53 |
| TVOC-7 | 20.36 | 4.19 |
| TVOC-50 | 19.56 | 18.67 |
| Temperature-2 | 19.19 | 19.65 |
| TVOC-27 | 18.73 | 6.67 |
| PM2.5-33 | 18.63 | 39.20 |
| Temperature-47 | 18.58 | 15.71 |
| RH-26 | 18.21 | 39.17 |
| Temperature-7 | 18.04 | 29.28 |
| Temperature-23 | 17.69 | 43.27 |
| TVOC-12 | 17.64 | 14.87 |
| PM10-14 | 17.48 | 14.90 |
| RH-30 | 17.36 | 25.44 |
| TVOC-6 | 16.16 | 12.77 |
| PM10-36 | 15.39 | 14.56 |
| PM10-41 | 15.13 | 14.54 |
| TVOC-51 | 15.02 | 19.09 |
| TVOC-15 | 14.89 | 22.80 |
| PM2.5-18 | 14.20 | 16.00 |
| RH-38 | 14.13 | 11.21 |
| TVOC-11 | 14.13 | 7.56 |
| PM2.5-31 | 13.30 | 7.17 |
| Temperature-20 | 12.97 | 33.17 |
| PM10-20 | 11.85 | 18.29 |
| Temperature-25 | 11.09 | 61.66 |
| TVOC-25 | 10.67 | 10.40 |
| PM10-18 | 10.64 | 24.75 |
| PM2.5-27 | 9.99 | 14.49 |
| TVOC-26 | 9.86 | 21.60 |
| PM10-23 | 9.12 | 12.07 |
| PM2.5-24 | 8.76 | 24.63 |
| Temperature-5 | 8.73 | 27.39 |
| TVOC-13 | 7.83 | 23.56 |
| PM10-17 | 7.43 | 12.46 |
| PM10-15 | 7.38 | 11.12 |
| PM10-21 | 7.02 | 23.88 |
| Temperature-46 | 6.52 | 18.11 |
| PM2.5-21 | 6.37 | 21.94 |
| CO2-9 | 5.96 | 50.85 |
| PM10-37 | 5.94 | 17.47 |
| TVOC-29 | 5.69 | 5.15 |
| Temperature-48 | 5.46 | 10.68 |
| PM10-16 | 5.29 | 21.76 |
| PM2.5-16 | 4.70 | 34.24 |
| TVOC-5 | 3.77 | 25.74 |
| PM2.5-39 | 2.39 | 23.11 |
| TVOC-54 | 2.33 | 26.10 |
| PM2.5-15 | 1.06 | 12.26 |
| PM10-26 | 0.52 | 22.56 |
| Temperature-15 | 0.48 | 32.30 |
| PM10-44 | 0.38 | 25.96 |
| TVOC-16 | 0.37 | 21.40 |
| TVOC-57 | −0.37 | 21.69 |
| PM2.5-26 | −0.39 | 18.03 |
| PM2.5-35 | −0.61 | 28.20 |
| TVOC-58 | −1.61 | 27.04 |
| RH-33 | −2.33 | 16.00 |
| PM2.5-42 | −2.62 | 17.28 |
| PM2.5-14 | −2.76 | 21.81 |
| PM2.5-20 | −3.39 | 23.96 |
| CO2-14 | −3.46 | 31.01 |
| Temperature-13 | −4.26 | 24.76 |
| PM10-19 | −4.47 | 16.29 |
| PM10-24 | −4.71 | 21.58 |
| TVOC-31 | −4.74 | 14.88 |
| Temperature-19 | −4.79 | 30.34 |
| Temperature-43 | −5.14 | 14.03 |
| TVOC-22 | −5.63 | 17.91 |
| TVOC-2 | −5.83 | 18.11 |
| CO2-12 | −5.99 | 40.49 |
| Temperature-14 | −6.19 | 16.19 |
| TVOC-59 | −6.25 | 15.92 |
| PM2.5-22 | −6.77 | 20.42 |
| PM10-49 | −6.78 | 27.80 |
| PM2.5-46 | −6.83 | 23.69 |
| PM2.5-41 | −7.06 | 22.99 |
| Temperature-18 | −7.06 | 36.35 |
| TVOC-14 | −7.33 | 27.12 |
| PM2.5-17 | −7.56 | 22.40 |
| PM10-40 | −8.06 | 12.83 |
| CO2-15 | −8.13 | 20.52 |
| CO2-13 | −9.91 | 49.75 |
| PM2.5-36 | −10.07 | 12.22 |
| PM2.5-45 | −10.31 | 22.23 |
| PM2.5-59 | −10.99 | 21.57 |
| Temperature-26 | −11.62 | 62.03 |
| PM2.5-34 | −12.12 | 7.52 |
| CO2-44 | −12.73 | 28.18 |
| PM10-29 | −12.74 | 16.99 |
| Temperature-42 | −13.43 | 10.17 |
| TVOC-8 | −14.94 | 17.28 |
| CO2-10 | −15.91 | 52.85 |
| CO2-30 | −16.20 | 27.17 |
| PM10-52 | −16.35 | 22.25 |
| PM2.5-38 | −18.09 | 25.99 |
| PM2.5-28 | −18.24 | 12.43 |
| TVOC-19 | −18.75 | 25.42 |
| Temperature-35 | −20.11 | 41.54 |
| PM10-34 | −20.16 | 11.41 |
| TVOC-18 | −22.55 | 29.00 |
| PM2.5-54 | −22.88 | 24.62 |
| PM2.5-25 | −23.60 | 22.18 |
| PM2.5-58 | −24.36 | 7.39 |
| PM10-51 | −24.67 | 17.85 |
| PM10-32 | −24.99 | 13.50 |
| TVOC-24 | −25.33 | 8.78 |
| CO2-8 | −25.37 | 53.13 |
| PM2.5-44 | −25.50 | 10.10 |
| CO2-34 | −27.12 | 23.62 |
| CO2-18 | −27.59 | 44.56 |
| Temperature-28 | −27.95 | 34.54 |
| TVOC-3 | −28.66 | 9.45 |
| CO2-55 | −29.32 | 60.68 |
| PM10-53 | −29.42 | 9.23 |
| TVOC-21 | −29.43 | 26.94 |
| CO2-52 | −29.63 | 50.77 |
| Temperature-12 | −30.24 | 14.50 |
| CO2-36 | −30.77 | 32.36 |
| Temperature-24 | −30.82 | 30.61 |
| PM2.5-52 | −31.13 | 17.03 |
| Temperature-37 | −31.42 | 18.25 |
| CO2-45 | −31.44 | 37.75 |
| PM10-39 | −32.45 | 17.55 |
| CO2-48 | −32.78 | 51.68 |
| CO2-17 | −33.56 | 45.67 |
| PM10-27 | −34.50 | 15.72 |
| Temperature-39 | −34.65 | 18.70 |
| PM10-50 | −35.91 | 17.88 |
| TVOC-23 | −35.91 | 35.65 |
| PM10-42 | −36.17 | 17.94 |
| Temperature-16 | −36.32 | 40.60 |
| CO2-41 | −37.86 | 32.58 |
| PM2.5-29 | −38.10 | 20.71 |
| PM10-30 | −38.13 | 11.21 |
| CO2-54 | −38.21 | 41.18 |
| PM2.5-32 | −38.37 | 22.25 |
| PM2.5-57 | −38.52 | 16.40 |
| TVOC-53 | −38.97 | 35.42 |
| CO2-40 | −39.20 | 16.02 |
| CO2-53 | −40.25 | 47.20 |
| PM10-58 | −40.73 | 12.15 |
| PM2.5-56 | −41.30 | 15.95 |
| CO2-49 | −41.38 | 46.99 |
| CO2-50 | −42.37 | 50.65 |
| PM10-56 | −42.84 | 16.01 |
| CO2-21 | −43.15 | 43.41 |
| CO2-16 | −43.69 | 45.97 |
| CO2-22 | −43.71 | 38.52 |
| CO2-37 | −44.35 | 20.59 |
| PM2.5-51 | −46.00 | 19.39 |
| CO2-23 | −46.57 | 27.93 |
| Temperature-41 | −47.61 | 16.04 |
| PM2.5-30 | −47.85 | 9.13 |
| Temperature-40 | −48.15 | 21.93 |
| PM10-45 | −48.33 | 19.35 |
| TVOC-20 | −48.48 | 21.31 |
| PM2.5-23 | −49.11 | 43.82 |
| CO2-46 | −49.18 | 45.30 |
| PM10-31 | −49.60 | 10.46 |
| CO2-42 | −50.22 | 43.46 |
| PM2.5-55 | −51.36 | 28.25 |
| CO2-51 | −52.36 | 40.11 |
| Temperature-34 | −53.47 | 28.35 |
| CO2-43 | −53.50 | 22.54 |
| PM10-28 | −53.72 | 15.95 |
| Temperature-33 | −54.26 | 31.15 |
| CO2-38 | −54.49 | 25.45 |
| CO2-59 | −55.70 | 61.42 |
| Temperature-30 | −56.13 | 40.48 |
| PM10-38 | −57.35 | 19.49 |
| PM10-47 | −57.35 | 18.04 |
| PM10-33 | −57.38 | 9.56 |
| CO2-35 | −57.50 | 24.57 |
| CO2-32 | −57.90 | 26.33 |
| PM2.5-47 | −58.73 | 24.82 |
| PM10-57 | −59.21 | 24.19 |
| PM10-46 | −60.24 | 12.35 |
| CO2-28 | −60.29 | 30.18 |
| PM2.5-48 | −60.57 | 15.46 |
| Temperature-38 | −61.32 | 32.13 |
| PM2.5-49 | −61.77 | 28.12 |
| Temperature-36 | −61.83 | 12.54 |
| CO2-25 | −62.12 | 32.40 |
| Temperature-29 | −62.73 | 43.71 |
| PM2.5-37 | −62.91 | 21.45 |
| CO2-29 | −65.56 | 25.38 |
| CO2-31 | −66.32 | 23.70 |
| PM10-35 | −67.41 | 20.69 |
| CO2-20 | −67.97 | 32.10 |
| CO2-39 | −68.17 | 32.01 |
| Temperature-27 | −69.48 | 31.87 |
| PM2.5-40 | −71.03 | 20.15 |
| CO2-57 | −72.09 | 29.46 |
| PM10-59 | −74.61 | 24.51 |
| PM10-48 | −74.81 | 12.74 |
| CO2-58 | −75.21 | 58.48 |
| PM10-54 | −75.50 | 26.42 |
| PM2.5-53 | −76.27 | 22.43 |
| PM2.5-43 | −76.31 | 17.58 |
| CO2-47 | −79.63 | 40.07 |
| Temperature-31 | −84.11 | 42.39 |
| PM10-43 | −84.92 | 28.70 |
| CO2-19 | −85.61 | 42.37 |
| PM10-55 | −87.62 | 18.40 |
| CO2-33 | −87.63 | 34.27 |
| CO2-24 | −88.30 | 29.04 |
| CO2-56 | −88.78 | 36.31 |
| CO2-27 | −92.07 | 35.12 |
| PM2.5-50 | −98.52 | 16.09 |
| Temperature-32 | −101.01 | 24.18 |
| CO2-26 | −101.35 | 33.82 |

¹The number at the end of each input feature indicates the time in minute from the current time.
²The mean and standard deviation of the permutation feature importance after five rounds of permutation are shown.

Prediction of Time Series Data

Lastly, as the trained LSTM model was determined to be the best model for both venue types which are the commercial office and the shopping mall, the model is then applied in the actual monitoring and forecasting operation in Steps 41 and 43 (see FIG. 2), e.g. to be used as the AI model shown in FIG. 1. In Step 41, measured data (which may have different time lengths) at a desired venue is inputted into the best model, and in Step 43, a prediction of concentration of indoor bioaerosols by the best model is generated for the venue.

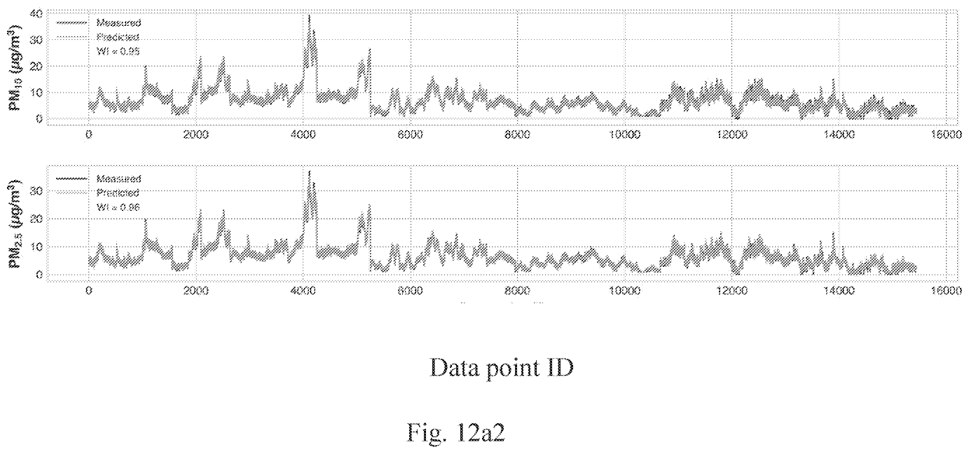

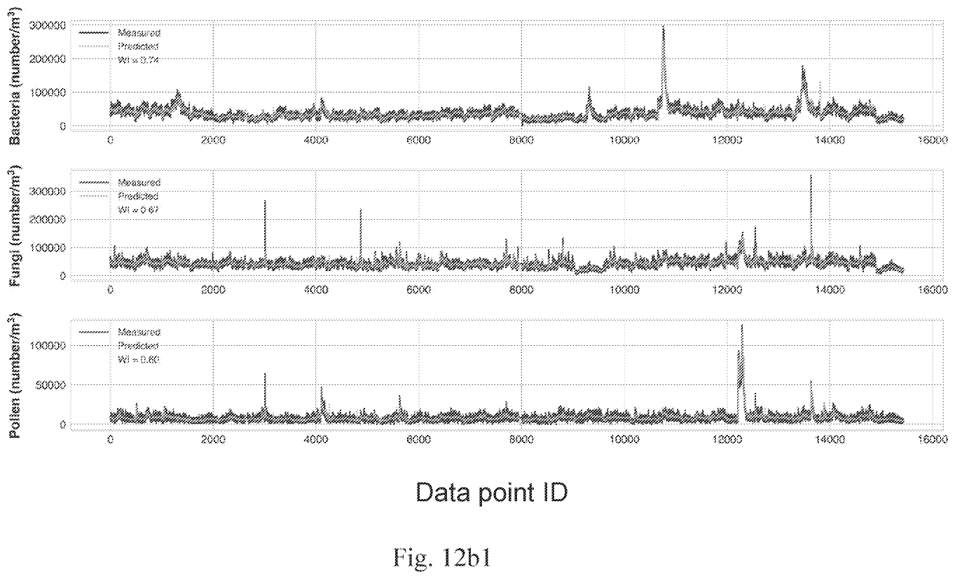

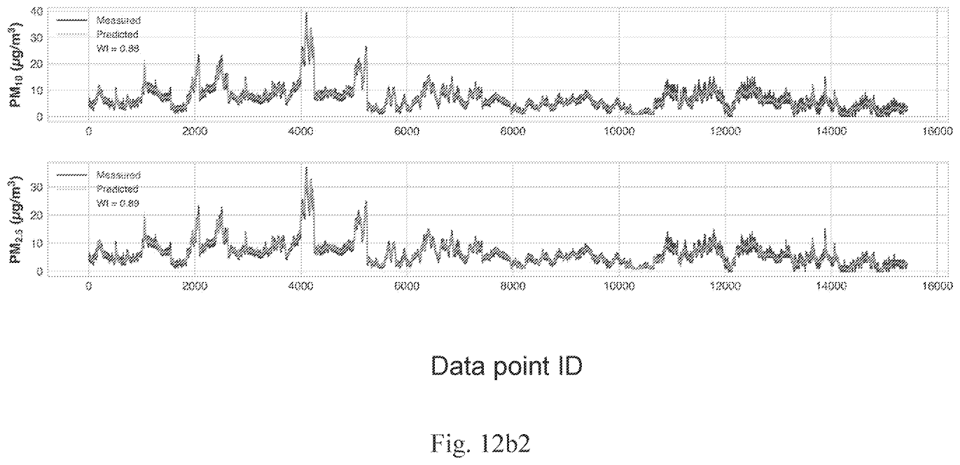

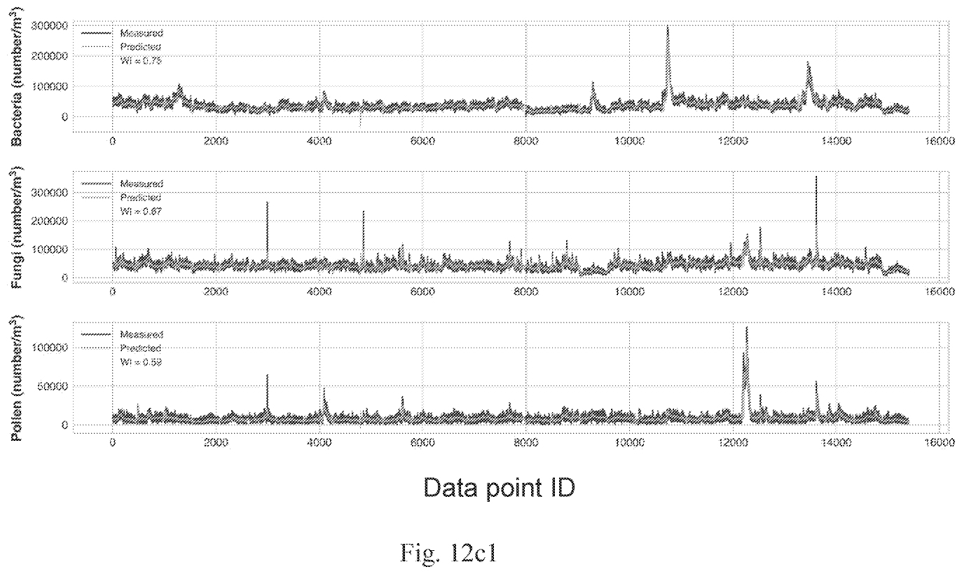

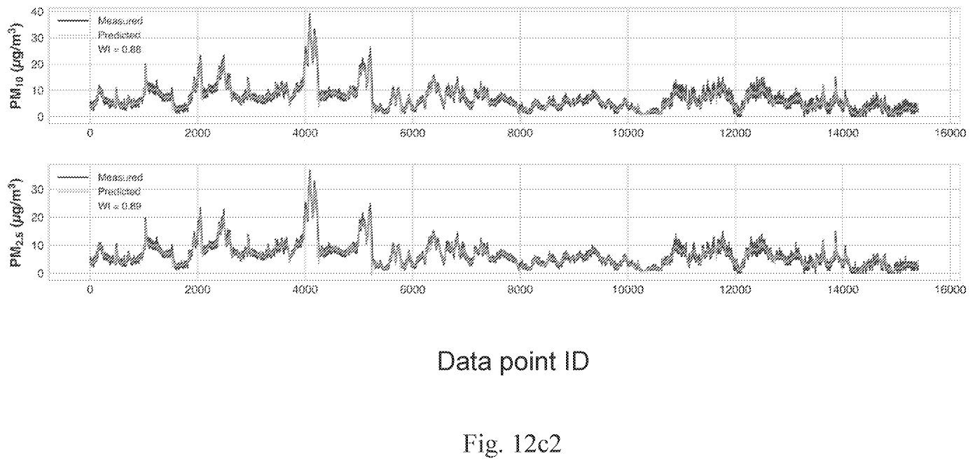

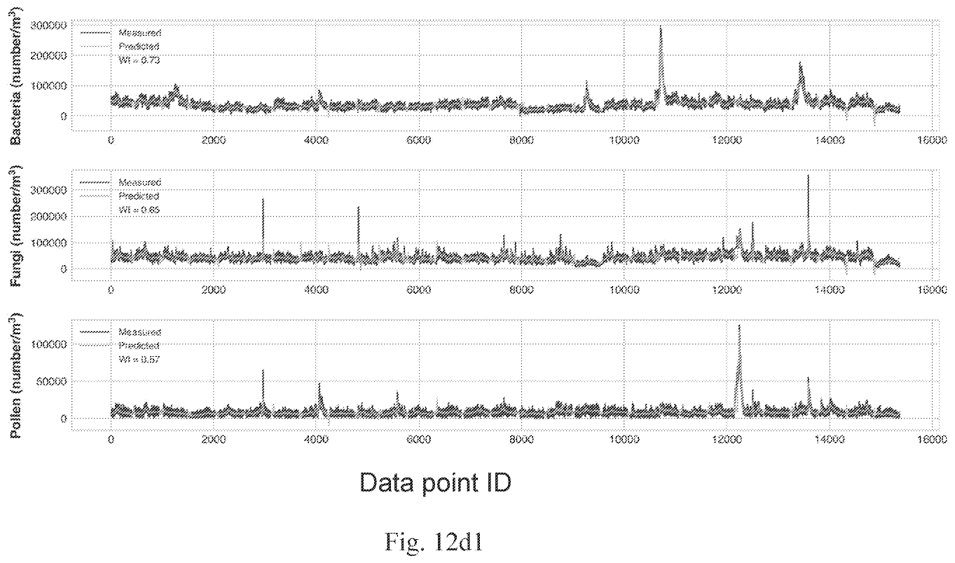

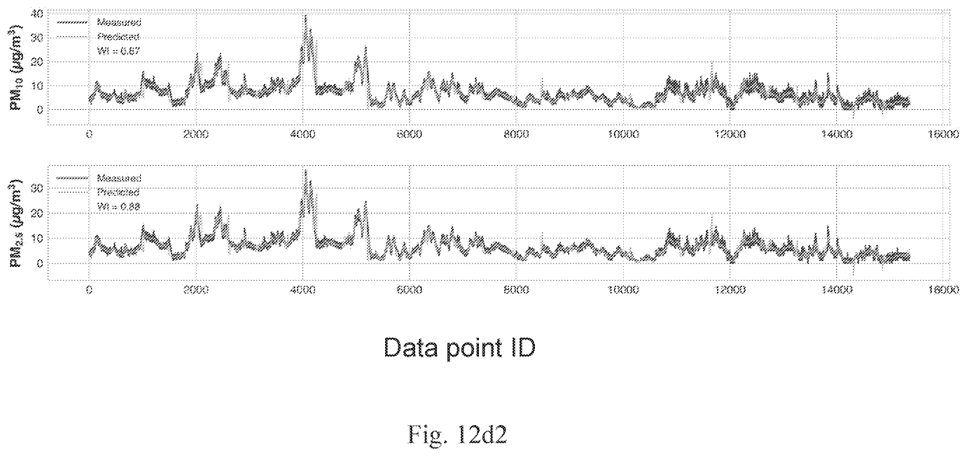

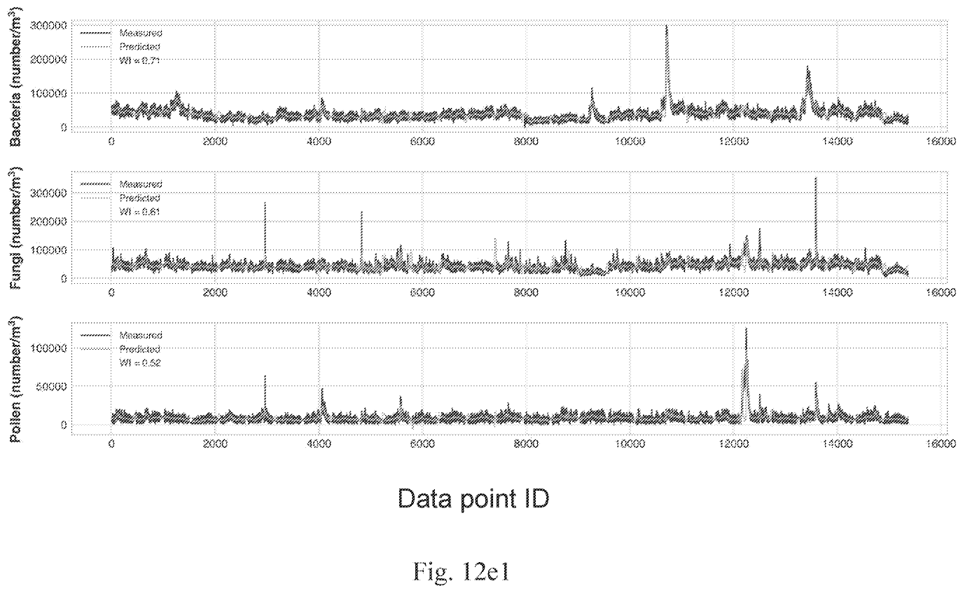

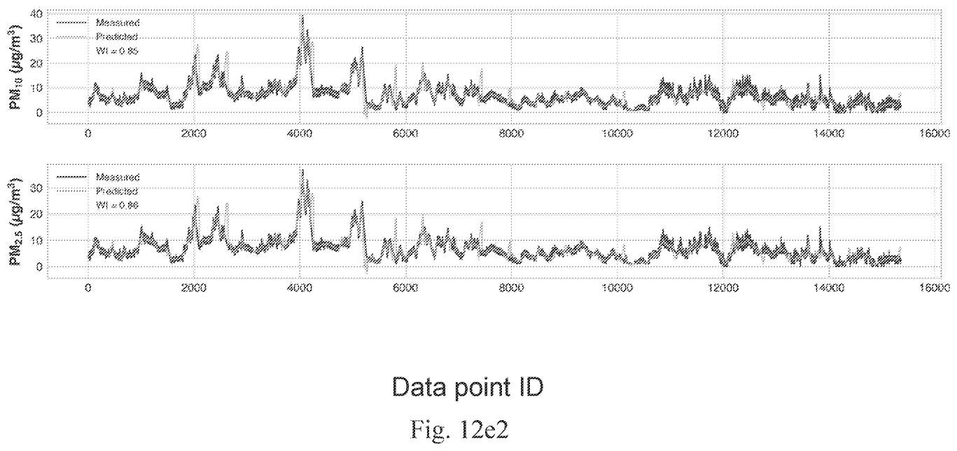

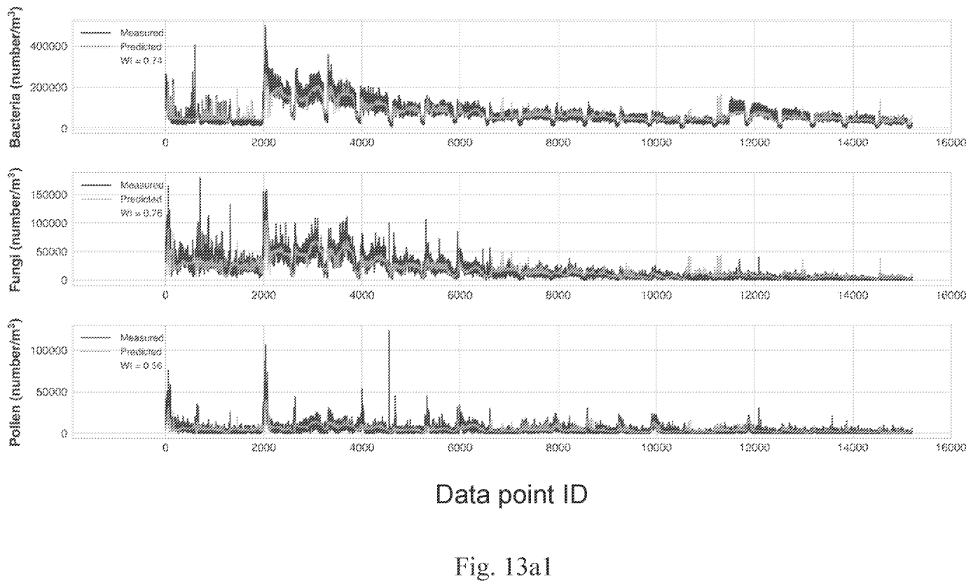

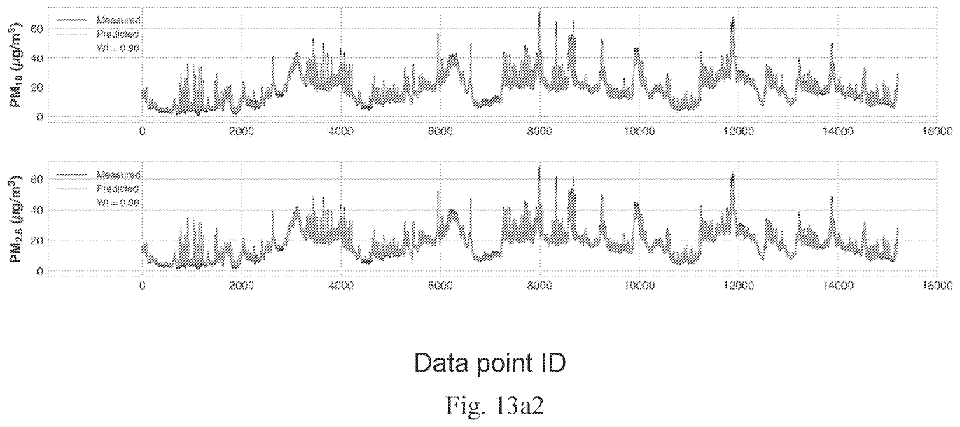

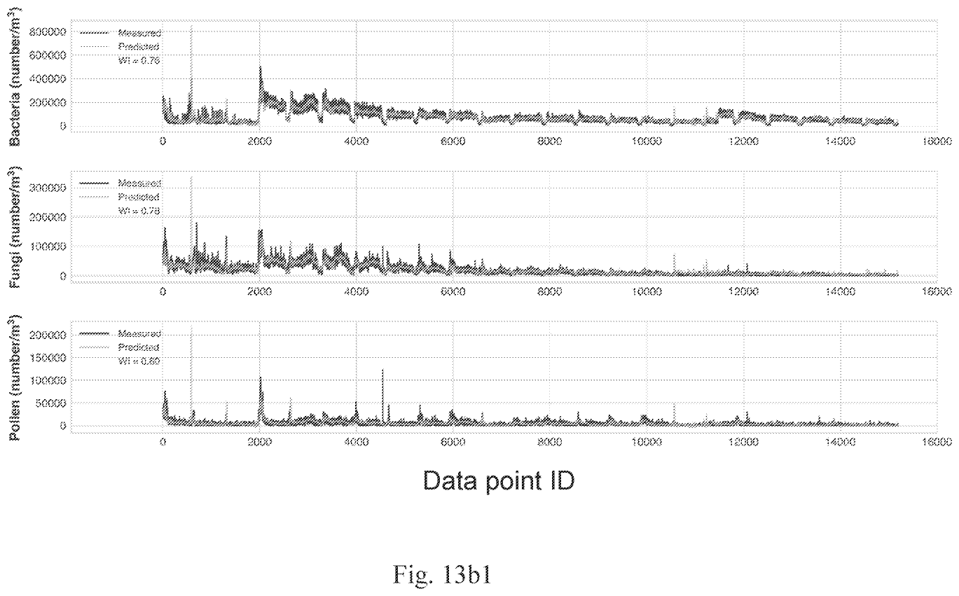

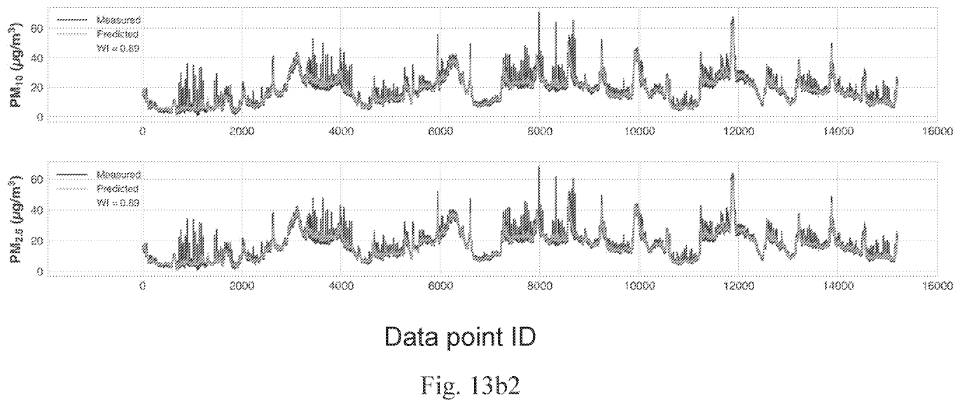

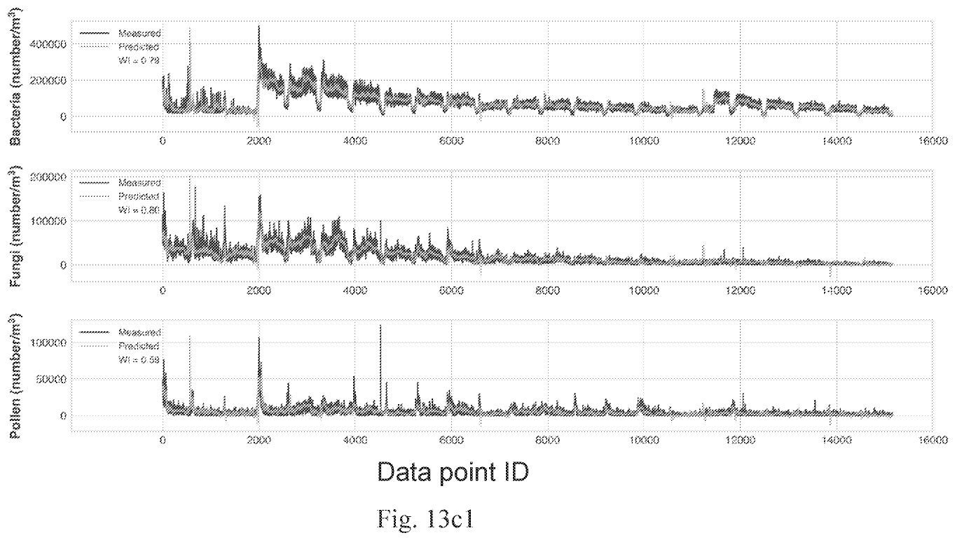

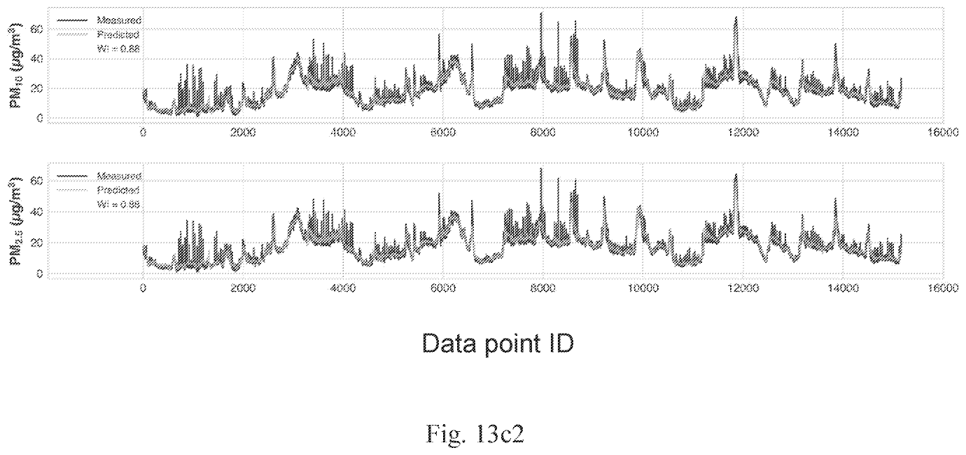

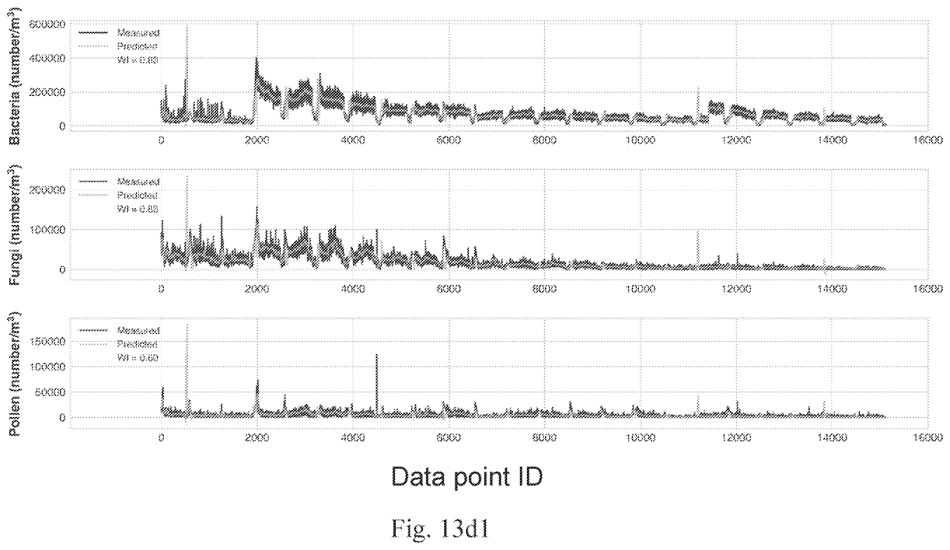

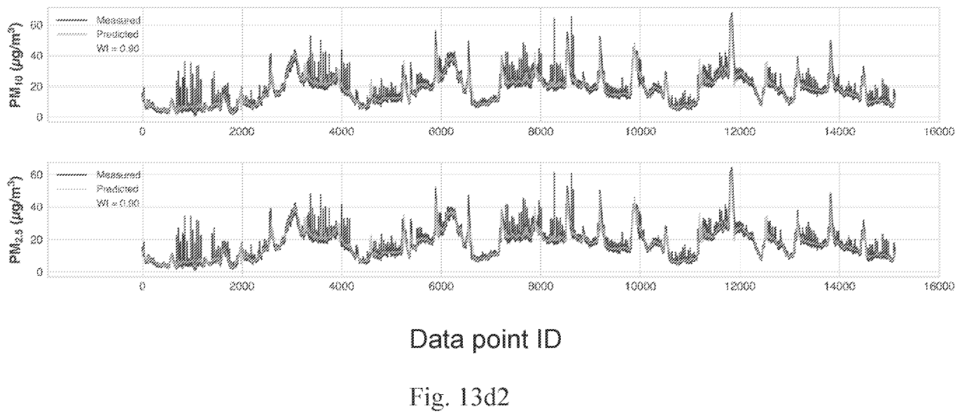

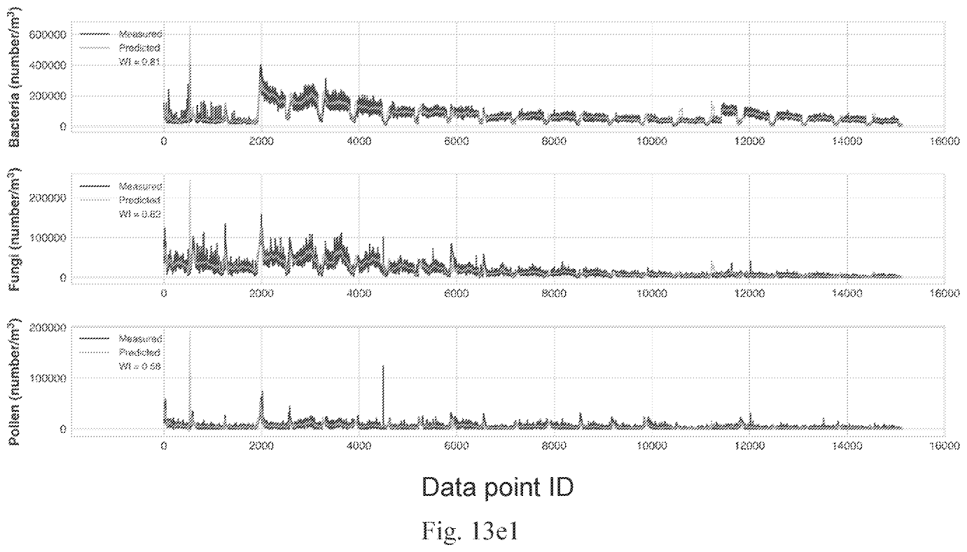

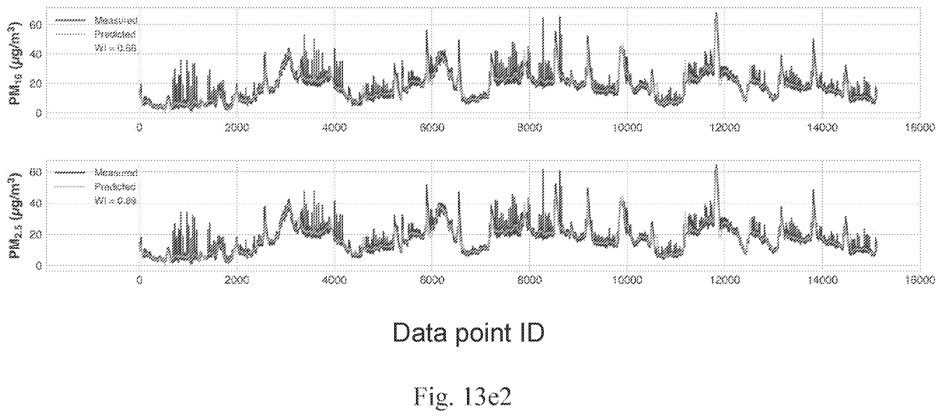

In one example, the ability of the trained LSTM model to make long-term continuous predictions for the commercial office (see FIGS. 12a1-12e2) and shopping mall (see FIGS. 13a1-13e2) for the five target features and for different time windows of input and output data was evaluated using the entire time series dataset of each venue. This revealed that the LSTM model captured the broad temporal trends in the five target features in all of the time windows and its average predictive accuracies of the time series and testing datasets were similar.

One can see that in the above embodiment, AI models are developed using physical and chemical data from an indoor air quality sensor and physical data from an ultraviolet light/laser-induced fluorescence instrument that measures bioaerosols. This enables effective determination of the concentrations of bioaerosols (bacteria, fungi and pollen) and 2.5-μm and 10-μm particulate matter (PM_{2.5} and PM₁₀) on a real-time and near-future (≤60 min) basis. Seven AI models are developed and evaluated using measured data from an operating commercial office and a shopping mall. The long short-term memory model required a relatively short training time and gave the highest prediction accuracy of ˜60%-80% for bioaerosols and ˜90% for PM on the testing and time series datasets from the two venues. The AI-based method for monitoring and predicting indoor concentrations of bioaerosols provides important information to building operators in an effective and economical manner, enabling them to optimally manage indoor environmental quality.

As a summary to the above embodiment, a LSTM model is developed and demonstrated that it could determine real-time and near-future concentrations of indoor bioaerosols and PM with an accuracy of ˜60%-80% and ˜90%, respectively, for both testing and time series datasets. It was expected that the predictive accuracy of PM would be relatively high as past PM data were used in the model. The ability of the model to continuously determine the real-time or near-future concentration of indoor bioaerosols relied on the deployment of an ultraviolet light/laser-induced fluorescence instrument to acquire the high temporal resolution biological data required for model training, as these data cannot be obtained by traditional culture-based methods. In comparison with the predictive accuracies that have been reported for concentrations of PM (˜80%-90%)^{37,38} and bacteria and fungi (based on culturing measurements; ˜50%-80%),^{22,39} the predictive accuracies for concentrations of these analytes generated by our LSTM model are similar or higher. As such, the embodiment above has demonstrated that AI models can use real-time IAQ sensor data to determine real-time and near-future concentrations of indoor bioaerosols and PM with relatively high accuracy. Given that preventing disease transmission in indoor environments is a top priority, there is an urgent need for low-cost technology that can accurately monitor IAQ, especially the concentrations of airborne bioaerosols.⁵² It has been shown that this could be achieved with a commercial IAQ sensor and an AI model, and could form a bioaerosol monitoring and forecasting system in large indoor environments. Such forecasting acts as an early-warning system for the high level of bioaerosols concentration, and would enable remedial actions (e.g., increasing fresh air supply) to be taken to maintain IAQ.

The exemplary embodiments are thus fully described. Although the description referred to particular embodiments, it will be clear to one skilled in the art that the invention may be practiced with variation of these specific details. Hence this invention should not be construed as limited to the embodiments set forth herein.

While the embodiments have been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

The functional units and modules of the systems and methods in accordance with the embodiments disclosed herein may be implemented using computing devices, computer processors, or electronic circuitries including but not limited to application-specific integrated circuits (ASIC), field programmable gate arrays (FPGA), and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance with the embodiments may be executed in one or more computing devices including server computers, personal computers, laptop computers, and mobile computing devices such as smartphones and tablet computers.

The embodiments include computer storage media, transient and non-transient memory devices having computer instructions or software codes stored therein which can be used to program computers or microprocessors to perform any of the processes of the present invention. The storage media, transient and non-transitory computer-readable storage medium can include but are not limited to floppy disks, optical discs, Blu-ray Disc, DVD, CD-ROMs, magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in a distributed fashion by one or more processing devices interconnected by a communication network, such as an intranet, WAN, LAN, the Internet, and other forms of data transmission medium.

In the preferred embodiments mentioned above, seven models were developed initially and the LSTM model was determined to be the best model for both commercial offices and shopping malls. However, the number of models or the LSTM model is by no means intended to be limiting. In variations of the embodiments, different number of models, and/or different types of models other than the seven models exemplified above can be used, and the best model can be different from the LSTM model in particular if the venue type is not a commercial office or a shopping center. The invention is not confined by any particular candidate models, or best models, but it is the approach that provides multiple candidate models and choosing a best one for each type of venue based on prediction accuracy that marks one of the essential features of the invention.

Also, in the preferred embodiments mentioned above, five different combinations of time windows of past measured data and predicted data are discussed in both evaluation of different AI models and actual monitoring/forecasting of indoor bioaerosol concentrations. However, those skilled in the art will realize that the number of time windows that can be used in the monitoring/forecasting operations is not limited to five, and the time length in each window may have a different value than what has been described above.

The invention may be applied to the monitoring/forecasting of bioaerosol concentrations in all types of indoor environment, and is not limited to the commercial office and the shopping center as described in the preferred embodiments above.

What is claimed is:

1. A method for predicting concentration of indoor bioaerosols, comprising steps of:
a) providing a plurality of artificial intelligence (AI) models;
b) evaluating a prediction accuracy of each of the plurality of AI models for a venue;
c) choosing a best model from the plurality of AI models for the venue;
d) inputting measured data at the venue into the best model; and
e) generating a prediction of concentration of indoor bioaerosols by the best model for the venue.

2. The method according to claim 1, wherein the plurality of AI models includes one or more of a linear regression model, a lasso regression model, a random forest (RF) model, an extreme gradient boosting model, a multilayer perceptron model, an LSTM model, and a recurrent neural network model.

3. The method according to claim 1, wherein Step B) further comprises steps of:
f) inputting test data for the venue into each of the plurality of AI models;
g) applying more than one pair of input and output time windows;
h) finding, for each of the plurality of AI model, a difference data between predicted test data and measured test data; and
i) determining one of the plurality of AI models that has a best difference data as the best model.

4. The method according to claim 3, wherein the difference data comprises one or more of a mean squared error (MSE), a root-mean-square error (RMSE) and a value on a revised version of the Willmott's index (WI).

5. The method according to claim 3, wherein the more than one pair of input and output time windows comprises a real-time window pair.

6. The method according to claim 1, wherein the measured data comprises a plurality of input features; the method further comprising a step of determining which one of the plurality of input features is more important than another one by conducting a permutation importance analysis.

7. The method according to claim 6, wherein the plurality of input features comprises one or more of temperature, relative humidity (RH), concentrations of CO₂, total volatile organic compounds (TVOCs), PM_{2.5} and PM₁₀.

8. The method according to claim 6, wherein the plurality of input features comprises concentrations of more than one biological matters.

9. An apparatus for predicting concentration of indoor bioaerosols, comprising:
a) one or more processors; and
b) a memory storing computer-executable instructions that, when executed, cause the one or more processors to
i) provide a plurality of artificial intelligence (AI) models;
ii) evaluate a prediction accuracy of each of the plurality of AI models for a venue;
iii) choose a best model from the plurality of AI models for the venue;
iv) input measured data at the venue into the best model; and
v) generate a prediction of concentration of indoor bioaerosols by the best model for the venue.

10. A non-transitory computer readable medium, comprising executable instructions that, when executed by at least one processor, direct the at least one processor to perform a method, the method comprising:
a) providing a plurality of artificial intelligence (AI) models;
b) evaluating a prediction accuracy of each of the plurality of AI models for a venue;
c) choosing a best model from the plurality of AI models for the venue;
d) inputting measured data at the venue into the best model; and
e) generating a prediction of concentration of indoor bioaerosols by the best model for the venue.
